(19) United States
(12) Patent Application Publication
(10) Pub. No.: US 2022/0389008 A1
(43) Pub. Date: Dec. 8, 2022

(54) 4-QUINOLINONE ANTIBACTERIAL COMPOUNDS

(71) Applicant: Janssen Sciences Ireland Unlimited Company, Co. Cork (IE)

(72) Inventors: Jérôme Émile Georges GUILLEMONT, Andé (FR); Magali Madeleine Simone MOTTE, Louviers (FR); Steffen Friedrich Walter WEIDNER, Illkirch-Graffenstaden (FR); Ellen Anita LANCKACKER, Lochristi (BE); Godelieve Maria J. LAMMENS, Geel (BE); Dirk Antonie LAMPRECHT, Turnhout (BE)

(21) Appl. No.: 17/764,727

(22) PCT Filed: Sep. 29, 2020
(86) PCT No.: PCT/EP2020/077173

§ 371 Date: Mar. 29, 2022

(30) Foreign Application Priority Data
Sep. 30, 2019 (EP) 19200366.3

(51) Int. Cl. C07D 471/04; A61P 31/06; A61K 31/4709; A61K 31/5377

(52) U.S. Cl. CPC C07D 471/04; A61P 31/06; A61K 31/4709; A61K 31/5377

(57) ABSTRACT

The present invention relates to the following compounds pounds (I) wherein the integers are as defined in the description, and where the compounds may be useful as medicaments, for instance for use in the treatment of tuberculosis (e.g. in combination).

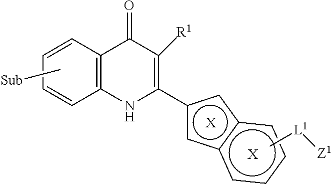

The present invention relates to novel compounds. The invention also relates to such compounds for use as a pharmaceutical and further for the use in the treatment of bacterial diseases, including diseases caused by pathogenic mycobacteria such as Mycobacterium tuberculosis. Such compounds may work by targeting the respiratory chain, and thereby blocking all energy production of mycobacteria. There are several ways of targeting the electron transport chain of mycobacteria, for instance by interfering with ATP synthase in M. tuberculosis. This particular invention focuses on the cytochrome bd target of the respiratory chain, which may be the primary mode of action. Hence, primarily, such compounds are antitubercular agents, and in particular may act as such when combined with another tuberculosis drug (e.g. another inhibitor of a different target of the electron transport chain).

### BACKGROUND OF THE INVENTION

Mycobacterium tuberculosis is the causative agent of tuberculosis (TB), a serious and potentially fatal infection with a world-wide distribution. Estimates from the World Health Organization indicate that more than 8 million people contract TB each year, and 2 million people die from tuberculosis yearly. In the last decade, TB cases have grown 20% worldwide with the highest burden in the most impoverished communities. If these trends continue, TB incidence will increase by 41% in the next twenty years. Fifty years since the introduction of an effective chemotherapy, TB remains after AIDS, the leading infectious cause of adult mortality in the world. Complicating the TB epidemic is the rising tide of multi-drug-resistant strains, and the deadly symbiosis with HIV. People who are HIV-positive and infected with TB are 30 times more likely to develop active TB than people who are HIV-negative and TB is responsible for the death of one out of every three people with HIV/AIDS worldwide.

Existing approaches to treatment of tuberculosis all involve the combination of multiple agents. For example, the regimen recommended by the U.S. Public Health Service is a combination of isoniazid, rifampicin and pyrazinamide for two months, followed by isoniazid and rifampicin alone for a further four months. These drugs are continued for a further seven months in patients infected with HIV. For patients infected with multi-drug resistant strains of M. tuberculosis, agents such as ethambutol, streptomycin, kanamycin, amikacin, capreomycin, ethionamide, cycloserine, ciprofoxacin and ofloxacin are added to the combination therapies. There exists no single agent that is effective in the clinical treatment of tuberculosis, nor any combination of agents that offers the possibility of therapy of less than six months' duration.

There is a high medical need for new drugs that improve current treatment by enabling regimens that facilitate patient and provider compliance. Shorter regimens and those that require less supervision are the best way to achieve this. Most of the benefit from treatment comes in the first 2 months, during the intensive, or bactericidal, phase when four drugs are given together; the bacterial burden is greatly reduced, and patients become noninfectious. The 4- to 6-month continuation, or sterilizing, phase is required to eliminate persisting bacilli and to minimize the risk of relapse. A potent sterilizing drug that shortens treatment to 2 months or less would be extremely beneficial. Drugs that facilitate compliance by requiring less intensive supervision also are needed. Obviously, a compound that reduces both the total length of treatment and the frequency of drug administration would provide the greatest benefit.

Complicating the TB epidemic is the increasing incidence of multi-drug-resistant strains or MDR-TB. Up to four percent of all cases worldwide are considered MDR-TB—those resistant to the most effective drugs of the four-drug standard, isoniazid and rifampin. MDR-TB is lethal when untreated and cannot be adequately treated through the standard therapy, so treatment requires up to 2 years of “second-line” drugs. These drugs are often toxic, expensive and marginally effective. In the absence of an effective therapy, infectious MDR-TB patients continue to spread the disease, producing new infections with MDR-TB strains. There is a high medical need for a new drug with a new mechanism of action, which is likely to demonstrate activity against drug resistant, in particular MDR strains.

The term “drug resistant” as used hereinbefore or hereinafter is a term well understood by the person skilled in microbiology. A drug resistant Mycobacterium is a Mycobacterium which is no longer susceptible to at least one previously effective drug; which has developed the ability to withstand antibiotic attack by at least one previously effective drug. A drug resistant strain may relay that ability to withstand to its progeny. Said resistance may be due to random genetic mutations in the bacterial cell that alters its sensitivity to a single drug or to different drugs.

MDR tuberculosis is a specific form of drug resistant tuberculosis due to a bacterium resistant to at least isoniazid and rifampicin (with or without resistance to other drugs), which are at present the two most powerful anti-TB drugs. Thus, whenever used hereinbefore or hereinafter “drug resistant” includes multi drug resistant.

Another factor in the control of the TB epidemic is the problem of latent TB. In spite of decades of tuberculosis (TB) control programs, about 2 billion people are infected by M. tuberculosis, though asymptomatically. About 10% of these individuals are at risk of developing active TB during their lifespan. The global epidemic of TB is fuelled by infection of HIV patients with TB and rise of multi-drug resistant TB strains (MDR-TB). The reactivation of latent TB is a high risk factor for disease development and accounts for 32% deaths in HIV infected individuals. To control TB epidemic, the need is to discover new drugs that can kill dormant or latent bacilli. The dormant TB can get reactivated to cause disease by several factors like suppression of host immunity by use of immunosuppressive agents like antibodies against tumor necrosis factor α or interferon-γ. In case of HIV positive patients the only prophylactic treatment available for latent TB is two-three months regimens of rifampicin, pyrazinamide. The efficacy of the treatment regime is still not clear and furthermore the length of the treatments is an important constrain in resource-limited environments. Hence there is a drastic need to identify new drugs, which can act as chemoprophylatic agents for individuals harboring latent TB bacilli.

The tubercle bacilli enter healthy individuals by inhalation; they are phagocytosed by the alveolar macrophages of the lungs. This leads to potent immune response and formation of granulomas, which consist of macrophages infected with M. tuberculosis surrounded by T cells. After a period of 6-8 weeks the host immune response cause death of infected cells by necrosis and accumulation of caseous material with certain extracellular bacilli, surrounded by macrophages, epitheloid cells and layers of lymphoid tissue at the periphery. In case of healthy individuals, most of the mycobacteria are killed in these environments but a small proportion of bacilli still survive and are thought to exist in a non-replicating, hypometabolic state and are tolerant to killing by anti-TB drugs like isoniazid. These bacilli can remain in the altered physiological environments even for individual's lifetime without showing any clinical symptoms of disease. However, in 10% of the cases these latent bacilli may reactivate to cause disease. One of the hypothesis about development of these persistent bacteria is patho-physiological environment in human lesions namely, reduced oxygen tension, nutrient limitation, and acidic pH. These factors have been postulated to render these bacteria phenotypically tolerant to major anti-mycobacterial drugs.

In addition to the management of the TB epidemic, there is the emerging problem of resistance to first-line antibiotic agents. Some important examples include penicillin-resistant Streptococcus pneumoniae, vancomycin-resistant enterococci, methicillin-resistant Staphylococcus aureus, multi-resistant salmonellae.

The consequences of resistance to antibiotic agents are severe. Infections caused by resistant microbes fail to respond to treatment, resulting in prolonged illness and greater risk of death. Treatment failures also lead to longer periods of infectivity, which increase the numbers of infected people moving in the community and thus exposing the general population to the risk of contracting a resistant strain infection. Hospitals are a critical component of the antimicrobial resistance problem worldwide. The combination of highly susceptible patients, intensive and prolonged antimicrobial use, and cross-infection has resulted in infections with highly resistant bacterial pathogens.

Self-medication with antimicrobials is another major factor contributing to resistance. Self-medicated antimicrobials may be unnecessary, are often inadequately dosed, or may not contain adequate amounts of active drug.

Patient compliance with recommended treatment is another major problem. Patients forget to take medication, interrupt their treatment when they begin to feel better, or may be unable to afford a full course, thereby creating an ideal environment for microbes to adapt rather than be killed.

Because of the emerging resistance to multiple antibiotics, physicians are confronted with infections for which there is no effective therapy. The morbidity, mortality, and financial costs of such infections impose an increasing burden for health care systems worldwide.

Therefore, there is a high need for new compounds to treat bacterial infections, especially mycobacterial infections including drug resistant and latent mycobacterial infections, and also other bacterial infections especially those caused by resistant bacterial strains.

There are several ways of targeting the electron transport chain of mycobacteria, for instance by interfering with ATP synthase in M. tuberculosis. Unlike many bacteria, M. tuberculosis is dependent on respiration to synthesise adequate amounts of ATP. Hence targeting the electron transport chain of the mycobacteria and thereby blocking energy production of mycobacteria is thought to be a potentially effective way of providing an efficient regimen against mycobacteria. Targets already known are ATP synthase inhibitors, as example of which is bedaquiline (marketed as Sirturo®), cytochrome bc inhibitors, examples of which include the compound Q203 described in Journal article Nature Medicine, 19, 1157-1160 (2013) by Pethe et al “Discovery of Q203, a potent clinical candidate for the treatment of tuberculosis”, as well as patent applications such as international patent applications WO 2017/001660, WO 2017/001661, WO 2017/216281 and WO 2017/216283.

Additionally, journal article Antimicrob.Agents Chemother, 2014, 6962-6965 by Arora et al describes compounds that target the respiratory bc₁ complex in M. tuberculosis, and where deletion of the cytochrome bd oxidase generated a hypersusceptible mutant. Journal article PANS (Early Edition), 2017, “Exploiting the synthetic lethality between terminal respiratory oxidases to kill Mycobacterium tuberculosis and clear host infection” by Kalia et al discloses various data around various tuberculosis compounds that target the respiratory chain. For instance, it is shown that the compound Q203 (a known bc inhibitor; see above) could inhibit mycobacteria completely and become bactericidal, after genetic deletion of the cytochrome bd oxidase-encoding genes CydAB. Similarly, journal article MBio, 2014 Jul. 15; 5(4) by Berney et al “A Mycobacterium tuberculosis cytochrome bd oxidase mutant is hypersensitive to bedaquiline” shows that the activity of bedaquiline is enhanced when bd is inactivated.

One known cytochrome bd inhibitor is Aurachin D, which is a quinolone with a relatively long side-chain. Cytochrome bd itself is not essential for aerobic growth, but is upregulated and protects against a variety of stresses in various bacterial strains, for example as described in journal article Biochimica et Biophysica Acta 1837 (2014) 1178-1187 by Giuffrè et al. Hence, monotherapy with a cytochrome bd inhibitor would not necessarily be expected to inhibit mycobacteria growth, but a combination with another inhibitor of a target of the electron transport chain of mycobacteria could be.

Various compounds are described in international patent applications WO 2012/069856 and WO 2017/103615, with the latter application describing such compounds as cytochrome bd inhibitors and indicates that therapeutic combination products comprising one or more respiratory electron transport chain inhibitor and a cytochrome bd inhibitor is disclosed. Specifically, the compound CK-2-63 is described as a cytochrome bd inhibitor showing various inhibitor activity data, and combination data is also disclosed including combination of CK-2-63 with a mycobacterium cytochrome bcc inhibitor (e.g. AWE-402, where it is indicated therein that it is structurally related to the cytochrome bcc inhibitor Q203). It is indicated that such dual combination led to in increase in mycobacteria kill. It also described a combination of bedaquiline (a known ATP synthase inhibitor) with CK-2-63, and it is indicated that CK-2-63 showed an enhancement of bedaquiline activity at low concentrations. Data around a triple combination of bedaquiline, AWE-402 (a bc inhibitor; see above) and CK-2-63 is also shown.

This particular invention focuses on novel compounds of the cytochrome bd target of the respiratory chain. New alternative/improved compounds are required, which may be tested/employed for use in combination.

### SUMMARY OF THE INVENTION

There is now provided a compound of formula (I)

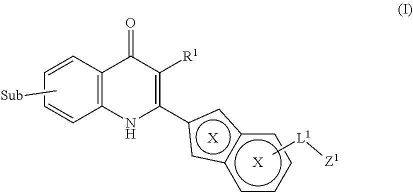

wherein

R¹ represents C₁₋₆ alkyl, —Br, hydrogen or —C(O)N(R^{q1})R^{q2};

R^{q1} and R^{q2} independently represent hydrogen or C₁₋₆ alkyl, or may be linked together to form a 3-6 membered carbocyclic ring optionally substituted by one or more C₁₋₃ alkyl substituents;

Sub represents one or more optional substituents selected from halo, —CN, C₁₋₆ alkyl and —O—C₁₋₆ alkyl (wherein the latter two alkyl moieties are optionally substituted by one or more fluoro atoms);

the two “X” rings together represent a 9-membered bicyclic heteroaryl ring (consisting of a 5-membered aromatic ring fused to another 6-membered aromatic ring), which bicyclic heteroaryl ring contains between one and four heteroatoms (e.g. selected from nitrogen, oxygen and sulfur), and which bicyclic ring is optionally substituted by one or more substituents selected from halo and C₁₋₆ alkyl (itself optionally substituted by one or more fluoro atoms);

L¹ represents an optional linker group, and hence may be a direct bond, —O—, —OCH₂—, —C(R^{x1})(R^{x2})— or —C(O)—N(H)—CH₂—;

R^{x1} and R^{x2} independently represent hydrogen or C₁₋₃ alkyl;

Z¹ represents any one of the following moieties:

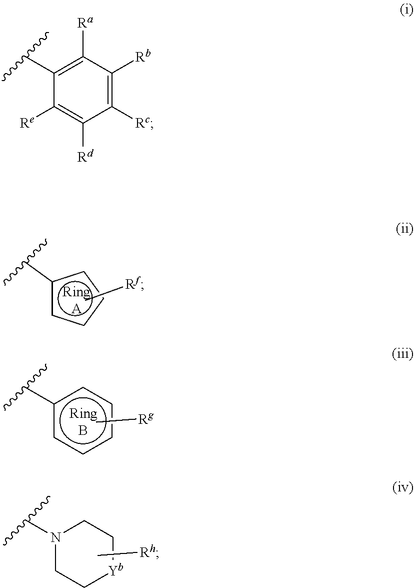

(v) perfluoro C₁₋₃ alkyl (e.g. —CF₃);
(vi) —F, —Br, or —CN;

ring A represents a 5-membered aromatic ring containing at least one heteroatom (preferably containing at least one nitrogen atom), and which ring is optionally substituted by one or more substituents independently selected from R^{f};

ring B represents a 6-membered aromatic ring containing at least one heteroatom (preferably containing at least one nitrogen atom), and which ring is optionally substituted by one or more substituents independently selected from R^{g};

Y^{b} represents —CH₂ or NH, and R^{h} represents one or more substituents on the 6-membered N and Y^{b}-containing ring (which R^{h} substituents may also be present on Y^{b});

R^{a}, R^{b}, R^{c}, R^{d} and R^{e} independently represent hydrogen or a substituent selected from B¹;

each R^{f}, each R^{g} and each R^{h} (which are optional substituents), when present, independently represent a substituent selected from B¹;

each B¹ independently represents a substituent selected from:
(i) halo;
(ii) —R^{d1};
(iii) —OR^{e1};
(iv) —C(O)N(R^{e2})R^{e3}
(v) —SF₅;
(vi) —N(R^{e4})S(O)₂R^{e5};

R^{d1} represents C₁₋₆ alkyl optionally substituted by one or more halo (e.g. fluoro) atoms;

R^{e1}, R^{e2}, R^{e3}, R^{e4} and R^{e5} each independently represent hydrogen or C₁₋₆ alkyl optionally substituted by one or more fluoro atoms;

or a pharmaceutically-acceptable salt thereof,

which compounds may be referred to herein as “compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable acid addition salts as mentioned hereinabove are meant to comprise the therapeutically active non-toxic acid addition salt forms that the compounds of formula (I) are able to form. These pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

For the purposes of this invention solvates, prodrugs, N-oxides and stereoisomers of compounds of the invention are also included within the scope of the invention.

The term “prodrug” of a relevant compound of the invention includes any compound that, following oral or parenteral administration, is metabolised in vivo to form that compound in an experimentally-detectable amount, and within a predetermined time (e.g. within a dosing interval of between 6 and 24 hours (i.e. once to four times daily)). For the avoidance of doubt, the term “parenteral” administration includes all forms of administration other than oral administration.

Prodrugs of compounds of the invention may be prepared by modifying functional groups present on the compound in such a way that the modifications are cleaved, in vivo when such prodrug is administered to a mammalian subject. The modifications typically are achieved by synthesising the parent compound with a prodrug substituent. Prodrugs include compounds of the invention wherein a hydroxyl, amino, sulfhydryl, carboxy or carbonyl group in a compound of the invention is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. 1-92, Elesevier, New York-Oxford (1985).

Compounds of the invention may contain double bonds and may thus exist as E (entgegen) and Z (zusammen) geometric isomers about each individual double bond. Positional isomers may also be embraced by the compounds of the invention. All such isomers (e.g. if a compound of the invention incorporates a double bond or a fused ring, the cis- and trans-forms, are embraced) and mixtures thereof are included within the scope of the invention (e.g. single positional isomers and mixtures of positional isomers may be included within the scope of the invention).

Compounds of the invention may also exhibit tautomerism. All tautomeric forms (or tautomers) and mixtures thereof are included within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerisations. Valence tautomers include interconversions by reorganisation of some of the bonding electrons.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person.

All stereoisomers (including but not limited to diastereoisomers, enantiomers and atropisomers) and mixtures thereof (e.g. racemic mixtures) are included within the scope of the invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

The compounds of the present invention may exist in unsolvated as well as solvated forms with pharmaceutically acceptable solvents such as water, ethanol, and the like, and it is intended that the invention embrace both solvated and unsolvated forms.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸O, ³²P, ³³P, ³⁵S, ¹⁸F, ³⁶Cl, ¹²³I, and ¹²⁵I. Certain isotopically-labeled compounds of the present invention (e.g., those labeled with ³H and ¹⁴C) are useful in compound and for substrate tissue distribution assays. Tritiated (³H) and carbon-14 (¹⁴C) isotopes are useful for their ease of preparation and detectability. Further, substitution with heavier isotopes such as deuterium (i.e., ²H may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the description/Examples hereinbelow, by substituting an isotopically labeled reagent for a non-isotopically labeled reagent.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q}-cycloalkyl group). Such cycloalkyl groups may be monocyclic or bicyclic and may further be bridged. Further, when there is a sufficient number (i.e. a minimum of four) of carbon atoms, such groups may also be part cyclic. Such alkyl groups may also be saturated or, when there is a sufficient number (i.e. a minimum of two) of carbon atoms, be unsaturated (forming, for example, a C_{2-q} alkenyl or a C_{2-q}alkynyl group).

C_{3-q} cycloalkyl groups (where q is the upper limit of the range) that may be specifically mentioned may be monocyclic or bicyclic alkyl groups, which cycloalkyl groups may further be bridged (so forming, for example, fused ring systems such as three fused cycloalkyl groups). Such cycloalkyl groups may be saturated or unsaturated containing one or more double bonds (forming for example a cycloalkenyl group). Substituents may be attached at any point on the cycloalkyl group. Further, where there is a sufficient number (i.e. a minimum of four) such cycloalkyl groups may also be part cyclic.

The term “halo”, when used herein, preferably includes fluoro, chloro, bromo and iodo.

Heterocyclic groups when referred to herein may include aromatic or non-aromatic heterocyclic groups, and hence encompass heterocycloalkyl and hetereoaryl. Equally, “aromatic or non-aromatic 5- or 6-membered rings” may be heterocyclic groups (as well as carbocyclic groups) that have 5- or 6-members in the ring.

Heterocycloalkyl groups that may be mentioned include non-aromatic monocyclic and bicyclic heterocycloalkyl groups in which at least one (e.g. one to four) of the atoms in the ring system is other than carbon (i.e. a heteroatom), and in which the total number of atoms in the ring system is between 3 and 20 (e.g. between three and ten, e.g between 3 and 8, such as 5- to 8-). Such heterocycloalkyl groups may also be bridged. Further, such heterocycloalkyl groups may be saturated or unsaturated containing one or more double and/or triple bonds, forming for example a C_{2-q} heterocycloalkenyl (where q is the upper limit of the range) group. C_{2-q} heterocycloalkyl groups that may be mentioned include 7-azabicyclo[2.2.1]heptanyl, 6-azabicyclo[3.1.1]heptanyl, 6-azabicyclo[3.2.1]-octanyl, 8-azabicyclo-[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo-[3.2.1]octanyl, oxetanyl, oxiranyl, piperazinyl, piperidinyl, non-aromatic pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like.

Substituents on heterocycloalkyl groups may, where appropriate, be located on any atom in the ring system including a heteroatom. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N— or S— oxidised form. Heterocycloalkyl mentioned herein may be stated to be specifically monocyclic or bicyclic.

Aromatic groups may be aryl or heteroaryl. Aryl groups that may be mentioned include C₆₋₂₀, such as C₆₋₁₂ (e.g. C₆₋₁₀) aryl groups. Such groups may be monocyclic, bicyclic or tricyclic and have between 6 and 12 (e.g. 6 and 10) ring carbon atoms, in which at least one ring is aromatic. C₆₋₁₀ aryl groups include phenyl, naphthyl and the like, such as 1,2,3,4-tetrahydronaphthyl. The point of attachment of aryl groups may be via any atom of the ring system. For example, when the aryl group is polycyclic the point of attachment may be via atom including an atom of a non-aromatic ring. However, when aryl groups are polycyclic (e.g. bicyclic or tricyclic), they are preferably linked to the rest of the molecule via an aromatic ring. Most preferred aryl groups that may be mentioned herein are “phenyl”.

Unless otherwise specified, the term “heteroaryl” when used herein refers to an aromatic group containing one or more heteroatom(s) (e.g. one to four heteroatoms) preferably selected from N, O and S. Heteroaryl groups include those which have between 5 and 20 members (e.g. between 5 and 10) and may be monocyclic, bicyclic or tricyclic, provided that at least one of the rings is aromatic (so forming, for example, a mono-, bi-, or tricyclic heteroaromatic group). When the heteroaryl group is polycyclic the point of attachment may be via any atom including an atom of a non-aromatic ring. However, when heteroaryl groups are polycyclic (e.g. bicyclic or tricyclic), they are preferably linked to the rest of the molecule via an aromatic ring. Heteroaryl groups that may be mentioned include 3,4-dihydro-1H-isoquinolinyl, 1,3-dihydroisoindolyl, 1,3-dihydroisoindolyl (e.g. 3,4-dihydro-1H-isoquinolin-2-yl, 1,3-dihydroisoindol-2-yl, 1,3-dihydroisoindol-2-yl; i.e. heteroaryl groups that are linked via a non-aromatic ring), or, preferably, acridinyl, benzimidazolyl, benzodioxanyl, benzodioxepinyl, benzo-dioxolyl (including 1,3-benzodioxolyl), benzofuranyl, benzofurazanyl, benzothiadiazolyl (including 2,1,3-benzothiadiazolyl), benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetra-hydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N— or S— oxidised form. Heteroaryl groups mentioned herein may be stated to be specifically monocyclic or bicyclic. When heteroaryl groups are polycyclic in which there is a non-aromatic ring present, then that non-aromatic ring may be substituted by one or more ═O group. Most preferred heteroaryl groups that may be mentioned herein are 5- or 6-membered aromatic groups containing 1, 2 or 3 heteroatoms (e.g. preferably selected from nitrogen, oxygen and sulfur).

It may be specifically stated that the heteroaryl group is monocyclic or bicyclic. In the case where it is specified that the heteroaryl is bicyclic, then it may consist of a five-, six- or seven-membered monocyclic ring (e.g. a monocyclic heteroaryl ring) fused with another five-, six- or seven-membered ring (e.g. a monocyclic aryl or heteroaryl ring).

Heteroatoms that may be mentioned include phosphorus, silicon, boron and, preferably, oxygen, nitrogen and sulfur.

When “aromatic” groups are referred to herein, they may be aryl or heteroaryl. When “aromatic linker groups” are referred to herein, they may be aryl or heteroaryl, as defined herein, are preferably monocyclic (but may be polycyclic) and attached to the remainder of the molecule via any possible atoms of that linker group. However, when, specifically carbocyclic aromatic linker groups are referred to, then such aromatic groups may not contain a heteroatom, i.e. they may be aryl (but not heteroaryl).

For the avoidance of doubt, where it is stated herein that a group may be substituted by one or more substituents (e.g. selected from C₁₋₆ alkyl), then those substituents (e.g. alkyl groups) are independent of one another. That is, such groups may be substituted with the same substituent (e.g. same alkyl substituent) or different (e.g. alkyl) substituents.

All individual features (e.g. preferred features) mentioned herein may be taken in isolation or in combination with any other feature (including preferred feature) mentioned herein (hence, preferred features may be taken in conjunction with other preferred features, or independently of them).

The skilled person will appreciate that compounds of the invention that are the subject of this invention include those that are stable. That is, compounds of the invention include those that are sufficiently robust to survive isolation from e.g. a reaction mixture to a useful degree of purity.

Preferred compounds of the invention include those in which:

when R¹ represents —C(O)N(R^{q1})R^{q2}, then R^{q1} and R^{q2} independently represent hydrogen or C₁₋₃ alkyl (so forming e.g. —C(O)N(H)CH₃ or —C(O)N(CH₃)₂);

R¹ in an embodiment, represents hydrogen, C₁₋₆ alkyl or —C(O)N(R^{q1})R^{q2};

one of R^{q1} and R^{q2} represents C₁₋₃ alkyl (e.g. methyl) and the other represents hydrogen or C₁₋₃ alkyl (e.g. methyl);

R¹, in a further embodiment, represents C₁₋₆ alkyl, e.g. C₁₋₃ alkyl such as methyl;

Sub is not present, i.e. there are no further substituents on the relevant aromatic/benzene ring, or represents one or two substituents selected from halo (e.g. fluoro and/or chloro) and —OC₁₋₃ alkyl (e.g. —OCH₃).

In an embodiment, R¹ represents C₁₋₃ alkyl, such as methyl.

In an embodiment, Sub is not present, i.e. the relevant aromatic/benzene ring does not contain any further substituents.

Compounds of the invention contain a 9-membered bicyclic heteroaromatic group represented by the “X” rings. In an embodiment, further compounds of the invention include those in which such bicyclic ring:

contains at least one nitrogen atom (in an embodiment, at the ring junction); and/or contains one, two, three or four heteroatoms in total (for instance, the 9-membered ring contains one, two or three nitrogen heteroatoms); and/or

in addition to being substituted by L¹, is optionally further substituted by one or two (e.g. one) further substituent selected from C₁₋₃ alkyl and —OC₁₋₃ alkyl (in which the latter two alkyl moieties are each optionally substituted with fluoro, so forming e.g. a —CF₃, —OCF₃ or —OCH₃ substituent).

In an embodiment of the invention, compounds of the invention are those in which the “X” rings (the bicyclic heteroaryl group) are represented by a sub-formula (IB) as defined hereinbelow (where it will be appreciated that the rules of valency will be adhered to, e.g. where C is mentioned then it may need to have a H attached to it), in which:

one of X¹ and X² represents N (i.e. there is an essential nitrogen at the ring junction) and the other represents C;

the other integers X³, X⁴ and X⁵ may represent C (or CH) or a heteroatom (such as N, O and/or S; and, in an embodiment, N); and/or

none, any one or two of X³, X⁴ and X⁵ represents a heteroatom (e.g. N, O and/or S; and, in an embodiment, N) and the other(s) represents C (or CH).

Hence, in view of the foregoing, preferred compounds of the invention include those in which:

one of X¹ and X² represents N; and

none, one or two of X³, X⁴ and X⁵ represents N.

The “X” rings in compounds of the invention (the 9-membered bicyclic heteroaryl group) may be depicted as follows (in which the left hand side would be further bound to the requisite quinolinone or formula (I) and the right hand side would be further bound to the L¹ group of formula (I):

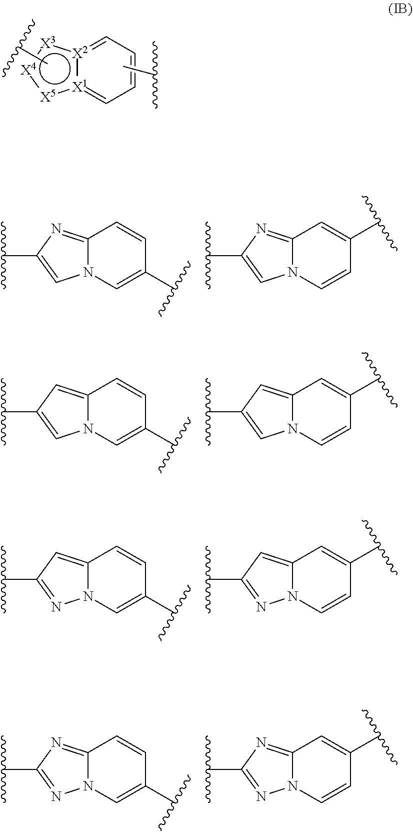

In a further embodiment, preferred compounds of the invention include those in which in the sub-formula (TB) depicted above:

any three of X¹, X², X³, X⁴ and X⁵ represent a heteroatom (e.g. nitrogen) and the other two represent C (or CH);

one of X¹ and X² represents N (i.e. there is an essential nitrogen at the ring junction) and the other represents C;

none, any one or any two of X³, X⁴ and X⁵ represents a N heteroatom and the other(s) represents C (or CH); and/or

the 9-membered bicyclic heteroaryl group depicted by the “X” rings are as defined in the formulae above,

and in which in all of the cases above, it will be understood that the rules of valency will need to be adhered to.

In a further embodiment, preferred compounds of the invention include those in which in the sub-formula (TB) depicted above:

X¹, X³ and X⁵ represent a heteroatom (e.g. nitrogen) and X² and X⁴ represent C (or CH).

In a preferred embodiment, the “X” rings in compounds of the invention (the 9-membered bicyclic heteroaryl group) may be depicted as follows (in which the left hand side would be further bound to the requisite quinolinone or formula (I) and the right hand side would be further bound to the L¹ group of formula (I):

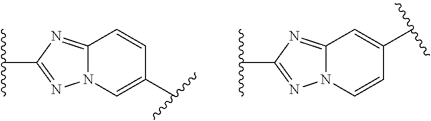 [SMILES: *c1ccc2nc(*)nn2c1 ; *c1ccn2nc(*)nc2c1]

Other preferred compounds of the invention include those in which:

L¹ represents a direct bond, —O—, —OCH₂— —C(R^{x1})(R^{x2})— or —C(O)—N(H)—CH₂—;

R^{x1} and R^{x2} independently represent hydrogen; for example:

L¹ may specifically represent a direct bond, —O—, —OCH₂— or —CH₂— (or, in a more specific embodiment, a direct bond, —O— or —CH₂—; especially a direct bond or —CH₂—).

In an embodiment, L¹ represents a direct bond.

In embodiments of the invention, Z¹ represents:

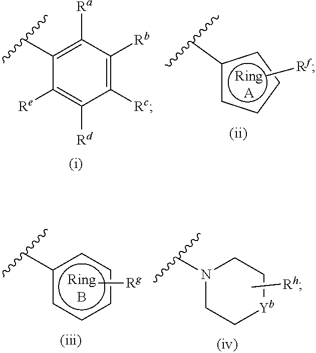

(v) perfluoro C₁₋₃ alkyl (e.g. —CF₃); or
(vi) —F, —Br, —Cl or —CN;

and hence there are six embodiments of the invention, and in an aspect, Z¹ represents (i), (ii) or (iii) (e.g. Z¹ represents (i) or (ii)) and, in a further aspect, Z¹ represents (iv) and, in a separate embodiment, Z¹ represents (v) or (vi) (e.g. Z¹ represents (v)). Hence, in an embodiment, Z¹ represents an aromatic ring (i.e. (i), (ii) or (iii) above), for instance (i) or (ii).

In an embodiment, Z¹ represents (i), i.e. phenyl bearing R^{a} to R^{e}.

In a further embodiment, compounds of the invention include those in which:

when ring A is present, it represents a 5-membered aromatic ring, it contains one, two or three heteroatoms preferably selected from nitrogen, oxygen and sulfur; in a further embodiment, such ring is optionally substituted by one or two substituents independently selected from R^{f};

when ring B is present, it represents a 6-membered aromatic ring containing one nitrogen atom; and, in a further embodiment, such ring is optionally substituted by one or two substituents independently selected from R^{g};

Y^{b} represents —CH₂ or NH, and R^{h} represents one or two substituents on the 6-membered N and Y^{b}-containing ring (which R^{h} substituents may also be present on Y^{b});

R^{a}, R^{b}, R^{c}, R^{d} and R^{e} independently represent hydrogen or a substituent selected from B¹;

R^{f}, R^{g} and R^{h} each independently represent a substituent selected from B¹.

In an embodiment, when Ring A is present (i.e. Z¹ represents (ii)), then such aromatic 5-membered (optionally substituted) ring may: (i) contain one sulfur atom (so forming a thienyl); (ii) contain one nitrogen and one sulfur atom (so forming e.g. thiazolyl); (iii) contain two nitrogen atoms (so forming e.g. a pyrazolyl); (iv) contains two nitrogen atoms and one sulfur atom; (v) contains two nitrogen atoms and one oxygen atom; (vi) contains three nitrogen atoms. It may also contain one oxygen atom (so forming, e.g. oxazolyl).

In an embodiment, when Ring B is present (i.e. Z¹ represents (iii)), then such aromatic 6-membered ring may contain one nitrogen atom, so forming a pyridyl group (e.g. a 3-pyridyl group).

In an embodiment, further preferred compounds of the inventions include those in which:

none, but preferably, one or two (e.g. one) of R^{a}, R^{b}, R^{c}, R^{d} and R^{e} represents B¹ and the others represent hydrogen; and/or

one of R^{b}, R^{c} and R^{d} (preferably R^{c}) represents B¹ and the others represent hydrogen.

In a further embodiment, compounds of the inventions include those in which R^{b} and one of R^{c} or R^{d} independently represent B¹; and R^{a}, R^{e} and the other R^{c} or R^{d} (that does not represent B¹) represent hydrogen.

In a further embodiment, yet further preferred compounds of the inventions include those in which:

B¹ represents a substituent selected from:
(i) fluoro;
(ii) —OR^{e1};
(iii) C₁₋₃ alkyl, optionally substituted by one or more fluoro atom;
(iv) —C(O)N(R^{e2})R^{e3};
(v) —N(R^{e4})S(O)₂R^{e5};
(vi) —SF₅;

R^{e2} and R^{e4} independently represent hydrogen;

R^{e1}, R^{e3} and R^{e5} each independently represent C₁₋₃ alkyl (e.g. methyl) (e.g. optionally) substituted by one or more fluoro atoms.

In a further embodiment of the invention, B¹ represents a substituent selected from halo (e.g. fluoro), C₁₋₃ alkyl (optionally substituted by one or more fluoro atom) and —OR^{e1} (in which R^{e1} represents C₁₋₃ alkyl optionally substituted by one or more fluoro atom, so forming e.g. —OCF₃). In a specific embodiment, B¹ is selected from fluoro, —CH₃, —OCH₃, —CF₃, —CHF₂, —CH₂CF₃, —CH₂CHF₂, and —OCF₃. In a further specific embodiment, B¹ is selected from fluoro, —CH₃, —CF₃, —CH₂CF₃ and —OCF₃.

In a particular embodiment of the invention, compounds contain one B¹ group preferably selected from fluoro, —CH₂CF₃, —OCH₃ and —OCF₃ (preferably further selected from fluoro and —OCF₃).

In a particular embodiment of the invention, compounds contain two B¹ group (preferably selected from fluoro, —CH₃, —CF₃, and —OCH₃).

### Pharmacology

The compounds according to the invention have surprisingly been shown to be suitable for the treatment of a bacterial infection including a mycobacterial infection, particularly those diseases caused by pathogenic mycobacteria such as Mycobacterium tuberculosis (including the latent and drug resistant form thereof). The present invention thus also relates to compounds of the invention as defined hereinabove, for use as a medicine, in particular for use as a medicine for the treatment of a bacterial infection including a mycobacterial infection.

Such compounds of the invention may act by interfering with ATP synthase in M. tuberculosis, with the inhibition of cytochrome bd activity being the primary mode of action. Such bd inhibition may have an effect in killing mycobacteria (and hence having an anti-tuberculosis effect directly). However, as cytochrome bd is not necessarily essential for aerobic growth, it may have the most pronounced effect in combination with another inhibitor of a target of the electron transport chain of mycobacteria. Such compounds may be tested for cytochrome bd activity by testing in an enzymatic assay, and may also be tested for activity in the treatment of a bacterial infection (e.g. mycobacterial infection) by testing the kill kinetics, for example of such compounds alone or in combination (as mentioned herein, e.g. with one or more other inhibitor(s) of a (different) target of the electron transport chain of mycobacteria; such other different targets may be more implicated in aerobic growth).

Cytochrome bd is a component of the electron transport chain, and therefore may be implicated with ATP synthesis, for instance alone or especially with one or more other inhibitor(s) of a target of the electron transport chain of mycobacteria.

Further, the present invention also relates to the use of a compound of the invention, as well as any of the pharmaceutical compositions thereof as described hereinafter for the manufacture of a medicament for the treatment of a bacterial infection including a mycobacterial infection (for instance when such compound of the invention is used in combination with another inhibitor of a target of the electron transport chain of mycobacteria).

Accordingly, in another aspect, the invention provides a method of treating a patient suffering from, or at risk of, a bacterial infection, including a mycobacterial infection, which comprises administering to the patient a therapeutically effective amount of a compound or pharmaceutical composition according to the invention (for instance a therapeutically effective amount of a compound or pharmaceutical composition of the invention, in combination with one or more other inhibitor(s) of a target of the electron transport chain of mycobacteria).

The compounds of the present invention also show activity against resistant bacterial strains (for instance alone or in combination with another inhibitor of a target of the electron transport chain of mycobacteria).

Whenever used hereinbefore or hereinafter, that the compounds can treat a bacterial infection (alone or in combination) it is meant that the compounds can treat an infection with one or more bacterial strains.

The invention also relates to a composition comprising a pharmaceutically acceptable carrier and, as active ingredient, a therapeutically effective amount of a compound according to the invention. The compounds according to the invention may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, in particular, for administration orally or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations which are intended to be converted, shortly before use, to liquid form preparations.

Depending on the mode of administration, the pharmaceutical composition will preferably comprise from 0.05 to 99% by weight, more preferably from 0.1 to 70% by weight, even more preferably from 0.1 to 50% by weight of the active ingredient(s), and, from 1 to 99.95% by weight, more preferably from 30 to 99.9% by weight, even more preferably from 50 to 99.9% by weight of a pharmaceutically acceptable carrier, all percentages being based on the total weight of the composition.

The pharmaceutical composition may additionally contain various other ingredients known in the art, for example, a lubricant, stabilising agent, buffering agent, emulsifying agent, viscosity-regulating agent, surfactant, preservative, flavouring or colorant.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof. The daily dosage of the compound according to the invention will, of course, vary with the compound employed, the mode of administration, the treatment desired and the mycobacterial disease indicated. However, in general, satisfactory results will be obtained when the compound according to the invention is administered at a daily dosage not exceeding 1 gram, e.g. in the range from 10 to 50 mg/kg body weight.

Given the fact that the compounds of the invention are useful against bacterial infections, the present compounds may be combined with other antibacterial agents in order to effectively combat bacterial infections. Where it is indicated that compounds may be useful against bacterial infections, we mean that those compounds may have activity as such or those compounds may be effective in combination (as described herein, e.g. with one or more other inhibitors of the electron transport chain of mycobacteria) by enhancing activity or providing synergistic combinations, for example as may be described in the experimental hereinafter.

Therefore, the present invention also relates to a combination of (a) a compound according to the invention, and (b) one or more other antibacterial agents (e.g. one or more other inhibitors of the electron transport chain of mycobacteria, for instance a cytochrome bc inhibitor, an ATP synthase inhibitor, a NDH2 inhibitor and/or an inhibitor of the menaquinone synthesis pathway, such as a MenG inhibitor). The present invention also relates to such a compound or combination, for use as a medicine.

The present invention also relates to the use of a combination or pharmaceutical composition as defined directly above for the treatment of a bacterial infection.

A pharmaceutical composition comprising a pharmaceutically acceptable carrier and, as active ingredient, a therapeutically effective amount of (a) a compound according to the invention, and (b) one or more other antibacterial agents (e.g. one or more other inhibitors of the electron transport chain of mycobacteria, for instance a cytochrome bc inhibitor, an ATP synthase inhibitor, a NDH2 inhibitor and/or an inhibitor of the menaquinone synthesis pathway, such as a MenG inhibitor), is also comprised by the present invention.

The weight ratio of (a) the compound according to the invention and (b) the other antibacterial agent(s) when given as a combination may be determined by the person skilled in the art. Said ratio and the exact dosage and frequency of administration depends on the particular compound according to the invention and the other antibacterial agent(s) used, the particular condition being treated, the severity of the condition being treated, the age, weight, gender, diet, time of administration and general physical condition of the particular patient, the mode of administration as well as other medication the individual may be taking, as is well known to those skilled in the art. Furthermore, it is evident that the effective daily amount may be lowered or increased depending on the response of the treated subject and/or depending on the evaluation of the physician prescribing the compounds of the instant invention. A particular weight ratio for the present compound of the invention and another antibacterial agent may range from 1/10 to 10/1, more in particular from 1/5 to 5/1, even more in particular from 1/3 to 3/1.

The compounds according to the invention and the one or more other antibacterial agents may be combined in a single preparation or they may be formulated in separate preparations so that they can be administered simultaneously, separately or sequentially. Thus, the present invention also relates to a product containing (a) a compound according to the invention, and (b) one or more other antibacterial agents (e.g. one or more other inhibitors of the electron transport chain of mycobacteria, for instance a cytochrome bc inhibitor, an ATP synthase inhibitor, a NDH2 inhibitor and/or an inhibitor of the menaquinone synthesis pathway, such as a MenG inhibitor), as a combined preparation for simultaneous, separate or sequential use in the treatment of a bacterial infection.

The other antibacterial agents which may be combined with the compounds of the invention are for example antibacterial agents known in the art. For example, the compounds of the invention may be combined with antibacterial agents known to interfere with the respiratory chain of Mycobacterium tuberculosis, including for example direct inhibitors of the ATP synthase (e.g. bedaquiline, bedaquiline fumarate or any other compounds that may have be disclosed in the prior art, e.g. compounds disclosed in WO2004/011436), inhibitors of ndh2 (e.g. clofazimine) and inhibitors of cytochrome bd. Additional mycobacterial agents which may be combined with the compounds of the invention are for example rifampicin (=rifampin); isoniazid; pyrazinamide; amikacin; ethionamide; ethambutol; streptomycin; para-aminosalicylic acid; cycloserine; capreomycin; kanamycin; thioacetazone; PA-824; delamanid; quinolones/fluoroquinolones such as for example moxifloxacin, gatifloxacin, ofloxacin, ciprofloxacin, sparfloxacin; macrolides such as for example clarithromycin, amoxycillin with clavulanic acid; rifamycins; rifabutin; rifapentin; as well as others, which are currently being developed (but may not yet be on the market; see e.g. http://www.newtbdrugs.org/pipeline.php). In particular, and as mentioned herein, compounds of the invention may be combined with one or more other inhibitors of the electron transport chain of mycobacteria, for instance a cytochrome bc inhibitor, an ATP synthase inhibitor, a NDH2 inhibitor and/or an inhibitor of the menaquinone synthesis pathway, such as a MenG inhibitor. Given that the compounds of the invention might act as cytochrome bd inhibitors, and hence target the electron transport chain of the mycobacteria (thereby blocking energy production of mycobacteria), the compounds of the invention (cytochrome bd inhibitors), combinations with one or more other inhibitors of the electron transport chain is thought to be a potentially effective way of providing an efficient regimen against mycobacteria. Even if the compounds of the invention (cytochrome bd inhibitors) alone might not be effective against mycobacteria, combining with one or more other such inhibitors may provide an effective regimen where the activity of one or more components of the combination is/are enhanced and/or such combinations act more effectively (e.g. synergistically).

### General Preparation

The compounds according to the invention can generally be prepared by a succession of steps, each of which may be known to the skilled person or described herein.

### Experimental Part

Compounds of formula I may be prepared in accordance with the techniques employed in the examples hereinafter (and those methods know by those skilled in the art), for example by using the following techniques.

Compounds of formula (I) may be prepared by:

(i) conversion of a compound of formula (II),

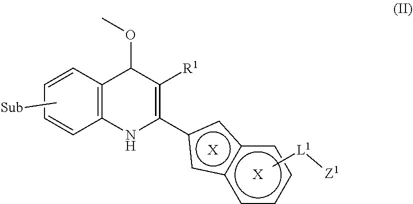

in which the integers are hereinbefore defined, by reaction with an appropriate such as BBr₃ or NaSCH₃ (for example, as described in the examples);

(ii) reaction of a compound of formula (III),

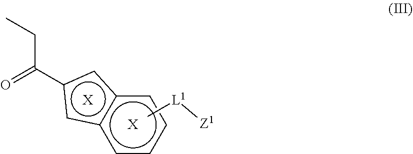

wherein the integers are as hereinbefore defined, with a compound of formula (IV),

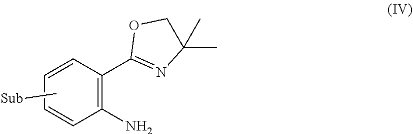

wherein the integers are hereinbefore defined, for example, in the presence of a reagent such as ZrCl₄, PTSA or the like, optionally in the presence of a solvent, such as an alcohol (e.g. butanol), under suitable reaction conditions (which may be further described in the examples).

It is evident that in the foregoing and in the following reactions, the reaction products may be isolated from the reaction medium and, if necessary, further purified according to methodologies generally known in the art, such as extraction, crystallization and chromatography. It is further evident that reaction products that exist in more than one enantiomeric form, may be isolated from their mixture by known techniques, in particular preparative chromatography, such as preparative HPLC, chiral chromatography. Individual diastereoisomers or individual enantiomers can also be obtained by Supercritical Fluid Chromatography (SCF).

The starting materials and the intermediates are compounds that are either commercially available or may be prepared according to conventional reaction procedures generally known in the art.

Experimental

Compounds of formula I may be prepared in accordance with the techniques employed in the examples hereinafter (and those methods know by those skilled in the art), for example by using the following techniques.

It is evident that in the foregoing and in the following reactions, the reaction products may be isolated from the reaction medium and, if necessary, further purified according to methodologies generally known in the art, such as extraction, crystallization and chromatography. It is further evident that reaction products that exist in more than one enantiomeric form, may be isolated from their mixture by known techniques, in particular preparative chromatography, such as preparative HPLC, chiral chromatography. Individual diastereoisomers or individual enantiomers can also be obtained by Supercritical Fluid Chromatography (SCF).

The starting materials and the intermediates are compounds that are either commercially available or may be prepared according to conventional reaction procedures generally known in the art.

Abbreviations

AcOH Acetic acid

BINAP R)-(+)-2,2′-Bis(diphenylphosphino)-1,1′-binaphthalene.

nBu₄NI Tetrabutylammonium iodide

BnBr Benzyl bromide

CAN/CH₃CN Acetonitrile

(CF₃CO)₂O Trifluoroacetic anhydride

Cs₂CO₃ Cesium carbonate

DEAD Diethyl azodicarboxylate

DCM or CH₂Cl₂ Dichloromethane

DMF Dimethylformamide

DMSO Methyl sulfoxide

Et₃N or TEA Triethylamine

EtOAc Ethyl acetate

EtOH Ethanol

FeCl₂ Iron(II) chloride tetrahydrate

h hour

H₂ Dihydrogen gas

HCl Hydrochloric acid

i-PrOH Isopropyl alcohol

iPrMgCl.LiCl Isopropylmagnesium chloride-Lithium chloride complex

K₂CO₃ Potassium carbonate

K₃PO₄.H₂O Potassium phosphate tribasic monohydrate

MeOH Methanol

MeTHF Methyltetrahydrofurane

MgSO₄ Magnesium sulfate

MSH O-Mesitylenesulfonylhydroxylamine

min Minute

N2 Nitrogen

NaBH(OAc)₃ Sodium triacetoxyborohydride

NaHCO₃ Sodium Bicarbonate

NaOH Sodium hydroxide

Na₂SO₄ Sodium sulfate

NH₂OH.HCl Hydroxylamine hydrochloride

NH₄Cl Ammonium, chloride

NMR Nuclear Magnetic Resonance

Pd/C Palladium on carbon

PddppfCl₂ [1,1′-Bis(diphenylphosphino)ferrocene]dichloropalladium(II)

Pd₂(dba)₃ Tris(dibenzylideneacetone)dipalladium(0)

PPA Polyphosphoric Acid

rt/RT Room temperature

THF Tetrahydrofurane

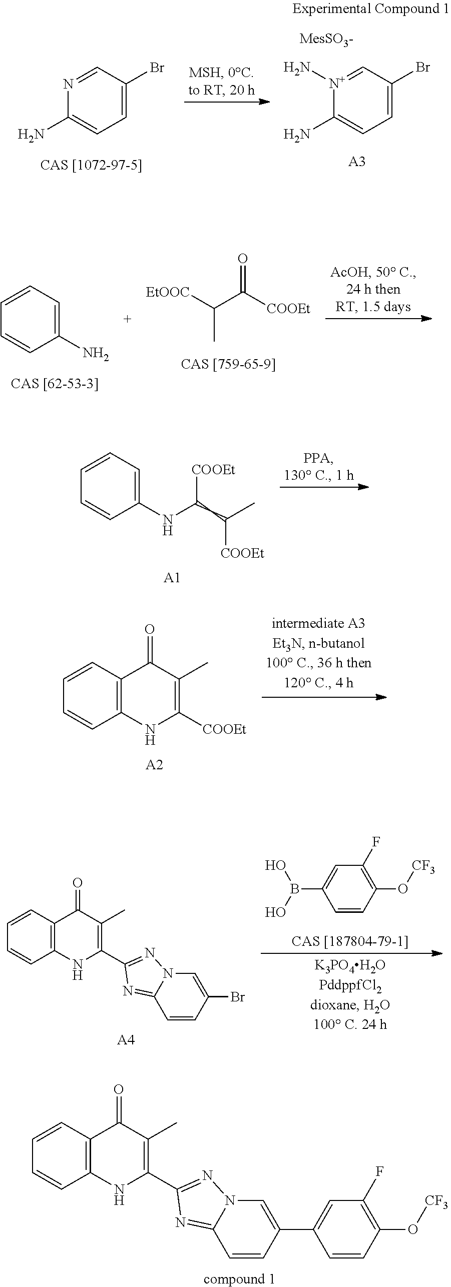

Preparation of Intermediate A1

To a mixture of diethyl oxalpropionate (CAS [759-65-9], 50.0 g, 247 mmol) and acetic acid (150 mL) was added aniline (CAS [62-53-3], 22.5 mL, 247 mmol) at room temperature. The resulting mixture was stirred at 50° C. for 24 h and at room temperature for 1.5 days. The reaction mixture was concentrated under reduced pressure and portioned between DCM (500 mL) and water (500 mL) and the aqueous layer was extracted with DCM (2×250 mL). The combined organic layers were dried over Na₂SO₄, filtered and concentrated to dryness under reduced pressure affording 68.3 g as an orange liquid. It was purified by flash chromatography over silica gel (cyclohexane/EtOAc 100/0 for 5 min, then 100/0 to 7/3 over 60 min) affording two fractions: 47.8 g (70% as a yellow liquid and 8.94 g (13%) as a yellow solid of intermediate A1.

Preparation of Intermediate A2

A mixture of intermediate A1 (46.5 g, 167 mmol) and polyphosphoric acid (304 g) was stirred at 130° C. for 1 h. The reaction mixture was poured onto ice water (800 mL). The aqueous layer was extracted with DCM (3×500 mL), the combined organic layers were washed with water (500 mL), a saturated NaHCO₃ solution (500 mL), dried over sodium sulfate, filtered and concentrated to dryness to afford intermediate A2 as a pale brown solid, 23.6 g (61%).

Preparation of Intermediate A3

To a crude solution of MSH (381 mL, max. 87.6 mmol) was added 2-amino-5-bromopyridine (CAS [1072-97-5], 7.58 g, 43.8 mmol) at 0° C. under nitrogen atmosphere. The resulting mixture was allowed to warm to room temperature and stirred for 20 h. The reaction mixture was filtered then the precipitate was washed with DCM (300 mL), dried under high vacuum (50° C., 4 h) to afford intermediate A3 as a white solid, 16.4 g (97%).

Preparation of Intermediate A4

To a solution of intermediate A3 (16.4 g, 42.3 mmol) in n-butanol (210 mL) were successively added triethylamine (17.7 mL, 127 mmol) and intermediate A2 (9.79 g, 42.3 mmol) at 0° C. The reaction mixture was stirred at 100° C. for 1.5 days then at 120° C. for 4 h. The reaction mixture was concentrated to dryness to a brown solid. The crude solid was purified by flash chromatography over silica gel (DCM/Acetone from 90/10 to 70/30 over 75 min) to give intermediate A4 as yellow solids, 5.39 g (36%).

Preparation of Compound 1

A mixture of intermediate A4 (300 mg, 0.845 mmol), 3-fluoro-4-(trifluoromethoxy) phenylboronic acid (CAS [187804-79-1], 227 mg, 1.01 mmol) and Potassium phosphate monohydrate (584 mg, 2.53 mmol) in 1,4-Dioxane (3.2 mL) and water (0.80 mL) was purged with argon (vacuum/argon: 3 times). [1,1′-Bis(diphenylphosphino)ferrocene] dichloropalladium(II) (61.8 mg, 84.5 μmol) was added and the reaction mixture was purged with argon (vacuum/argon: 3 times). The resulting mixture was stirred at 100° C. for 24 h. The reaction mixture was cooled to room temperature, diluted with water (50 mL), filtered through a glass frit to collect after rinsing with water (3×5 mL) a black solid, 0.41 g. It was purified by flash chromatography on silica gel (25 g), DCM/Methanol 100/0 to 98/2 over 50 min to afford an off-white solid, 0.311 g. It was triturated with methanol (2×3 mL) and dried under high vacuum at 50° C. (for 18 h) to afford Compound 1 as a white solid, 0.289 g, 75%.

¹H NMR (400 MHz, DMSO-d6) δ ppm 11.91 (s, 1H), 9.64-9.61 (m, 1H), 8.24 (dd, J=9.3 Hz, 1.8 Hz, 1H), 8.17-8.09 (m, 3H), 7.93 (d, J=8.3 Hz, 1H), 7.89-7.84 (m, 1H), 7.76 (t, J=8.0 Hz, 1H), 7.69-7.63 (m, 1H), 7.36-7.30 (m, 1H), 2.42 (s, 3H).

Preparation of Other Final Compounds

A mixture of intermediate A4 (1 eq.), boronic acid (1.2 eq.) and Potassium phosphate monohydrate (3 eq.) in 1,4-Dioxane (220 eq.) and water (260 eq.) was purged with nitrogen (vacuum/nitrogen: 3 times). [1,1′-Bis(diphenylphosphino)ferrocene] dichloropalladium(II) (0.15 eq.) was added and the reaction mixture was purged with nitrogen (vacuum/nitrogen: 3 times). The resulting mixture was stirred at 100° C. overnight. The solution was cooled down to room temperature. Water and DCM/MeOH (95/5) were added. The organic layer was separated, dried over MgSO₄, filtered and evaporated affording the crude mixture. Purification was carried out by flash chromatography over silica gel (24 g, irregular SiOH 25-40 μM, solid deposit on celite®, DCM/MeOH from 100/0 to 97/3). Pure fractions were collected and evaporated affording a pale beige powder of desired compound. It was triturated with DIPE and (e.g. a few drops) Heptane, the precipitate was filtered off and dried overnight under reduce pressure at 60° C. affording the final compound

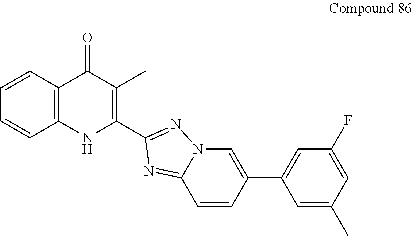 [SMILES: Cc1cc(F)cc(-c2ccc3nc(-c4[nH]c5ccccc5c(=O)c4C)nn3c2)c1]

Accordingly, compound 86 was prepared starting from intermediate A4 (0.39 mmol) and 3-Fluoro-5-methylphenyl boronic acid CAS [850593-06-5] yielding 0.15 g (69%) as white powder.

¹H NMR (500 MHz, DMSO-d₆) δ=11.90 (br s, 1H), 9.55 (br s, 1H), 8.02-8.38 (m, 3H), 7.92 (br d, J=7.5 Hz, 1H), 7.48-7.75 (m, 3H), 7.33 (br t, J=6.7 Hz, 1H), 7.14 (br d, J=8.7 Hz, 1H), 2.43 ppm (s, 3H), 2.41 (s, 3H)

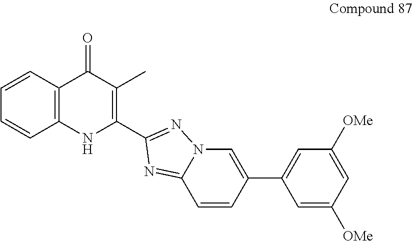 [SMILES: COc1cc(OC)cc(-c2ccc3nc(-c4[nH]c5ccccc5c(=O)c4C)nn3c2)c1]

Accordingly, compound 87 was prepared starting from intermediate A4 (0.56 mmol) and 3,5-dimethoxybenzene boronic acid CAS [192182-54-0] yielding 0.144 g (62%) as white powder.

¹H NMR (500 MHz, DMSO-d₆,) δ 11.89 (s, 1H), 9.54 (s, 1H), 8.21 (dd, J=1.5, 9.3 Hz, 1H), 8.15 (d, J=7.3 Hz, 1H), 8.07 (d, J=9.3 Hz, 1H), 7.93 (d, J=8.2 Hz, 1H), 7.66 (t, J=7.7 Hz, 1H), 7.33 (t, J=7.4 Hz, 1H), 7.03 (d, J=2.1 Hz, 2H), 6.5-6.6 (m, 1H), 3.86 (s, 6H), 2.43 (s, 3H)

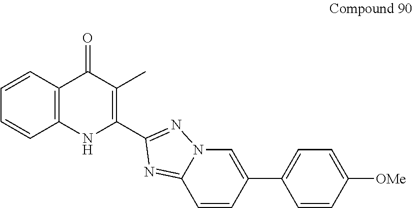 [SMILES: COc1ccc(-c2ccc3nc(-c4[nH]c5ccccc5c(=O)c4C)nn3c2)cc1]

Accordingly, compound 90 was prepared starting from intermediate A4 (0.56 mmol) and 4-methoxybenzene boronic acid CAS [5720-07-0] yielding 0.132 g (61%) as white powder.

¹H NMR (500 MHz, DMSO-d₆) δ 11.89 (br s, 1H), 9.40-9.43 (m, 1H), 8.13-8.18 (m, 2H), 8.06 (d, J=9.3 Hz, 1H), 7.92 (d, J=8.2 Hz, 1H), 7.83 (d, J=8.9 Hz, 2H), 7.66 (ddd, J=1.4, 6.9, 8.4 Hz, 1H), 7.33 (t, J=7.5 Hz, 1H), 7.11 (d, J=8.9 Hz, 2H), 3.83 (s, 3H), 2.42 (s, 3H)

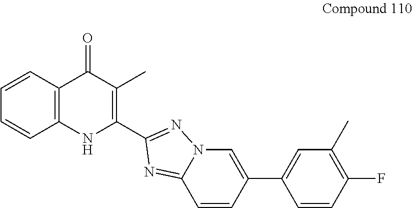 [SMILES: Cc1cc(-c2ccc3nc(-c4[nH]c5ccccc5c(=O)c4C)nn3c2)ccc1F]

Accordingly, compound 110 was prepared starting from intermediate A4 (1.35 mmol) and 4-Fluoro-3-methylbenzeneboronic acid CAS [139911-27-6] yielding 0.43 g (85%) as white powder.

¹H NMR (500 MHz, DMSO-d₆) δ 11.89 (br s, 1H), 9.47 (d, J=0.8 Hz, 1H), 8.13-8.19 (m, 2H), 8.08 (d, J=9.3 Hz, 1H), 7.93 (d, J=8.2 Hz, 1H), 7.86 (dd, J=7.3, 2.0 Hz, 1H), 7.72-7.77 (m, 1H), 7.66 (td, J=7.7, 1.6 Hz, 1H), 7.30-7.35 (m, 2H), 2.42 (s, 3H), 2.35 (d, J=1.4 Hz, 3H)

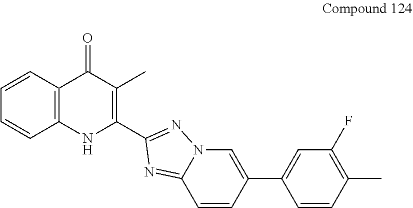 [SMILES: Cc1ccc(-c2ccc3nc(-c4[nH]c5ccccc5c(=O)c4C)nn3c2)cc1F]

Accordingly, compound 124 was prepared starting from intermediate A4 (1.18 mmol) and 3-Fluoro-4-methylbenzeneboronic acid CAS [168267-99-0] yielding 0.29 g (64%) as white powder.

¹H NMR (500 MHz, DMSO-d₆) δ 11.90 (br s, 1H), 9.54 (d, J=0.8 Hz, 1H), 8.22 (dd, J=1.8, 9.3 Hz, 1H), 8.14 (dd, J=1.2, 8.1 Hz, 1H), 8.08 (dd, J=0.7, 9.2 Hz, 1H), 7.93 (d, J=8.2 Hz, 1H), 7.75 (dd, J=1.7, 11.1 Hz, 1H), 7.63-7.69 (m, 2H), 7.46 (t, J=8.2 Hz, 1H), 7.33 (t, J=7.6 Hz, 1H), 2.42 (s, 3H), 2.31 (s, 3H)

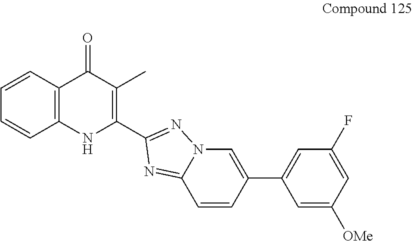 [SMILES: COc1cc(F)cc(-c2ccc3nc(-c4[nH]c5ccccc5c(=O)c4C)nn3c2)c1]

Accordingly, compound 125 was prepared starting from intermediate A4 (1.18 mmol) and 3-Fluoro-5-methoxyphenylboronic acid CAS [609807-25-2] yielding 0.34 g (72%) as white powder.

¹H NMR (500 MHz, DMSO-d₆) δ ppm 11.91 (s, 1H), 9.60 (d, J=0.8 Hz, 1H), 8.24 (dd, J=9.3, 1.8 Hz, 1H), 8.15 (dd, J=8.2, 1.2 Hz, 1H), 8.09 (dd, J=9.2, 0.7 Hz, 1H), 7.93 (d, J=8.2 Hz, 1H), 7.67 (ddd, J=8.4, 7.0, 1.5 Hz, 1H), 7.31-7.39 (m, 3H), 6.94 (dt, J=10.9, 2.2 Hz, 1H), 3.89 (s, 3H), 2.43 (s, 3H)

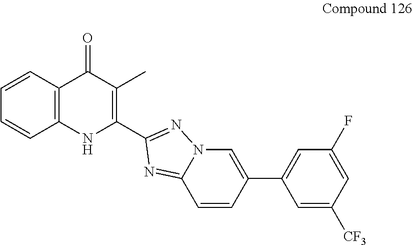 [SMILES: Cc1c(-c2nc3ccc(-c4cc(F)cc(C(F)(F)F)c4)cn3n2)[nH]c2ccccc2c1=O]

Accordingly, compound 126 was prepared starting from intermediate A4 (1.18 mmol) and 3-Fluoro-5-(trifluoromethyl)-benzene boronic acid CAS [159020-59-4] yielding 0.32 g (62%) as white powder.

¹H NMR (400 MHz, DMSO-d₆) δ ppm 11.92 (br s, 1H), 9.76 (s, 1H), 8.33 (dd, J=9.4, 1.7 Hz, 1H), 8.11-8.20 (m, 4H), 7.93 (d, J=8.4 Hz, 1H), 7.80 (br d, J=8.7 Hz, 1H), 7.66 (t, J=7.1Hz, 1H), 7.33 (t, J=7.5 Hz, 1H), 2.43 (s, 3H)

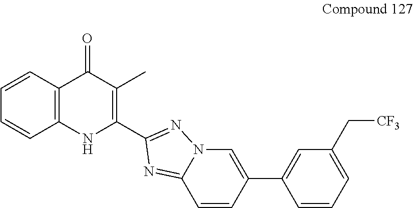 [SMILES: Cc1c(-c2nc3ccc(-c4cccc(CC(F)(F)F)c4)cn3n2)[nH]c2ccccc2c1=O]

Accordingly, compound 127 was prepared starting from intermediate A4 (1.35 mmol) and [3-(2,2,2)-trifluoroethyl)phenyl]-boronic acid CAS [1620056-82-7] yielding 0.54 g (91%) as white powder.

1H NMR (500 MHz, DMSO-d6) δ ppm 11.91 (br s, 1H), 9.49 (s, 1H), 8.09-8.21 (m, 3H), 7.85-7.97 (m, 3H), 7.67 (t, J=7.0 Hz, 1H), 7.58 (t, J=7.6 Hz, 1H), 7.48 (br d, J=7.5 Hz, 1H), 7.33 (t, J=7.6 Hz, 1H), 3.77 (q, J=11.3 Hz, 2H), 2.43 (s, 3H)

The following compounds are/were also prepared in accordance with the methods described herein:

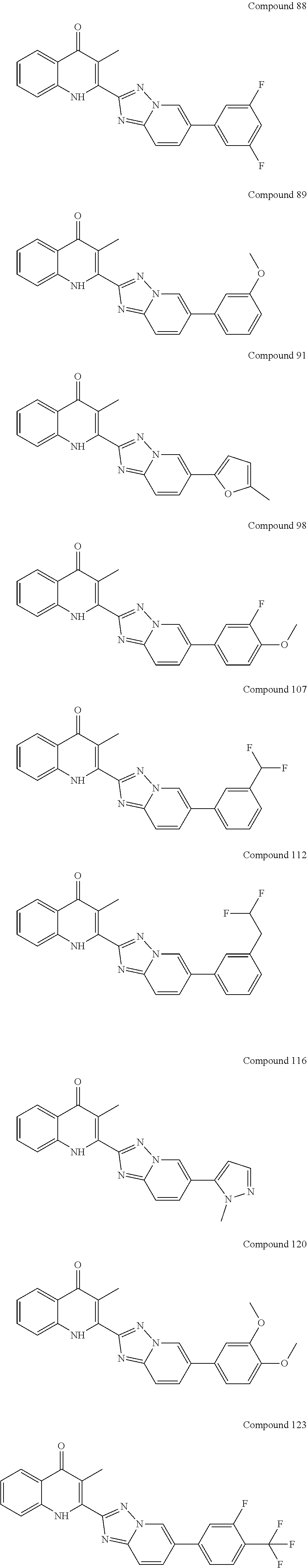

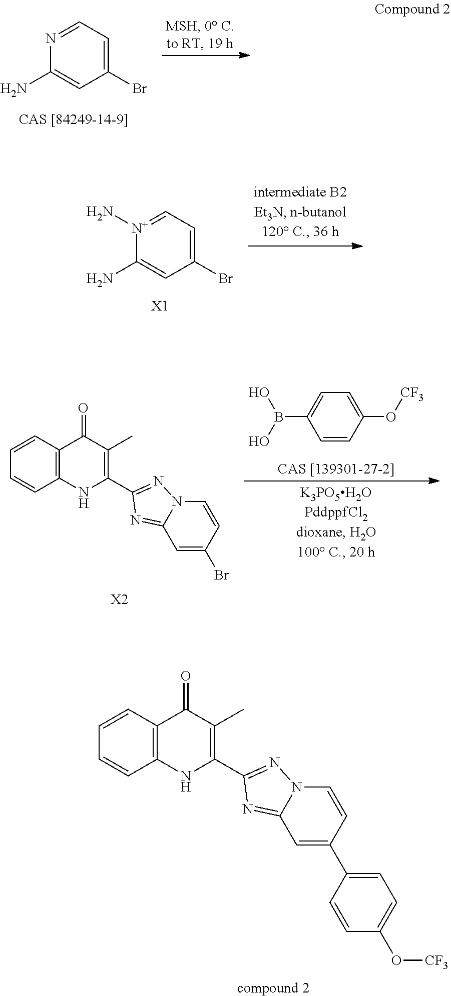

Preparation of Intermediate X1

To a crude solution of O-mesitylenesulfonylhydroxylamine (CAS [36016-40-7], 381 mL, max. 87.6 mmol) was added 2-amino-4-bromopyridine (CAS [84249-14-9], 12.6 g, 73.0 mmol) at 0° C. under nitrogen atmosphere. The resulting mixture was allowed to warm to room temperature and stirred for 18 h. The reaction mixture was filtered then the precipitate was washed with DCM (500 mL) to afford after high vacuum drying (60° C.) intermediate X1 as a white solid, 26.6 g, 94%.

Preparation of Intermediate X2

To a solution of intermediate X1 (26.6 g, 68.5 mmol) in n-butanol (340 mL) were successively added triethylamine (28.6 mL, 206 mmol) and intermediate B2 (15.8 g, 68.5 mmol). The reaction mixture was stirred at 120° C. for 1.5 days. The reaction mixture was concentrated to dryness to afford a brown solid. The crude solid was purified by flash chromatography over silica gel (DCM/Acetone 95/5 to 85/15 over 30 min then 85/15 to 80/20 over 30 min and 80/20 for 40 min) to give a yellow solid. It was dried under high vacuum at 50° C. (20 h) to afford intermediate X2 as a yellow solid, 2.1 g (9%).

Preparation of Compound 2

A mixture of intermediate X2 (2.02 g, 5.69 mmol), 4-trifluoromethoxyphenylboronic acid (CAS [139301-27-2], 1.41 g, 6.83 mmol) and potassium phosphate monohydrate (3.93 g, 17.1 mmol) in 1,4-dioxane (24 mL) and water (6 mL) was purged with argon. [1,1′-bis(diphenylphosphino)ferrocene] dichloropalladium (416 mg, 0.569 mmol) was then added and the resulting mixture was purged again with argon and stirred at 100° C. for 20 h. Water (˜50 mL) was added and the aqueous layer was filtered on a glass-frit to collect a black solid. This one was purified by column chromatography over silica gel (100/0 to 98/2 DCM/MeOH) to give a yellow solid, 3.35 g. It was triturated with MeOH (2×˜10 mL) to afford compound 2 as off-white solid, 1.74 g (70%).

1H NMR (400 MHz, DMSO-d₆) δ ppm 11.91 (s, 1H), 9.23 (d, J=7.2 Hz, 1H), 8.34-8.33 (m, 1H), 8.15 (dd, J=8.0 Hz, 1.0 Hz, 1H), 8.11-8.06 (m, 2H), 7.92 (d, J=8.4 Hz, 1H), 7.74 (dd, J=7.2 Hz, 1.9 Hz, 1H), 7.69-7.64 (m, 1H), 7.57 (d, J=8.3 Hz, 2H), 7.33 (t, J=7.5 Hz, 1H), 2.41 (s, 3H).

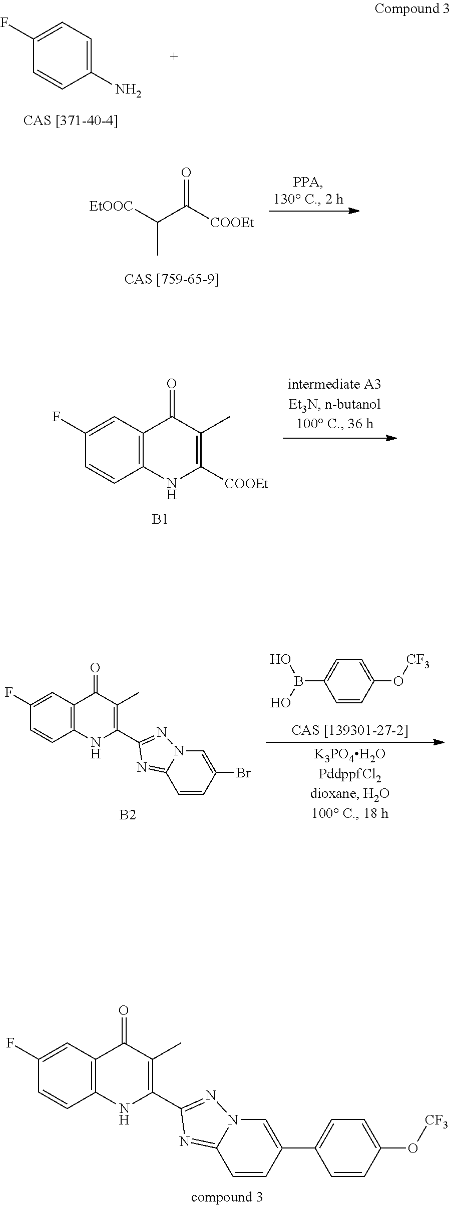

Preparation of Intermediate B1

To a mixture of diethyl oxalpropionate (CAS [759-65-9], 2.00 g, 9.89 mmol) and polyphosphoric acid (4.00 g) was added 4-fluoroaniline (CAS [371-40-4], 0.949 mL, 0.989 mmol) at room temperature. The resulting mixture was stirred at 130° C. for 2 h. The reaction mixture was poured onto ice water (50 mL). The aqueous layer was extracted with DCM (3×50 mL). The combined organic layers were washed with water (50 mL), a saturated aqueous NaHCO₃ solution (50 mL), dried over sodium sulfate, filtered and concentrated to dryness to afford a brownish sticky solid. It was triturated with diethyl ether (3×5 mL) and dried under reduced pressure to afford intermediate B1 as a pale-yellow solid, 0.565 g (23%).

Preparation of Intermediate B2

To a solution of intermediate A3 (862 mg, 2.22 mmol) and triethylamine (0.928 mL, 6.66 mmol) in n-butanol (11.1 mL) was added intermediate B1 (553 mg, 2.22 mmol) at 0° C. The resulting mixture was stirred at 100° C. for 18 h. The reaction mixture was concentrated to dryness and the residue was triturated with methanol (20 mL) collected on a glass frit and rinsed with methanol (3×10 mL) to afford intermediate B2 as a beige solid, 0.18 g (22%).

reparation of Compound 3

A mixture of intermediate B2 (175 mg, 0.469 mmol), 4-(trifluoromethoxy)phenylboronic acid (CAS [139301-27-2], 116 mg, 0.563 mmol) and Potassium phosphate monohydrate (324 mg, 1.41 mmol) in 1,4-dioxane (1.8 mL) and water (0.45 mL) was purged with argon (vacuum/argon: 3 times). [1,1′-Bis(diphenylphosphino)ferrocene] dichloropalladium(II) (34.3 mg, 46.9 μmol) was added and the reaction mixture was purged with argon (vacuum/argon: 3 times). The resulting mixture was stirred at 100° C. for 18 h. The reaction mixture was cooled to room temperature, diluted with water (25 mL), filtered through a glass frit to collect after rinsing with water (3×5 mL) a black solid. It was purified by flash chromatography on silica gel (25 g), DCM/Methanol 100/0 to 98/2 over 50 min) to afford an off-white solid. The solid was triturated with methanol (3×2 mL) and dried under high vacuum at 50° C. (for 18 h) to afford Compound 3 as a white solid, 0.107 g (50%).

¹H NMR (400 MHz, DMSO-d6) δ ppm 12.11 (s, 1H), 9.54 (s, 1H), 8.21 (dd, J=9.3 Hz, 1.7 Hz, 1H), 8.11 (d, J=9.3 Hz, 1H), 8.06-7.98 (m, 3H), 7.77 (dd, J=9.4 Hz, 2.9 Hz, 1H), 7.61 (td, J=8.8 Hz, 3.0 Hz, 1H), 7.55 (d, J=8.3 Hz, 2H), 2.44 (s, 3H).

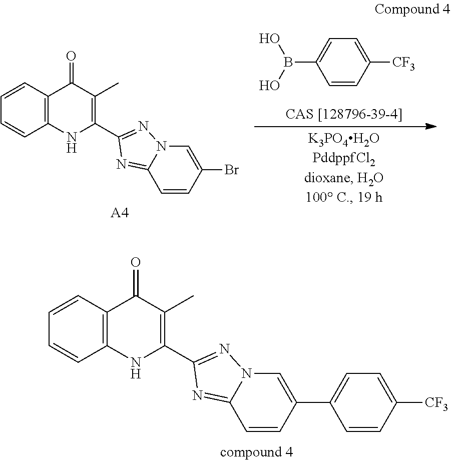

A mixture of intermediate A4 (300 mg, 0.845 mmol), 4-(trifluoromethyl)phenylboronic acid (CAS [128796-39-4], 193 mg, 1.01 mmol) and potassium phosphate monohydrate (584 mg, 2.53 mmol) in 1,4-dioxane (3.2 mL) and water (0.8 mL) was purged with argon. [1,1′-bis(diphenylphosphino)ferrocene] dichloropalladium (61.8 mg, 84.5 μmol) was then added and the resulting mixture was purged again with argon and stirred at 100° C. for 19 h. Water (50 mL) was added and the aqueous layers was filtered through a glass-frit to collect a black solid, 0.36 g. It was purified by column chromatography over silica gel (100/0 to 98/2 DCM/MeOH) to give a yellow solid, 0.235 g. This one was triturated with MeOH (2×2.5 mL) and dried under high vacuum at 50° C. (20 h) to afford Compound 4 as a pale-yellow solid, 0.21 g (59%).

¹H NMR (400 MHz, DMSO-d6) δ ppm 11.90 (s, 1H), 9.64-9.62 (m, 1H), 8.25 (dd, J=9.3 Hz, 1.8 Hz 1H), 8.17-8.10 (m, 4H), 7.95-7.89 (m, 3H), 7.69-7.64 (m, 1H), 7.36-7.31 (m, 1H), 2.43 (s, 3H).

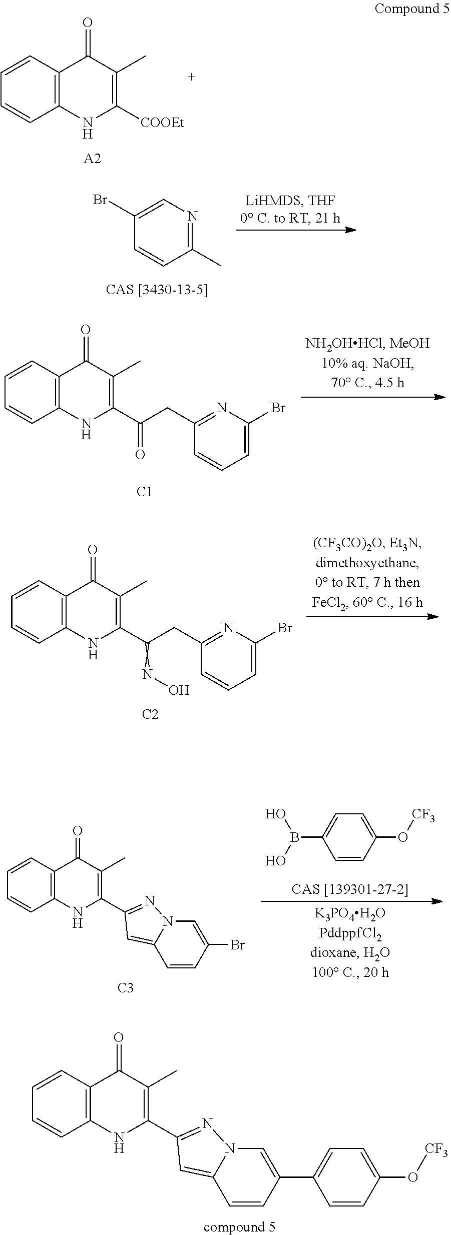

Preparation of Compound C1

To a solution of intermediate A2 (1.00 g, 4.32 mmol) and 5-bromo-2-methyl pyridine (CAS [3430-13-5], 0.744 g, 4.32 mmol) in D (10 mL) was added C (13.0 mL, 13.0 mmol) at 0° C. The resulting mixture was warm up to room temperature, stirred for 21 h and quenched with aq. sat NH₄Cl (50 mL). A yellow solid was filtrated on glass frit, washed with water (30 mL) and DCM (30 mL) and vacuum dried affording 0.984 g as a yellow solid. The combined filtrates were extracted with EtOAc (3×100 mL). The combined organic layers were washed with brine (50 mL), dried over Na₂SO₄, filtered and concentrated to dryness under reduced pressure affording intermediate C1, 0.365 g (24%) as an orange solid.

Preparation of Intermediate C2

To a solution of intermediate C1 (0.984 g, 2.76 mmol) in MeOH (22 mL) were added hydroxylamine hydrochloride (CAS [5470-11-1], 0.957 g, 13.8 mmol) and 10% aqueous solution of NaOH (8.92 mL, 24.8 mmol). The resulting mixture stirred at 70° C. for 4.5 h, then allowed to cool back to room temperature. The mixture was concentrated under reduced pressure to remove MeOH, then diluted with water (80 mL) and extracted with EtOAc (6×100 mL). The combined organic layers were dried over Na₂SO₄, filtered and concentrated to dryness under reduced pressure affording 0.724 g as a yellow solid. It was purified by flash chromatography over silica gel (DCM/MeOH from 100/0 to 95/5 over 25 min) affording intermediate C2, 0.459 g (45%) as a yellowish solid.

Preparation of Intermediate C3

To a solution of intermediate C2 (0.586 g, 1.57 mmol) in 1,2-dimethoxyethane (15 mL) was added trifluoroacetic anhydride (0.657 mL, 4.72 mmol) at 0° C. and the resulting mixture was stirred at 0° C. for 0.5 h. Then triethylamine (1.65 mL, 11.8 mmol) was added and the resulting mixture was stirred at room temperature for 7 h. Then iron(II) chloride (39.9 mg, 0.315 mmol) was added and the resulting mixture was stirred at 60° C. for 16 h. The mixture was diluted with water (30 mL) and extracted with DCM (3×50 mL). The combined organic layers were washed with aq. sat NaHCO₃ (50 mL), brine (50 mL), dried over Na₂SO₄, filtered and concentrated to dryness under reduced pressure affording 0.366 g as a brown solid. It was triturated with Et₂O (2×˜2 mL) and vacuum-dried affording 0.325 g (58%) of intermediate C3 as a brown solid.

Preparation of Compound 5

A mixture of intermediate C3 (0.160 g, 0.452 mmol), 4-Trifluoromethoxyphenylboronic acid (CAS [139301-27-2], 0.112 g, 0.542 mmol), Potassium phosphate monohydrate (0.312 g, 1.36 mmol) in a mixture of 1,4-dioxane (2 mL) and water (0.5 mL) was purged with argon before addition of [1,1′-Bis(diphenylphosphino)ferrocene]dichloropalladium (33.1 mg, 45.2 μmol). The resulting mixture was stirred at 100° C. for 16 h, then allowed to cool back to room temperature. Water (10 mL) was added to the reaction mixture and the precipitate was filtered on glass frit affording 0.166 g as a brown solid. This one was purified by flash chromatography over silica gel (DCM/MeOH from 100/0 to 95/5 in 25 min) affording a beige solid. The solid was triturated with Et₂O (2×˜2 mL) and vacuum-dried at 50° C. to give 0.106 g (54%) of Compound 5 as a white solid.

¹H NMR (400 MHz, DMSO-d6) δ ppm 11.68 (s, 1H), 9.24 (s, 1H), 8.14 (d, J=7.9 Hz, 1H), 8.00-7.94 (m, 3H), 7.79-7.72 (m, 2H), 7.67-7.61 (m, 1H), 7.52 (d, J=8.4 Hz, 2H), 7.31 (t, J=7.3 Hz, 1H), 7.17 (s, 1H), 2.21 (s, 3H).

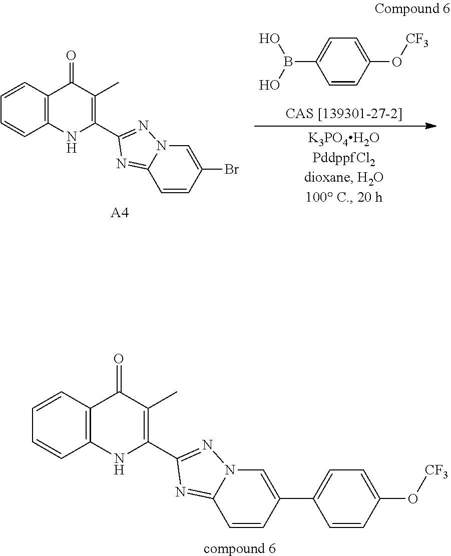

Preparation of Compound 6

A mixture of intermediate A4 (2.35 g, 6.62 mmol), 4-trifluoromethoxyphenylboronic acid (CAS [139301-27-2], 1.64 g, 7.94 mmol) and potassium phosphate monohydrate (4.57 g, 19.8 mmol) in 1,4-dioxane (28 mL) and water (7 mL) was purged with argon. [1,1′-bis(diphenylphosphino)ferrocene] dichloropalladium (484 mg, 0.662 mmol) was then added and the resulting mixture was purged again with argon and stirred at 100° C. for 20 h. Water (˜50 mL) was added and the aqueous layer was filtered to afford a grey solid. The aqueous layer was extracted with DCM (3×50 mL) and the combined organic layers were dried over sodium sulfate, filtered and concentrated to dryness to afford a black solid, 4.8 g. This one was purified by column chromatography over silica gel (100/0 to 95/5 DCM/MeOH) to give a beige solid, 3.12 g. The residue was triturated with MeOH (2×˜30 mL, collection by filtration) to afford after being dried under high vacuum at 50° C. (20 h) an off-white solid compound 6, 2.15 g (75%).

¹H NMR (400 MHz, DMSO-d6) δ ppm 11.88 (s, 1H), 9.54 (dd, J=1.8 Hz, 0.9 Hz, 1H), 8.20 (dd, J=9.3 Hz, 1.9 Hz, 1H), 8.15 (dd, J=8.2 Hz, 1.1 Hz, 1H), 8.11 (dd, J=9.4 Hz, 0.9 Hz, 1H), 8.04-8.00 (m, 2H), 7.93 (d, J=8.2 Hz, 1H), 7.69-7.64 (m, 1H), 7.58-7.53 (m, 2H), 7.36-7.30 (m, 1H), 2.43 (s, 3H)

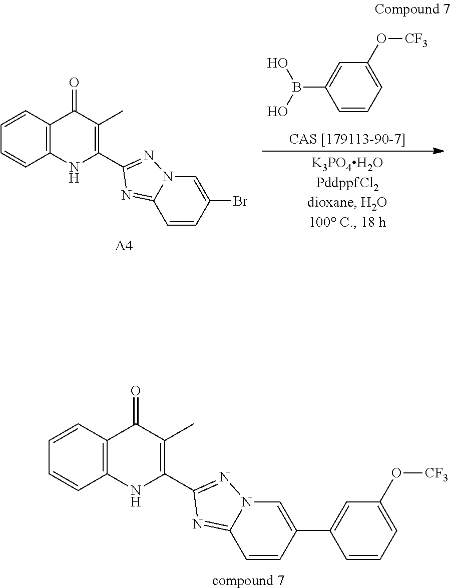

A mixture of intermediate A4 (300 mg, 0.845 mmol), 3-trifluoromethoxyphenylboronic acid (CAS [179113-90-7], 209 mg, 1.01 mmol) and potassium phosphate monohydrate (584 mg, 2.53 mmol) in 1,4-dioxane (3.2 mL) and water (0.8 mL) was purged with argon. [1,1′-bis(diphenylphosphino)ferrocene] dichloropalladium(II) (61.8 mg, 0.0845 mmol) was then added and the resulting mixture was purged again with argon and stirred at 100° C. for 18 hours. Water (50 mL) was added and the resulting precipitate was collected by filtration on a glass-frit and washed with water (30 mL) to afford a black solid, 0.424 g. This one was purified by flash chromatography over silica gel (from 0 to 4% of MeOH in DCM over 45 min). The desired collected fractions were concentrated under reduced pressure and the resulting solid was triturated with MeOH (3×2 mL) and vacuum-dried at 60° C. for 72 h to afford Compound 7 as a beige solid, 0.277 g (75%).

¹H NMR (400 MHz, DMSO-d6) δ ppm 11.90 (s, 1H), 9.62 (s, 1H), 8.24 (dd, J=9.3 Hz, 1.7 Hz, 1H), 8.15 (d, J=7.6 Hz, 1H), 8.11 (d, J=9.3 Hz, 1H), 7.98-7.91 (m, 3H), 7.72-7.63 (m, 2H), 7.48 (d, J=8.2 Hz, 1H), 7.33 (t, J=7.4 Hz, 1H), 2.43 (s, 3H).

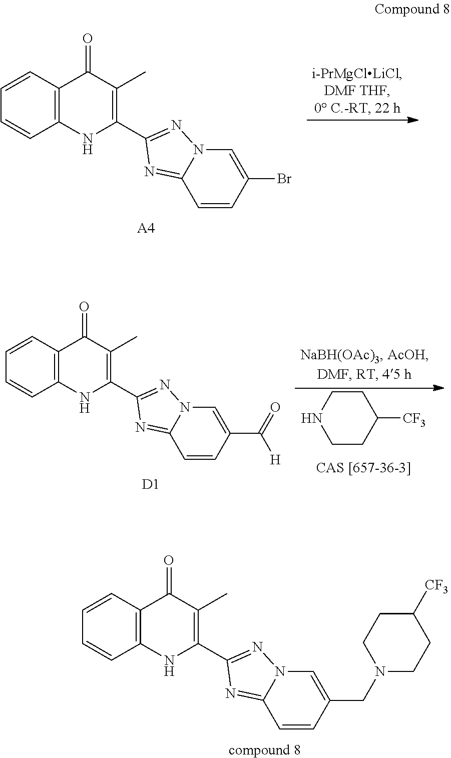

Preparation of Intermediate D1

A 1.3 M solution of isopropylmagnesium chloride lithium chloride complex in THF (6.50 ml, 8.45 mmol) was added dropwise to a solution of intermediate A4 (1.00 g, 2.82 mmol) in THF (7 ml) at 0° C. under argon atmosphere. The resulting mixture was stirred at 0° C. for 5 min and at room temperature for 2 h, then cooled again to 0° C. and DMF (0.327 ml, 4.22 mmol) was added. The resulting mixture was stirred at room temperature for 20 h, then quenched with a saturated aqueous NH₄Cl solution and extracted with a CH₂Cl₂/MeOH (9:1) mixture. The combined organic layers were dried over Na₂SO₄, filtered and concentrated under reduced pressure. The residue was roughly purified by flash chromatography on silica gel (CH₂Cl₂/EtOAc from 100:0 to 0:100) to afford D1 as a light yellow solid (0.523 g, purity ˜50%, yield 31%) which was used as such for the next step.

Preparation of Compound 8

To an argon-purged mixture of D1 as obtained in the previous step (purity ˜50%, 271 mg, 0.445 mmol) in DMF (8 ml) was added 4-(trifluoromethyl)piperidine (CAS [657-36-3], 0.136 g, 0.891 mmol). The solution was stirred at room temperature for 1 h followed by addition of AcOH (0.5 ml) and then portionwise (in the course of ˜5 min) NaBH(OAc)₃ (236 mg, 1.11 mmol). The resulting mixture was stirred at room temperature for 3.5 h, then concentrated under reduced pressure, diluted with a saturated aqueous NaHCO₃ solution and extracted with a CH₂Cl₂/MeOH (9:1) mixture. The combined organic layers were dried over Na₂SO₄, filtered and concentrated under reduced pressure. The residue was purified by flash chromatography on silica gel (CH₂Cl₂/MeOH from 100:0 to 95:5) and vacuum dried (60° C., 20 h) to afford compound 8 as a white solid (69 mg, 35%).

¹H NMR (400 MHz, DMSO-d₆) δ ppm 11.83 (s, 1H), 9.04 (s, 1H), 8.14 (d, J=8.0 Hz, 1H), 7.97 (d, J=9.2 Hz, 1H), 7.92 (d, J=8.4 Hz, 1H), 7.79 (dd, J=9.1 Hz, 1.2 Hz, 1H), 7.68-7.62 (m, 1H), 7.32 (t, J=7.6 Hz, 1H), 3.66 (s, 2H), 2.96 (br d, J=11.5 Hz, 2H), 2.40 (s, 3H), 2.35-2.22 (m, 1H), 2.12-2.02 (m, 2H), 1.80 (br d, J=12.2 Hz, 2H), 1.48 (qd, J=12.4 Hz, 3.8 Hz, 2H).

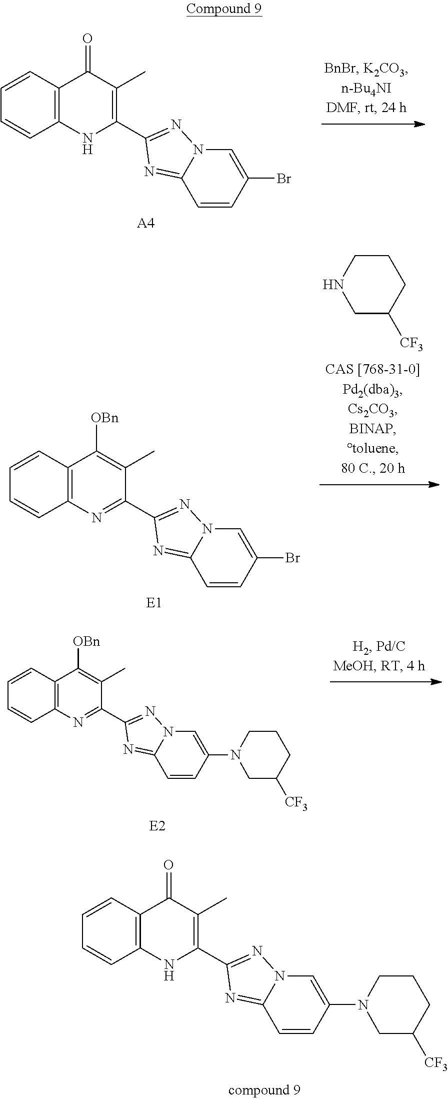

Preparation of Intermediate E1

A mixture of A4 (1.50 g, 4.22 mmol), benzyl bromide (0.603 ml, 5.07 mmol), K₂CO₃ (1.75 g, 12.7 mmol) and tetra-n-butylammonium iodide (0.312 g, 0.845 mmol) in DMF (28 ml) was stirred at room temperature for 24 h under argon atmosphere, then diluted with water and extracted with EtOAc. The combined organic layers were washed with brine, dried over Na₂SO₄, filtered and concentrated under reduced pressure. Purification by flash chromatography over silica gel (CH₂Cl₂/MeOH from 100:0 to 97:3) and re-purification by flash chromatography over silica gel (CH₂Cl₂/acetone from 100:0 to 60:40) afforded E1 as a beige solid (1.31 g, 70%).

Preparation of Intermediate E2

To an argon-purged mixture of E1 (250 mg, 0.561 mmol), 3-(trifluoromethyl)piperidine (CAS [768-31-0], 89.4 μl, 0.674 mmol) and Cs₂CO₃ (549 mg, 1.68 mmol) in toluene (3.7 ml) were added Pd₂(dba)₃ (77.1 mg, 0.0842 mmol) and rac-BINAP (105 mg, 0.168 mmol). The resulting mixture was purged again with argon and stirred at 80° C. for 20 h, then concentrated under reduced pressure and diluted with water. The resulting precipitate was collected by filtration on a glass-frit, washed with water and purified by flash chromatography over silica gel (CH₂Cl₂/acetone from 100:0 to 40:60) to afford E2 as a brownish solid (105 mg, 36%).

Preparation of Compound 9

A mixture of E2 (177 mg, 0.342 mmol) in MeOH (3.4 ml) was stirred in the presence of 10 wt % palladium on carbon (36.4 mg, 0.0342 mmol) under hydrogen atmosphere (1 atm.) at room temperature for 4 h. The reaction mixture was diluted with CH₂Cl₂ and filtered through a pad of Celite®. The filter cake was rinsed with CH₂Cl₂ and the filtrate was concentrated under reduced pressure. The residue was purified by flash chromatography over silica gel (CH₂Cl₂/MeOH from 100:0 to 98:2) to afford after co-evaporation with MeOH and vacuum-drying (60° C., 48 h) compound 9 as a beige solid (51.8 mg, 35%).

¹H NMR (400 MHz, DMSO-d₆) δ ppm 11.77 (s, 1H), 8.58 (s, 1H), 8.13 (dd, J=8.2 Hz, 1.3 Hz, 1H), 7.90 (d, J=8.3 Hz, 1H), 7.88-7.81 (m, 2H), 7.67-7.60 (m, 1H), 7.34-7.27 (m, 1H), 3.83 (br d, J=11.4 Hz, 1H), 3.70 (br d, J=12.4 Hz, 1H), 2.87-2.65 (m, 3H), 2.39 (s, 3H), 2.04-1.96 (m, 1H), 1.90-1.82 (m, 1H), 1.77-1.64 (m, 1H), 1.47 (qd, J=12.2 Hz, 4.0 Hz, 1H).

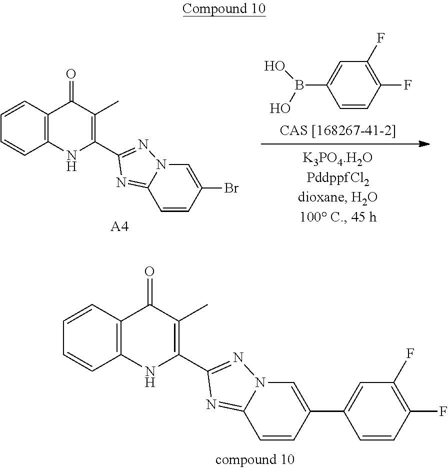 [SMILES: Cc1c(-c2nc3ccc(Br)cn3n2)[nH]c2ccccc2c1=O ; OB(O)c1ccc(F)c(F)c1 ; "A4" ; Cc1c(-c2nc3ccc(-c4ccc(F)c(F)c4)cn3n2)[nH]c2ccccc2c1=O]

To a nitrogen purged-mixture of intermediate A4 (300 mg, 0.845 mmol), 3,4-difluorophenylboronic acid (CAS [168267-41-2], 213 mg, 1.35 mmol, 1.6 eq.) and potassium phosphate monohydrate (389 mg, 1.69 mmol, 2eq.) in a mixture of 1,4-dioxane (4.8 mL) and water (1.2 mL) was added [1,1′-Bis(diphenylphosphino)ferrocene] dichloropalladium(II) (124 mg, 0.169 mmol, 0.2 eq.). This mixture was purged again with argon and then stirred at 100° C. for 21 h. The reaction mixture was cooled to room temperature before the addition of 3,4-Difluorophenylboronic acid (66.7 mg, 0.422 mmol, 0.5 eq.) and Potassium phosphate monohydrate (195 mg, 0.845 mmol, 1 eq.). This mixture was purged with nitrogen and then [1,1′-Bis(diphenylphosphino)ferrocene] dichloropalladium(II) (61.8 mg, 0.084 mmol, 0.1 eq.) was added. This mixture was purged again with nitrogen and then stirred at 100° C. for 24 h. The reaction mixture was cooled to rt, diluted with water (25 mL) and filtered through a glass-frit. The resulting residue was washed with water (3×25 mL) and dried under vacuum for 2 h to afford a black solid, 0.451 g. The crude was purified by flash chromatography over silica gel (0 to 4% MeOH in DCM over 30 min and then 4% MeOH over 30 min) to afford a brown solid, 0.228 g. It was purified by flash chromatography over silica gel (from 0 to 10% of a mixture toluene/MeOH (7:3) in DCM over 80 min) to afford 0.2 g. This one was triturated with MeOH (3×2 mL). A suspension of the resulting solid in MeOH (15 mL) was heated at 70° C. for 5 h. The mixture was cooled to room temperature and the resulting solid was collected by filtration and dried under high-vacuum at 60° C. for 3 days to afford Compound 10 as a beige solid, 0.093 g (28%).

¹H NMR (400 MHz, DMSO-d6) δ ppm 11.90 (s, 1H), 9.57 (s, 1H), 8.22 (dd, J=9.3 Hz, 1.7 Hz, 1H), 8.17-8.02 (m, 3H), 7.93 (d, J=8.3 Hz, 1H), 7.81-7.74 (m, 1H), 7.70-7.58 (m, 2H), 7.33 (t, J=7.5 Hz, 1H), 2.42 (s, 3H).

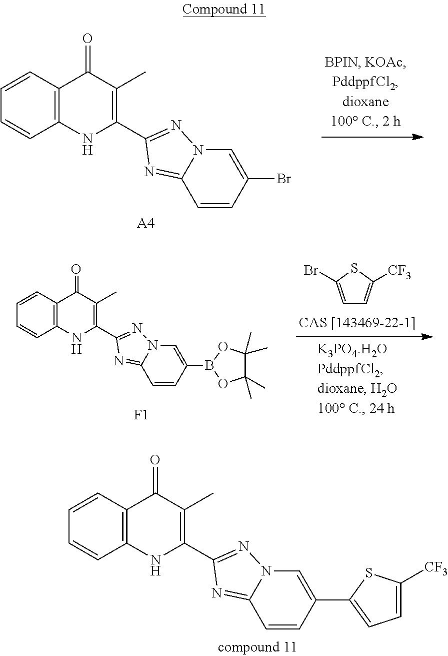

Preparation of Intermediate F1

A nitrogen atmosphere purged mixture of intermediate A4 (1.00 g, 2.82 mmol), bis(pinacolato)diboron (CAS [73183-34-3], 858 mg, 3.38 mmol), potassium acetate (691 mg, 7.04 mmol) and [1,1′-bis(diphenylphosphino)ferrocene]dichloropalladium (206 mg, 0.282 mmol) in 1,4-dioxane (14 mL) was stirred at 100° C. for 2 h. The mixture was concentrated under reduced pressure and the residue directly purified by flash chromatography over silica gel (DCM/Acetone 100/0 to 0/100 30 min) affording a light brown solid. It was triturated in n-pentane (3×5 mL), filtered off. The solid was triturated in Et₂O (3×5 mL) and vacuum-dried affording compound F1 as a white solid 0.339 g (30%).

Preparation of Compound 11

An argon-purged mixture of intermediate F1 (200 mg, 0.497 mmol), 2-bromo-5-(trifluoromethyl)thiophene (CAS [143469-22-1], 172 mg, 0.746 mmol), K₃PO₄.H₂O (343 mg, 1.49 mmol), Pd(dppf)Cl₂ (109 mg, 0.149 mmol) in 1,4-dioxane (3.8 ml) and water (1.3 ml) was stirred at 100° C. for 24 h. The reaction mixture was cooled back to room temperature, diluted with water (20 ml) and extracted with a CH₂Cl₂/MeOH (1:1) mixture. The combined organic layers were washed with brine, dried over Na₂SO₄, filtered and concentrated under reduced pressure. The crude residue was purified by flash chromatography over silica gel (CH₂Cl₂/MeOH from 100:0 to 95:5) followed by subsequent successive trituration with MeOH, CH₂Cl₂/MeOH (8:2) and acetonitrile. Vacuum-drying (40° C., 3 h and 60° C., 20 h) afforded compound 11 as a white solid (124 mg, 58%).

¹H NMR (400 MHz, DMSO-d6) δ ppm 11.90 (s, 1H), 9.71 (s, 1H), 8.19 (dd, J=9.3 Hz, 1.7 Hz, 1H), 8.15 (dd, J=8.3 Hz, 1.0 Hz, 1H), 8.11 (d, J=9.3 Hz, 1H), 7.94-7.85 (m, 3H), 7.69-7.63 (m, 1H), 7.36-7.30 (m, 1H), 2.42 (s, 3H).

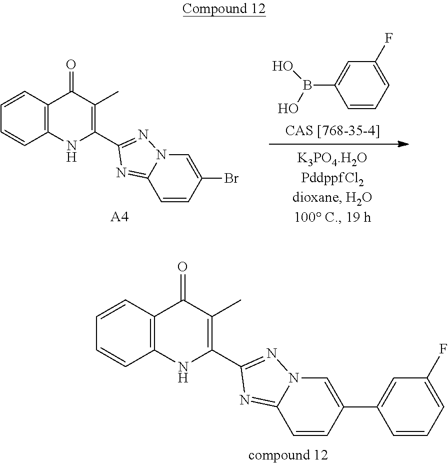 [SMILES: Cc1c(-c2nc3ccc(Br)cn3n2)[nH]c2ccccc2c1=O ; OB(O)c1cccc(F)c1 ; "A4" ; Cc1c(-c2nc3ccc(-c4cccc(F)c4)cn3n2)[nH]c2ccccc2c1=O]

A mixture of intermediate A4 (300 mg, 0.845 mmol), 3-fluorophenylboronic acid (CAS [768-35-4], 142 mg, 1.01 mmol) and potassium phosphate monohydrate (584 mg, 2.53 mmol) in a mixture of 1,4-dioxane (3.2 mL) and water (0.8 mL) was purged with argon. [1,1′-Bis(diphenylphosphino)ferrocene] dichloropalladium(II) (61.8 mg, 0.0845 mmol) was then added and the resulting mixture was purged again with argon and stirred at 100° C. for 19 h. Water (50 mL) was added and the resulting precipitate was collected by filtration on a glass-frit and washed with water (30 mL) to afford a black solid, 0.312 g. It was purified by flash chromatography over silica gel (from 0 to 5% of MeOH in DCM over 1.05 h). The desired collected fractions were concentrated under reduced pressure and the resulting solid was triturated with MeOH (3×2 mL) and vacuum-dried at 60° C. for 48 h to afford Compound 12 as a beige solid, 0.230 g (73%).

¹H NMR (400 MHz, DMSO-d6) δ ppm 11.90 (s, 1H), 9.58 (s, 1H), 8.24 (dd, J=9.2 Hz, 1.6 Hz, 1H), 8.15 (d, J=8.1 Hz, 1H), 8.10 (d, J=9.4 Hz, 1H), 7.93 (d, J=8.4 Hz, 1H), 7.83-7.78 (m, 1H), 7.76 (d, J=7.9 Hz, 1H), 7.69-7.63 (m, 1H), 7.63-7.56 (m, 1H), 7.36-7.28 (m, 2H), 2.43 (s, 3H).

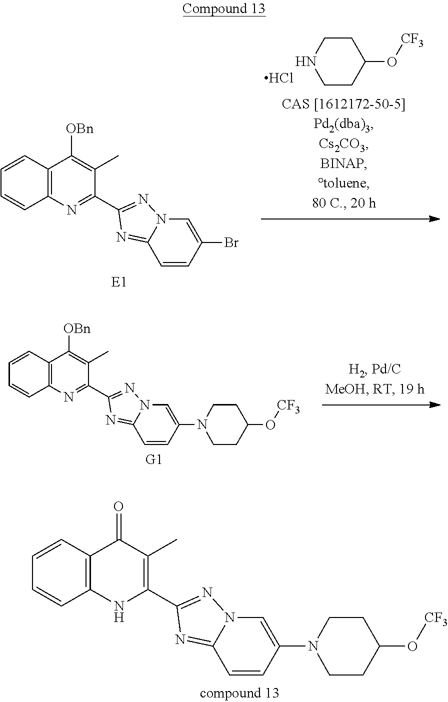

Preparation of Intermediate G1

To an argon-purged mixture of E1 (250 mg, 0.561 mmol), 4-(trifluoromethoxy)piperidine hydrochloride (CAS [1612172-50-5], 139 mg, 0.674 mmol) and Cs₂CO₃ (732 mg, 2.25 mmol) in toluene (3.7 ml) were added Pd(OAc)₂ (25.2 mg, 0.112 mmol) and rac-BINAP (69.9 mg, 0.112 mmol). The resulting mixture was purged again with argon and stirred at 80° C. for 24 h, then concentrated under reduced pressure and partitioned between CH₂Cl₂ and water. The aqueous layer was further extracted with CH₂Cl₂ and the combined organic layers were washed with brine, dried over Na₂SO₄, filtered and concentrated under reduced pressure. The residue was purified by flash chromatography over silica gel (CH₂Cl₂/EtOAc from 100:0 to 0:100) and in part re-purified by flash chromatography over silica gel (CH₂Cl₂/acetone from 100:0 to 50:50). The purest fractions of these 2 purifications were combined and re-purified by flash chromatography over silica gel (CH₂Cl₂/MeOH from 100:0 to 90:10) to afford G1 as a brownish solid (72.6 mg, 24%).

Preparation of Compound 13

A mixture of G1 (102 mg, 0.191 mmol) in MeOH (2 ml) was stirred in the presence of 10 wt % palladium on carbon (20.3 mg, 0.0191 mmol) under hydrogen atmosphere (1 atm.) at room temperature for 19 h. The reaction mixture was diluted with CH₂Cl₂ and filtered through a pad of Celite®. The filter cake was rinsed with CH₂Cl₂/MeOH (9:1) and the filtrate was concentrated under reduced pressure. The residue was purified by flash chromatography over silica gel (CH₂Cl₂/MeOH from 100:0 to 95:5) to afford after trituration with MeOH and vacuum-drying (60° C., 24 h) compound 13 as a pale grey solid (46.9 mg, 55%).

¹H NMR (400 MHz, DMSO-d₆) δ ppm 11.77 (s, 1H), 8.53 (d, J=1.4 Hz, 1H), 8.13 (dd, J=8.3 Hz, 1.0 Hz, 1H), 7.90 (d, J=8.4 Hz, 1H), 7.88-7.80 (m, 2H), 7.67-7.61 (m, 1H), 7.33-7.28 (m, 1H), 4.72-4.65 (m, 1H), 3.57-3.49 (m, 2H), 3.19-3.10 (m, 2H), 2.39 (s, 3H), 2.14-2.05 (m, 2H), 1.92-1.82 (m, 2H).

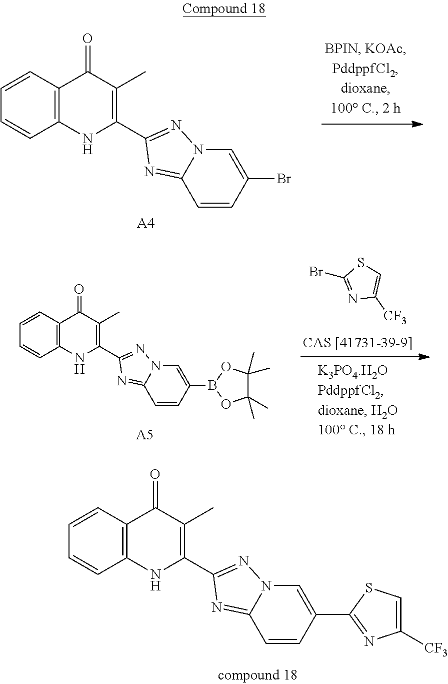

Preparation of Intermediate A5

A nitrogen atmosphere purged mixture of intermediate A4 (1.00 g, 2.82 mmol), bis(pinacolato)diboron (CAS [73183-34-3], 858 mg, 3.38 mmol), potassium acetate (691 mg, 7.04 mmol) and [1,1′-bis(diphenylphosphino)ferrocene]dichloropalladium (206 mg, 0.282 mmol) in 1,4-dioxane (14 mL) was stirred at 100° C. for 2 h. The mixture was concentrated under reduced pressure and the residue directly purified by flash chromatography over silica gel (cartridge Interchim IR-50SI-F0050, DCM/Acetone 100/0 to 0/100 30 min) affording a light brown solid. It was triturated in n-pentane (3×5 mL), filtered off. The solid was triturated in Et₂O (3×5 mL) and vacuum-dried affording compound D1 as a white solid 0.339 g (30%).

Preparation of Compound 18

An argon-purged mixture of intermediate A5 (150 mg, 0.373 mmol), 2-bromo-4-(trifluoromethyl)thiazole (CAS [41731-39-9], 86.5 mg, 0.373 mmol), potassium phosphate monohydrate (258 mg, 1.12 mmol), [1,1′-bis(diphenylphosphino)ferrocene] dichloropalladium (27.3 mg, 0.037 mmol) in 1,4-dioxane (1.5 mL) and water (0.3 mL) was stirred at 100° C. for 18 h. The reaction mixture was cooled to room temperature, diluted with water (5 mL) and the solid was collected by filtration on a glass frit affording a grey solid. The solid was then purified by flash chromatography (cartridge Interchim IR-50SI-F0025, DCM/MeOH from 100/0 to 95/5 in 30 min) affording a brownish solid. It was recrystallized in MeOH (3 mL) affording a white solid and was dried in vacuum (60° C., 60 h) affording Compound 18, 0.064 g (40%).

1H NMR (400 MHz, DMSO-d6) δ ppm 11.92 (s, 1H), 9.88 (s, 1H), 8.69 (s, 1H), 8.38 (dd, J=9.3 Hz, 1.7 Hz, 1H), 8.17-8.13 (m, 2H), 7.93 (d, J=8.3 Hz, 1H), 7.70-7.64 (m, 1H), 7.34 (t, J=7.5 Hz, 1H), 2.42 (s, 3H).

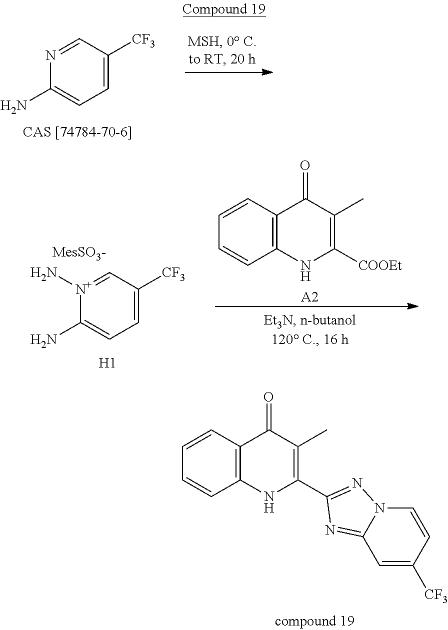

Preparation of Intermediate H1

Accordingly, intermediate H1 was prepared in the same way as intermediate A3, starting from 2-amino-5-trifluoromethylpyridine (CAS[74784-70-6], 11 mmol). Intermediate H1 was obtained as a white solid, 1.71 g (41%).

Preparation of Compound 19

To a solution of intermediate H1 (1.55 g, 4.11 mmol) in n-butanol(24 ml) were added triethylamine (2.86 ml, 20.5 mmol) and intermediate A2 (0.950 g, 4.11 mmol) and the resulting mixture was stirred at 120° C. for 16 hours, then allowed to cool back to room temperature. The mixture was concentrated to dryness under reduced pressure affording 3.14 g as a brown gum.

This one was purified by flash chromatography over silica gel (DCM/acetone from 95/5 to 85/15) affording 0.339 g as a yellow solid. It was triturated with MeOH (˜3 ml), filtered off and vacuum-dried (50° C., 17 h) affording compound 19 as a pale yellow solid, 0.259 g (18%)

¹H NMR (400 MHz, DMSO-d6) δ ppm 11.92 (s, 1H), 9.87 (s, 1H), 8.22 (d, J=9.4 Hz, 1H), 8.15 (dd, J=8.1 Hz, 1.4 Hz, 1H), 8.11 (d, J=9.4 Hz, 1.7 Hz, 1H), 7.91 (d, J=8.3 Hz, 1H), 7.69-7.64 (m, 1H), 7.36-7.31 (m, 1H), 2.40 (s, 3H).

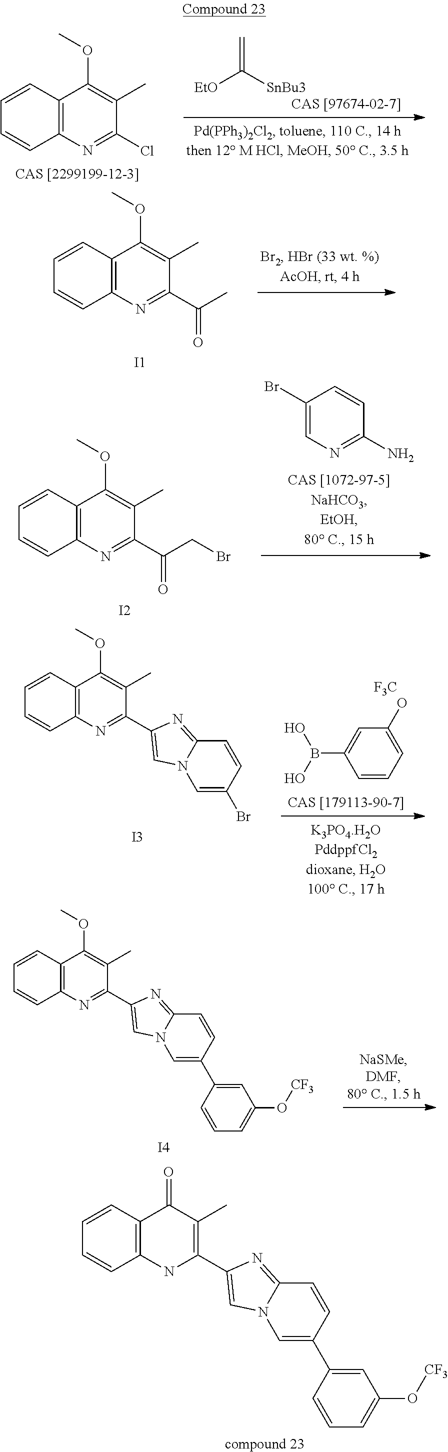

Preparation of Intermediate I1

A solution of 2-chloro-4-methoxy-3-methyl-quinoline (CAS [2299199-12-3], 3.00 g, 14.4 mmol) and tributyl(1-ethoxyvinyl)tin (CAS [97674-02-7], 6.35 mL, 18.8 mmol) in toluene (60 mL) was argon-purged bis(triphenylphosphine)palladium(II) dichloride (0.507 g, 0.722 mmol) was added and the mixture was purged again with argon and stirred at 110° C. for 14 h. The reaction mixture was concentrated under reduced pressure to approximately 15 mL, then MeOH (60 mL) and a 12 M aqueous solution of HCl (15 mL) were added and the mixture was stirred at 50° C. for 3.5 h. MeOH was removed under reduced pressure and 3 M aqueous NaOH was added until pH ˜7. The aqueous layer was extracted with CH₂Cl₂ and the combined organic layers were dried over Na₂SO₄ and concentrated to dryness. The residue was purified by flash chromatography over silica gel (cyclohexane/EtOAc 95:5) to afford intermediate I1 as a white solid (2.09 g, 64%).

Preparation of Intermediate I2

To a solution of intermediate intermediate I1 (2.09 g, 9.20 mmol) in AcOH (40 mL) were added successively HBr 33 wt. % in acetic acid (6.50 mL, 37.1 mmol) and bromine (0.498 mL, 9.66 mmol) and the mixture was stirred at room temperature for 4 h. The reaction mixture was concentrated to dryness, then the residue was taken up with CH₂Cl₂ and a saturated aqueous solution of NaHCO₃ and the aqueous layer was extracted with CH₂Cl₂. The combined organic layers were dried over sodium sulfate, filtered and concentrated to dryness. The crude product intermediate I2 was considered as quantitative and used as such in the next step (2.84 g containing maximum 9.20 mmol).

Preparation of Intermediate I3

To a solution of crude intermediate I2 (0.500 g, max. 1.54 mmol) in EtOH (16 mL) were added 2-amino-5-bromopyridine (CAS [1072-97-5], 0.267 g, 1.54 mmol) and NaHCO₃ (0.259 g, 3.08 mmol). The resulting mixture was stirred at 80° C. for 15 h. The reaction mixture was combined with another reaction mixture obtained from 0.0979 mmol of compound I3 and concentrated to dryness. CH₂Cl₂ and water were added and the aqueous layer was extracted with CH₂Cl₂. The combined organic layers were dried over Na₂SO₄ and concentrated to dryness. The residue was purified twice by flash chromatography over silica gel (CH₂Cl₂/MeOH from 100:0 to 95:5, then reversed phase, water/MeCN from 75:25 to 0:100) to afford intermediate I3 as a pale pink solid (0.383 g, 63%).

Preparation of Intermediate I4

A mixture of intermediate I3 (300 mg, 0.81 mmol), 3-(trifluoromethoxy)phenylboronic acid (CAS [179113-90-7], 0.21 g, 1.02 mmol) and potassium phosphate monohydrate (584 mg, 2.53 mmol) in a mixture of 1,4-dioxane (3.2 mL) and water (0.8 mL) was purged with argon. [1,1′-Bis(diphenylphosphino)ferrocene] dichloropalladium(II) (61.8 mg, 0.0845 mmol) was then added and the resulting mixture was purged again with argon and stirred at 100° C. for 17 h. Water (50 mL) was added and the resulting precipitate was collected by filtration on a glass-frit and washed with water (30 mL) to afford a black solid, 0.312 g. It was purified by flash chromatography over silica gel (from 0 to 5% of MeOH in DCM). The desired collected fractions were concentrated under reduced pressure and the resulting solid was triturated with MeOH (3×2 mL) and vacuum-dried at 60° C. for 48 h to afford intermediate I4 a purple solid, 0.215 g (59%).

Preparation of Compound 23

A mixture of intermediate I4 (0.164 g, 0.365 mmol) and sodium thiomethoxide (0.0895 g, 1.28 mmol) in DMF (1 mL) was stirred at 80° C. for 1.5 hours, then allowed to cool back to room temperature. The reaction mixture was then diluted with dichloromethane (40 ml) and washed with aq. sat NH₄Cl (25 mL) and brine (5×25 mL). The organic layer was dried over Na₂SO₄, filtered and concentrated to dryness under reduced pressure. It was purified by flash chromatography over silica gel (dichloromethane/MeOH from 100/0 to 95/5) affording 0.125 g. It was purified by reverse flash chromatography over silica gel (water/acetonitrile from 58/42 to 48/52 in 20 min, then 48/52 to 40/60 in 25 min) affording 0.078 g as an off-white solid. It was purified in several portions by preparative HPLC (waters xbridge column C18, 5 μm, 30×150 mm; eluent: water (0.2 wt % NH₄HCO₃)/acetonitrile (65/35) for 40 min). The resulting product was co-evaporated with EtOH (5 ml), triturated with Et₂O (2 ml) and vacuum-dried (50° C., 22 h) yielding compound 23 0.015 g (9.5%) as an off-white solid.

¹H NMR (400 MHz DMSO-d₆) δ ppm 11.60 (s, 1H), 9.15 (s, 1H), 8.57 (s, 1H), 8.12 (dd, J=8.1, 1.4 Hz, 1H), 7.97 (d, J=8.4 Hz, 1H), 7.88-7.79 (m, 3H), 7.78 (s, 1H), 7.69 (t, J=8.1 Hz, 1H), 7.62 (ddd, J=8.5, 7.0, 1.4 Hz, 1H), 7.49-7.41 (m, 1H), 7.29 (dd, J=8.1, 7.0 Hz, 1H), 2.34 (s, 3H).

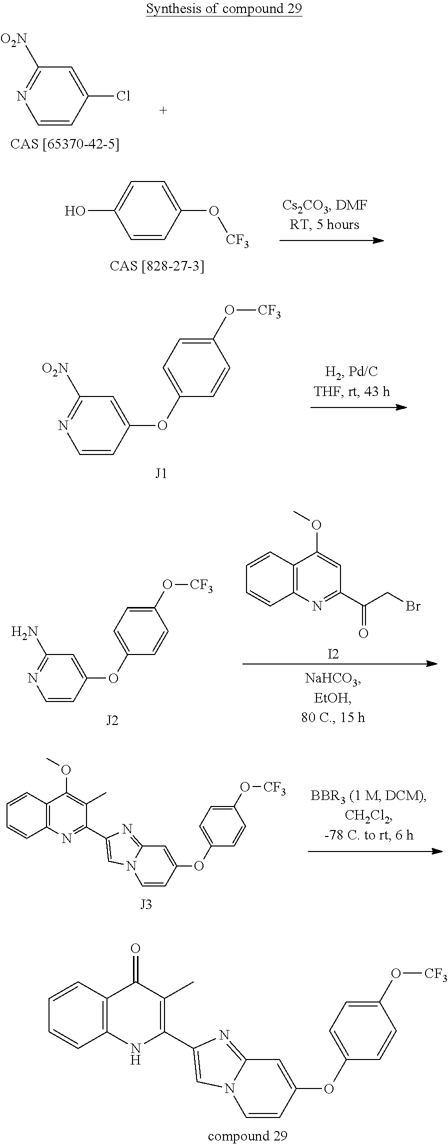

Preparation of Intermediate J1

To a solution of 4-chloro-2-nitropyridine (CAS [65370-42-5], 0.930 g, 5.87 mmol) in DMF (13 mL) were added 4-(trifluoromethoxy)phenol (CAS [828-27-3], 0.760 mL, 5.87 mmol) and Cs₂CO₃ (5.73 g, 17.6 mmol). The reaction mixture was stirred at room temperature for 5 h and then diluted with CH₂Cl₂ and water. The organic layer was washed with brine, dried over Na₂SO₄, filtered and concentrated to dryness. The crude residue was purified by flash chromatography over silica gel (cyclohexane/EtOAc from 100:0 to 50:50) to afford intermediate J1 as a yellow oil (0.344 g, 20%).

Preparation of Intermediate J2

A mixture of intermediate intermediate J1 (0.310 g, 1.03 mmol) in THF (2.7 mL) was purged with argon, then palladium on activated charcoal (10 wt. %, 0.110 g, 0.103 mmol) was added and the mixture was purged with argon and then with hydrogen and stirred under hydrogen atmosphere (1 atm) at room temperature for 23 h. Only partial conversion was observed, so the reaction mixture was filtered on a pad of Celite® which was rinsed with CH₂Cl₂. The filtrate was concentrated to dryness, THF (2.7 mL) was added and the mixture was purged with argon. Palladium on activated charcoal (10 wt. %, 0.110 g, 0.103 mmol) was then added and the mixture was purged with argon and then with hydrogen and stirred under hydrogen atmosphere (1 atm) at room temperature for 20 h. The reaction mixture was combined with another reaction mixture obtained from 0.100 mmol of intermediate L1 and filtered on a pad of Celite® which was rinsed with CH₂Cl₂. The filtrate was concentrated to dryness and the product was vacuum-dried to afford intermediate J2 as a brown solid (0.220 g, 72%).

Preparation of Intermediate J3

To a solution of crude compound I2 (0.226 g, max. 0.729 mmol) in EtOH (7.5 mL) were added intermediate J2 (0.197 g, 0.729 mmol) and NaHCO₃ (0.122 g, 1.46 mmol) and the mixture was stirred at 80° C. for 15 h. The reaction mixture was combined with another reaction mixture obtained from 0.0740 mmol of intermediate L2 and concentrated to dryness. CH₂Cl₂ and water were added and the aqueous layer was extracted with CH₂Cl₂. The combined organic layers were dried over Na₂SO₄, filtered and concentrated to dryness. The residue was purified by flash chromatography over silica gel (CH₂Cl₂/EtOAc from 100:0 to 50:50) to afford intermediate J3 as a pink wax (0.246 g, 66%).

Preparation of Compound 29

To a solution of intermediate J3 (0.222 g, 0.477 mmol) in CH₂Cl₂ (9.9 mL) was added boron tribromide (1 M in CH₂Cl₂) (2.39 ml, 2.39 mmol) dropwise at −78° C. under argon atmosphere and the mixture was warmed to room temperature and stirred for 6 h. The reaction mixture was quenched with water and diluted with CH₂Cl₂. The aqueous layer was extracted with CH₂Cl₂. The combined organic layers were washed with brine, dried over Na₂SO₄, filtered and concentrated to dryness. The crude residue was purified by flash chromatography over silica gel (IR50SI, CH₂Cl₂/EtOAc 100:0 to 0:100). The product was triturated in Et₂O and the resulting suspension was filtered. The solid was solubilized in MeOH and concentrated to dryness and then vacuum-dried at 50° C. to afford compound 29 as an orange solid (52.6 mg, 24%).

¹H NMR (400 MHz DMSO-d₆) δ ppm 11.45 (s, 1H), 8.71 (d, J=7.4 Hz, 1H), 8.53 (s, 1H), 8.11 (dd, J=8.2, 1.4 Hz, 1H), 7.92 (d, J=8.5 Hz, 1H), 7.60 (ddd, J=8.6, 6.8, 1.4 Hz, 1H), 7.50 (d, J=9.0 Hz, 2H), 7.34 (d, J=9.0 Hz, 2H), 7.27 (dd, J=8.1, 6.9 Hz, 1H), 7.03 (d, J=2.4 Hz, 1H), 6.96 (dd, J=7.4, 2.4 Hz, 1H), 2.31 (s, 3H).

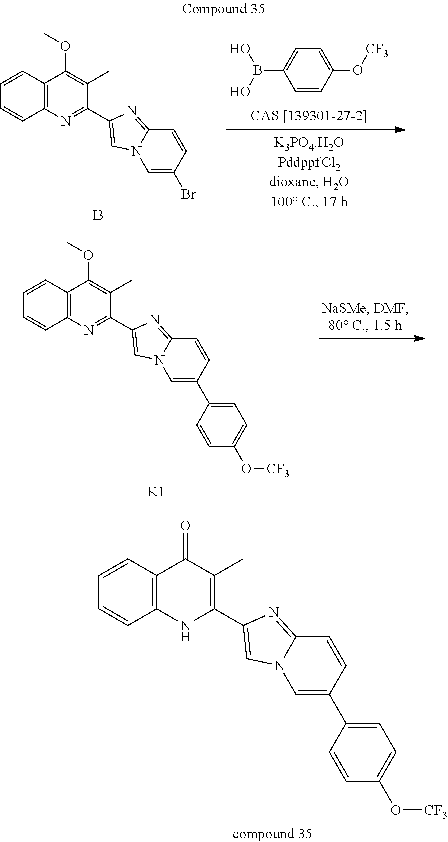

Preparation of Intermediate K1

Accordingly, intermediate K1 was prepared in the same way as intermediate intermediate I4 starting from intermediate I3 and 4-(trifluoromethoxy)phenylboronic acid (CAS [139301-27-2]). Intermediate K1 was obtained as a purple solid (0.145 g, 59%).

Preparation of Compound 35

A mixture of intermediate K1 (0.145 g, 0.323 mmol) and NaSMe (0.0791 g, 1.13 mmol) in DMF (1 mL) was stirred at 80° C. for 1 h then allowed to cool back to room temperature. The reaction mixture was then diluted with CH₂Cl₂ and washed with a saturated aqueous solution of NH₄Cl and brine. The organic layer was dried over Na₂SO₄, filtered and concentrated to dryness. The crude residue was purified by flash chromatography over silica gel (IR50SI, CH₂Cl₂/MeOH from 100:0 to 95:5), triturated with Et₂O and vacuum dried at 50° C. The product was purified by reversed phase flash chromatography (IR50C18, water/MeCN from 6:4 to 0:10) and then twice by preparative HPLC (waters xbridge column C18, 5 μm, 30×150 mm, MeCN/water 35:65+0.2 wt % NH₄HCO₃). The resulting residue was co-evaporated with EtOH, triturated with Et₂O and vacuum-dried at 50° C. to afford compound 35 as a brown solid (9.3 mg, 6.6%).

¹H NMR (400 MHz DMSO-d₆) δ ppm 11.59 (s, 1H), 9.09 (s, 1H), 8.58 (s, 1H), 8.12 (dd, J=8.1, 1.4 Hz, 1H), 7.97 (d, J=8.4 Hz, 1H), 7.89 (d, J=8.6 Hz, 2H), 7.83 (d, J=9.4 Hz, 1H), 7.78 (dd, J=9.4, 1.9 Hz, 1H), 7.62 (ddd, J=8.5, 6.9, 1.4 Hz, 1H), 7.55 (d, J=8.5 Hz, 2H), 7.29 (dd, J=8.1, 7.0 Hz, 1H), 2.34 (s, 3H).

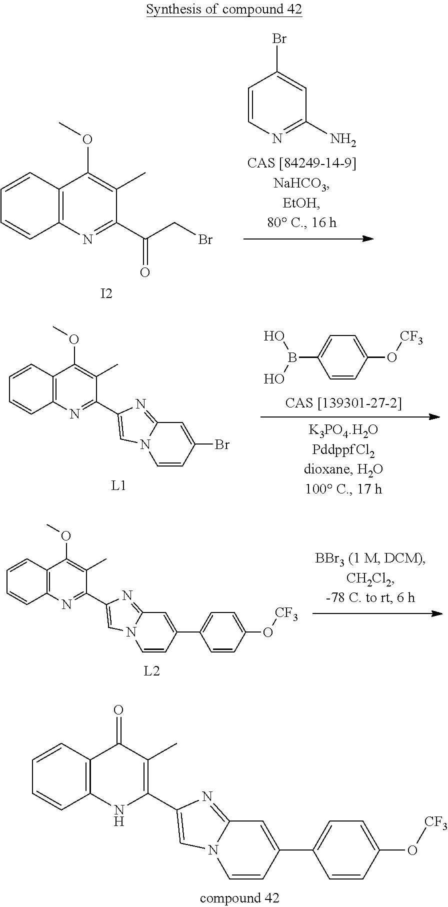

Preparation of Intermediate L1

Accordingly, intermediate L1 was prepared in the same way as intermediate I3 starting form intermediate I2 and 4-(trifluoromethoxy)phenylboronic acid (CAS [139301-27-2]). Intermediate L1 was obtained as a pale pink solid (0.383 g, 63%).

Preparation of Intermediate L2

Accordingly, intermediate L2 was prepared in the same way as intermediate I4 starting form intermediate L1 and 2-amino-4-bromopyridine (CAS [84249-14-9]). Intermediate L2 was obtained as a purple solid (0.191 g, quant).

Preparation of Compound 42

To a solution of intermediate L2 (0.165 g, 0.367 mmol) in CH₂Cl₂ (8 mL) was added BBr₃ (1 M in CH₂Cl₂, 1.84 mL, 1.84 mmol) dropwise at −78° C. under argon atmosphere and the mixture was warmed to room temperature and stirred for 3 h. The reaction mixture was quenched with water and combined with another reaction mixture obtained from 0.0445 mmol of intermediate N2. The mixture was diluted with CH₂Cl₂ and the aqueous layer was extracted with CH₂Cl₂. The combined organic layers were washed with brine, dried over Na₂SO₄ and concentrated to dryness. The crude residue was purified by reversed phase flash chromatography (water/MeCN from 60:40 to 0:100). The product was solubilized in MeOH and then Et₂O was added. The supernatant was removed and the residual solid was co-evaporated with MeOH (3 times) and vacuum dried at 50° C. The residue was co-evaporated with MeOH (2 times) and then with EtOH and vacuum dried at 50° C. The residue was co-evaporated again with EtOH (3 times) and vacuum dried at 50° C. to afford compound 42 as a white solid (98.4 mg, 55%).

¹H NMR (400 MHz DMSO-d₆) δ ppm 11.58 (s, 1H), 8.77 (d, J=7.2 Hz, 1H), 8.61 (s, 1H), 8.13 (dd, J=8.1, 1.3 Hz, 1H), 8.04-7.97 (m, 3H), 7.95 (d, J=8.4 Hz, 1H), 7.62 (ddd, J=8.4, 6.9, 1.5 Hz, 1H), 7.53 (d, J=8.7 Hz, 2H), 7.46 (dd, J=7.2, 1.9 Hz, 1H), 7.29 (dd, J=8.1, 7.0 Hz, 1H), 2.33 (s, 3H).

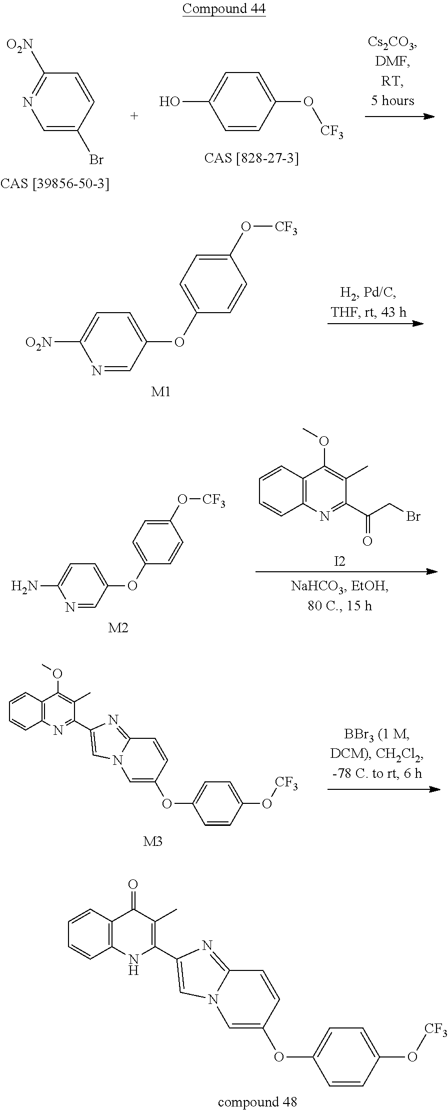

Preparation of Intermediate M1

Accordingly, intermediate M1 was prepared in the same way as intermediate J1. Starting from 5-bromo-2-nitropyridine (CAS [39856-50-3]) and 4-(trifluoromethoxy)phenol (CAS [828-27-3]). Intermediate M1 was obtained as yellow liquid (1.25 g, 92%).

Preparation of Intermediate M2

Accordingly, intermediate M2 was prepared in the same way as intermediate J2. Starting from intermediate M1. Intermediate M2 was obtained as a brown solid (1.05 g, 98%).

Preparation of Intermediate M3

Accordingly, intermediate M3 was prepared in the same way as intermediate J3. Starting from intermediate intermediate M2 and intermediate I2. Intermediate M3 was obtained as a brown solid (0.389 g, 56%).

Preparation of Compound 44

Accordingly, compound 44 was prepared in the same way as compound 29 starting from intermediate M3. Compound 44 was obtained as a pink solid (0.177 g, 52%).

¹H NMR (400 MHz DMSO-d₆) δ ppm 11.56 (s, 1H), 8.63 (d, J=2.4 Hz, 1H), 8.53 (s, 1H), 8.11 (dd, J=8.1, 1.5 Hz, 1H), 7.96 (d, J=8.4 Hz, 1H), 7.79 (d, J=9.5 Hz, 1H), 7.61 (ddd, J=8.4, 7.0, 1.5 Hz, 1H), 7.43 (d, J=8.9 Hz, 2H), 7.36 (dd, J=9.7, 2.3 Hz, 1H), 7.31-7.22 (m, 3H), 2.30 (s, 3H).

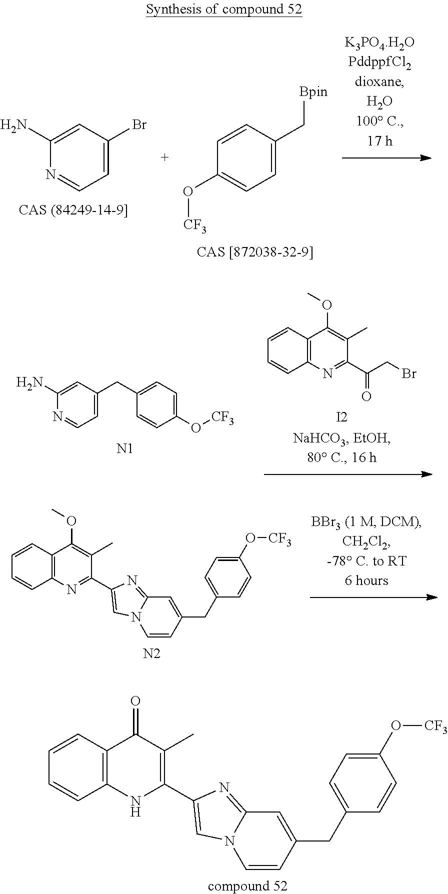

Preparation of Intermediate N1

A mixture of 2-amino-4-bromopyridine (CAS [84249-14-9], 0.400 g, 2.31 mmol), 4-(trifluoromethoxy)phenylmethylboronic acid, pinacol ester (CAS [872038-32-9], 0.838 g, 2.77 mmol) and K₃PO₄.H₂O (1.60 g, 6.94 mmol) in 1,4-dioxane (10.6 mL) and water (2.7 mL) was argon-purched, then Pd(dppf)Cl₂ (0.169 g, 0.231 mmol) was added and the mixture was purged again with argon and stirred at 100° C. for 2 h. The reaction mixture was filtered through a pad of Celite® which was rinsed with EtOAc and the filtrate was concentrated to dryness. The crude product intermediate N1 was considered as quantitative and used as such in the next step (1.09 g, containing maximum 2.31 mmol).

Preparation of Intermediate N2

To a solution of crude intermediate I2 (0.711 g, max. 2.30 mmol) in EtOH (24 mL) were added crude product intermediate N1 (1.08 g, max. 2.30 mmol) and NaHCO₃ (0.386 g, 4.59 mmol) and the mixture was stirred at 80° C. for 15 h. The reaction mixture was concentrated to dryness then CH₂Cl₂ and water were added and the aqueous layer was extracted with CH₂Cl₂. The combined organic layers were dried over Na₂SO₄, filtered and concentrated to dryness. The crude residue was purified by reversed phase flash chromatography (IR50C18, water/MeCN from 90:10 to 0:100) to afford intermediate N2 as a red wax (0.741 g, 63%).

Preparation of Compound 52

To a solution of intermediate intermediate N2 (0.707 g, 1.39 mmol) in CH₂Cl₂ (30.6 mL) was added BBr₃ (1 M in CH₂Cl₂) (6.94 mL, 6.94 mmol) dropwise at −78° C. under argon atmosphere and the mixture was warmed to room temperature and stirred for 23 h. The reaction mixture was quenched with water and diluted with CH₂Cl₂. The aqueous layer was extracted with CH₂Cl₂. The combined organic layers were washed with brine, dried over Na₂SO₄, filtered and concentrated to dryness. The crude residue was purified by flash chromatography over silica gel (IR50SI, CH₂Cl₂/EtOAc from 70:30 to 0:100 then CH₂Cl₂/MeOH from 100:0 to 90:10). The product was triturated in Et₂O, and the resulting suspension was filtered. The resulting solid was triturated with MeOH, concentrated to dryness (3 times) and then vacuum dried at 50° C. to afford compound 52 as an off-white solid (0.474 g, 76%).

¹H NMR (400 MHz DMSO-d₆) δ ppm 11.48 (s, 1H), 8.57 (d, J=7.0 Hz, 1H), 8.50 (s, 1H), 8.11 (dd, J=8.1, 1.5 Hz, 1H), 7.94 (d, J=8.4 Hz, 1H), 7.60 (ddd, J=8.4, 7.0, 1.5 Hz, 1H), 7.52 (s, 1H), 7.46 (d, J=8.5 Hz, 2H), 7.34 (d, J=8.5 Hz, 2H), 7.27 (dd, J=8.1, 7.0 Hz, 1H), 6.91 (dd, J=7.0, 1.7 Hz, 1H), 4.11 (s, 2H), 2.30 (s, 3H)

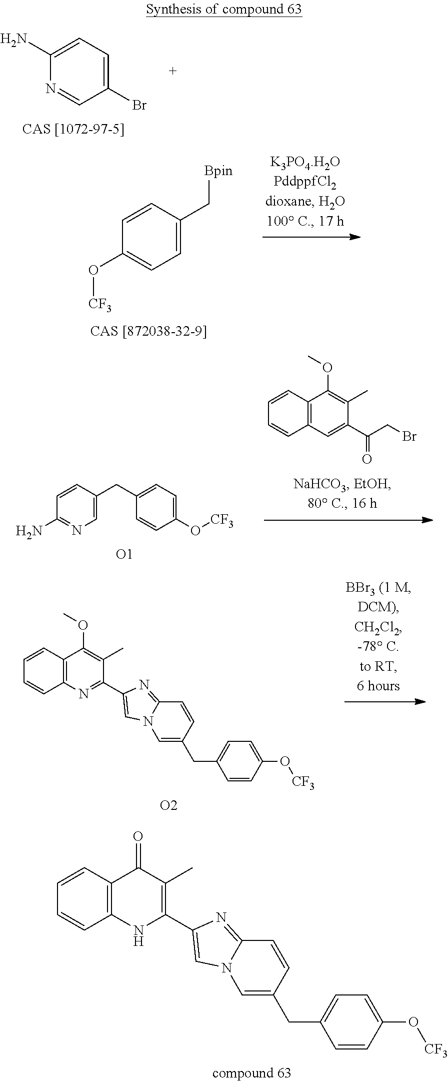

Preparation of Intermediate O1

Accordingly, intermediate O1 was prepared in the same way as intermediate N1. Starting from 2-amino-5-bromopyridine (CAS [1072-97-5]) and 4-(trifluoromethoxy)phenylmethylboronic acid, pinacol ester (CAS [872038-32-9]). Intermediate O1 was obtained as an orange solid (0.201 g, 65%).

Preparation of Intermediate O2

Accordingly, intermediate O2 was prepared in the same way as intermediate N2. Starting from intermediate O1 and intermediate I2. Intermediate O2 was obtained as a red sticky oil (0.297 g, 86%).

Preparation of Compound 63

Accordingly, compound 63 was prepared in the same way as compound 52 starting from intermediate O2. Compound 63 (was obtained as a brown solid (0.102 g, 35%).

¹H NMR (400 MHz DMSO-d₆) δ ppm 11.52 (s, 1H), 8.55 (s, 1H), 8.53 (s, 1H), 8.11 (dd, J=7.9, 1.5 Hz, 1H), 7.95 (d, J=8.4 Hz, 1H), 7.66 (d, J=9.4 Hz, 1H), 7.60 (ddd, J=8.4, 7.0, 1.5 Hz, 1H), 7.46 (d, J=8.5 Hz, 2H), 7.33 (d, J=8.5 Hz, 2H), 2.30 (s, 3H), 7.31-7.24 (m, 2H), 4.06 (s, 2H).

The following compounds depicted in the table below are/were also prepared in accordance with the methods described herein.

Analysis of Final Compounds

TABLE
LCMS methods used for final products (Flow expressed in mL/min; column temperature (T) in ° C.; Run time in minutes)

| | | melting | | | | | | |
|---|---|---|---|---|---|---|---|---|
| | | point | | | | | | |
| | | (DSC | | | | | | |
| | | Mettler | | | | | | |
| | cpd | Toledo | LC-MS | | | | | |
| | num- | (5° C./ | | UV | | BPM | BPM | Meth- |
| Structure | ber | min)) | RT | % | MW | 1 | 2 | od |
| 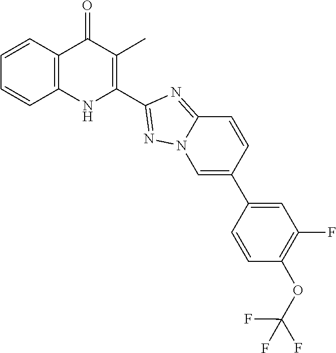 [SMILES: Cc1c(-c2nc3ccc(-c4ccc(OC(F)(F)F)c(F)c4)cn3n2)[nH]c2ccccc2c1=O] | 1 | 280.7° C. | 10.7 | 99.7 | 454.1 | 455 | | C |
| 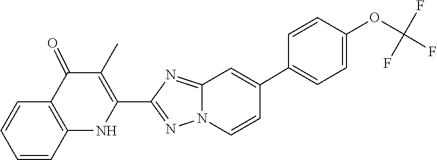 [SMILES: Cc1c(-c2nc3cc(-c4ccc(OC(F)(F)F)cc4)ccn3n2)[nH]c2ccccc2c1=O] | 2 | 274.8° C. | 8.5 | 97.1 | 354 | 355 | | C |
| 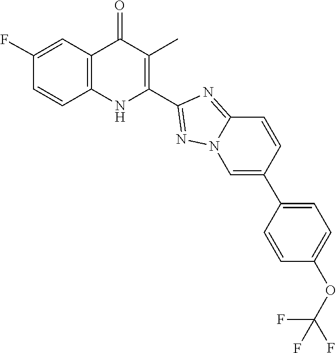 [SMILES: Cc1c(-c2nc3ccc(-c4ccc(OC(F)(F)F)cc4)cn3n2)[nH]c2ccc(F)cc2c1=O] | 3 | 257° C. | 8.6 | 99.2 | 384 | 385 | | C |
| 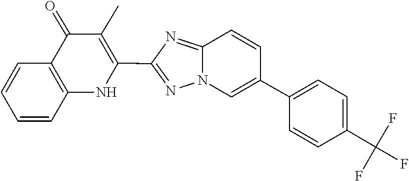 [SMILES: Cc1c(-c2nc3ccc(-c4ccc(C(F)(F)F)cc4)cn3n2)[nH]c2ccccc2c1=O] | 4 | 263.3° C. | 8.9 | 99.6 | 344.1 | 345 | | C |
| 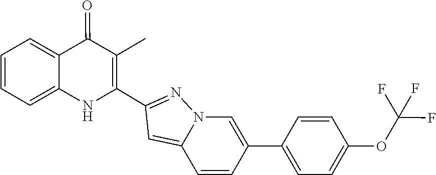 [SMILES: Cc1c(-c2cc3ccc(-c4ccc(OC(F)(F)F)cc4)cn3n2)[nH]c2ccccc2c1=O] | 5 | 322.0° C. | 7.7 | 99.1 | 301.1 | 302 | | C |
| 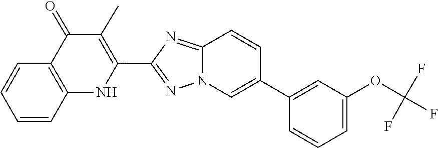 [SMILES: Cc1c(-c2nc3ccc(-c4cccc(OC(F)(F)F)c4)cn3n2)[nH]c2ccccc2c1=O] | 7 | 325° C. | 8.5 | 98.2 | 371.1 | 372 | | C |
| 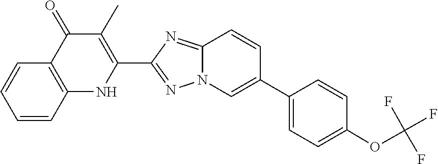 [SMILES: Cc1c(-c2nc3ccc(-c4ccc(OC(F)(F)F)cc4)cn3n2)[nH]c2ccccc2c1=O] | 6 | 261.9° C. | 10.6 | 100 | 435.1 | 436 | | C |
| 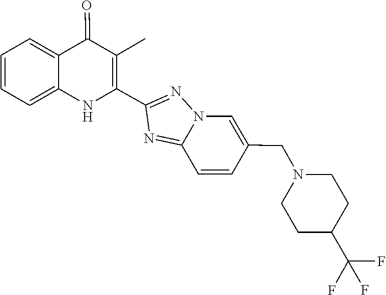 [SMILES: Cc1c(-c2nc3ccc(CN4CCC(C(F)(F)F)CC4)cn3n2)[nH]c2ccccc2c1=O] | 8 | 216.2° C. | 6.8 | 99.3 | 441.1 | 442.1 | | C |
| 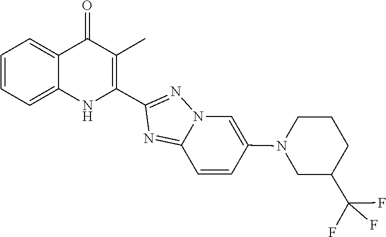 [SMILES: Cc1c(-c2nc3ccc(N4CCCC(C(F)(F)F)C4)cn3n2)[nH]c2ccccc2c1=O] | 9 | 270° C. | 10.2 | 99.2 | 427.1 | 428.1 | | C |
| 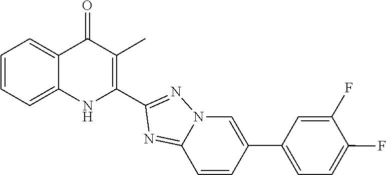 [SMILES: Cc1c(-c2nc3ccc(-c4ccc(F)c(F)c4)cn3n2)[nH]c2ccccc2c1=O] | 10 | 332.4° C. | 10 | 99.7 | 440.1 | 441 | | C |
| 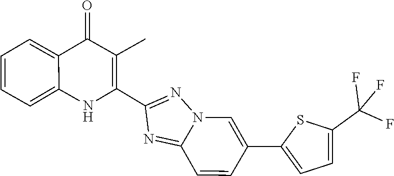 [SMILES: Cc1c(-c2nc3ccc(-c4ccc(C(F)(F)F)s4)cn3n2)[nH]c2ccccc2c1=O] | 11 | 299.3° C. | 9.8 | 98.9 | 427.1 | 428 | | C |
| 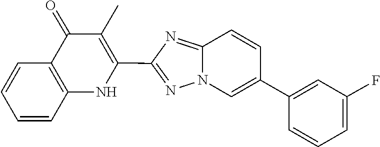 [SMILES: Cc1c(-c2nc3ccc(-c4cccc(F)c4)cn3n2)[nH]c2ccccc2c1=O] | 12 | 286.7° C. | 9.7 | 99 | 370.1 | 371 | | C |
| 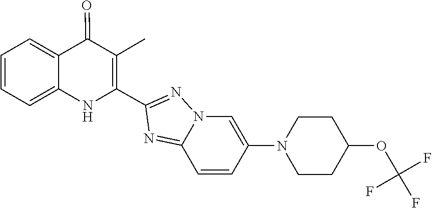 [SMILES: Cc1c(-c2nc3ccc(N4CCC(OC(F)(F)F)CC4)cn3n2)[nH]c2ccccc2c1=O] | 13 | see curve | 10.2 | 98.3 | 443.1 | 444.1 | | C |
| 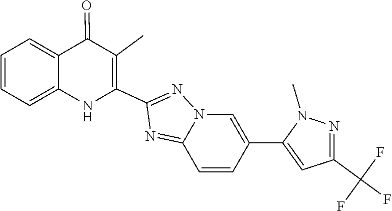 [SMILES: Cc1c(-c2nc3ccc(-c4cc(C(F)(F)F)nn4C)cn3n2)[nH]c2ccccc2c1=O] | 15 | 257.5° C. | 9.4 | 98.9 | 424.1 | 425.1 | | C |
| 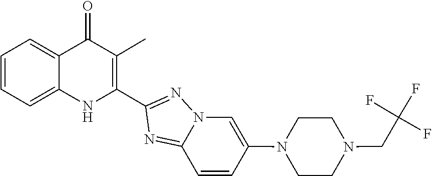 [SMILES: Cc1c(-c2nc3ccc(N4CCN(CC(F)(F)F)CC4)cn3n2)[nH]c2ccccc2c1=O] | 16 | 268.5° C. | 9.4 | 99.4 | 442.1 | 443.1 | | C |
| 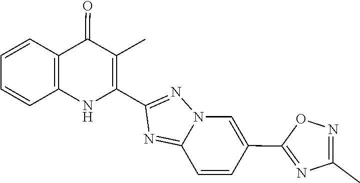 [SMILES: Cc1noc(-c2ccc3nc(-c4[nH]c5ccccc5c(=O)c4C)nn3c2)n1] | 17 | see curve | 8.3 | 98.9 | 358.1 | 359.1 | | C |
| 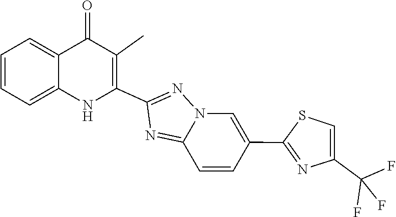 [SMILES: Cc1c(-c2nc3ccc(-c4nc(C(F)(F)F)cs4)cn3n2)[nH]c2ccccc2c1=O] | 18 | 323.5° C. | 9.9 | 96.6 | 388.1 | 389 | | C |
| 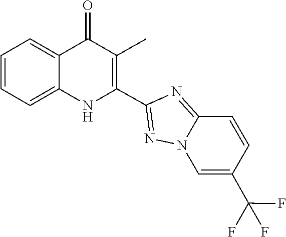 [SMILES: Cc1c(-c2nc3ccc(C(F)(F)F)cn3n2)[nH]c2ccccc2c1=O] | 19 | 268.9° C. | 7.7 | 99.2 | 294.1 | 295 | | C |
| 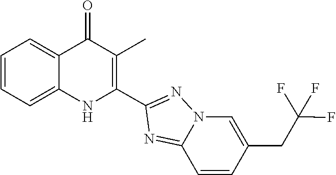 [SMILES: Cc1c(-c2nc3ccc(CC(F)(F)F)cn3n2)[nH]c2ccccc2c1=O] | 20 | 281.9° C. | 8.8 | 99.9 | 358.1 | 359 | | C |
| 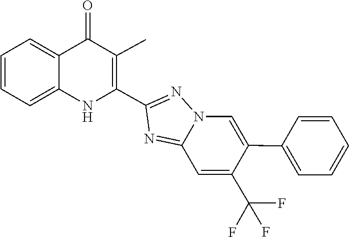 [SMILES: Cc1c(-c2nc3cc(C(F)(F)F)c(-c4ccccc4)cn3n2)[nH]c2ccccc2c1=O] | 21 | 283.0° C. | 10.3 | 100 | 496.1 | 497 | | C |
| 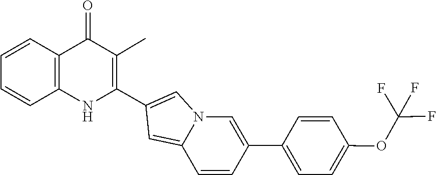 [SMILES: Cc1c(-c2cc3ccc(-c4ccc(OC(F)(F)F)cc4)cn3c2)[nH]c2ccccc2c1=O] | 22 | 243.7° C. | 10.7 | 98.8 | 435.1 | 436 | | C |
| 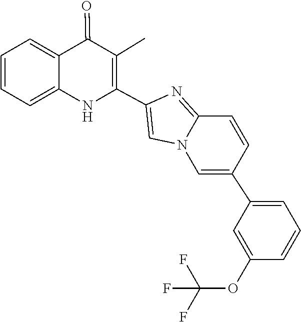 [SMILES: Cc1c(-c2cn3cc(-c4cccc(OC(F)(F)F)c4)ccc3n2)[nH]c2ccccc2c1=O] | 23 | see curve | 10.6 | 100 | 435.1 | 436 | | C |
| 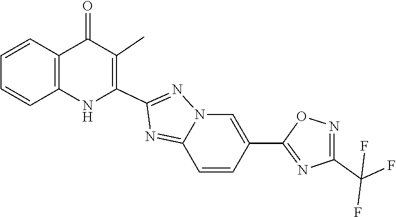 [SMILES: Cc1c(-c2nc3ccc(-c4nc(C(F)(F)F)no4)cn3n2)[nH]c2ccccc2c1=O] | 24 | 286.4° C. | 9.8 | 99.5 | 412 | 413 | | C |
| 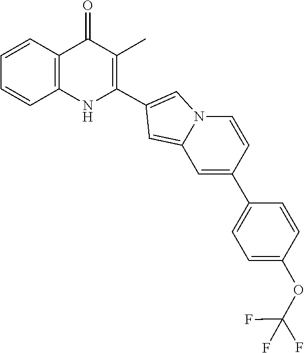 [SMILES: Cc1c(-c2cc3cc(-c4ccc(OC(F)(F)F)cc4)ccn3c2)[nH]c2ccccc2c1=O] | 25 | see curve | 6.8 | 97.6 | 434.1 | 435 | | D |
| 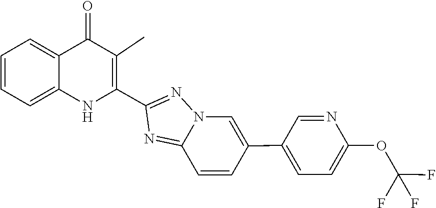 [SMILES: Cc1c(-c2nc3ccc(-c4ccc(OC(F)(F)F)nc4)cn3n2)[nH]c2ccccc2c1=O] | 26 | 314.9° C. | 9.9 | 99.7 | 436.1 | 438 | | C |
| 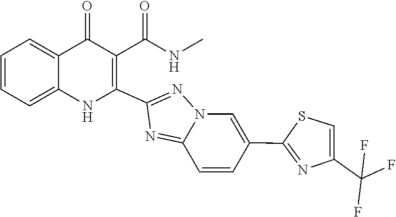 [SMILES: CNC(=O)c1c(-c2nc3ccc(-c4nc(C(F)(F)F)cs4)cn3n2)[nH]c2ccccc2c1=O] | 27 | 327.5° C. | 8.3 | 98.2 | 470 | 471 | | C |
| 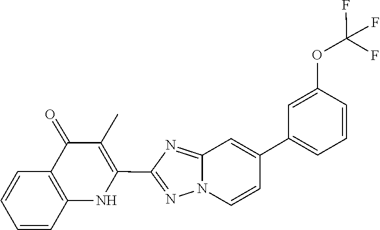 [SMILES: Cc1c(-c2nc3cc(-c4cccc(OC(F)(F)F)c4)ccn3n2)[nH]c2ccccc2c1=O] | 28 | 299.8° C. | 10.5 | 99.3 | 420.1 | 421 | | C |
| 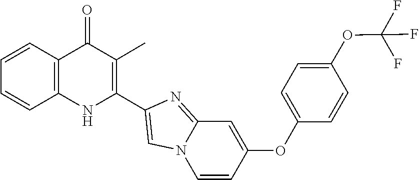 [SMILES: Cc1c(-c2cn3ccc(Oc4ccc(OC(F)(F)F)cc4)cc3n2)[nH]c2ccccc2c1=O] | 29 | 236.2° C. | 10.4 | 97.6 | 451.1 | 452 | | C |
| 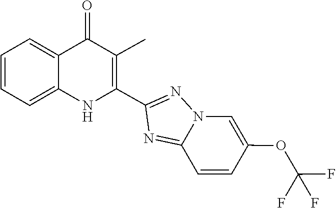 [SMILES: Cc1c(-c2nc3ccc(OC(F)(F)F)cn3n2)[nH]c2ccccc2c1=O] | 30 | 214.8° C. | 9 | 99.8 | 360 | 361 | | C |
| 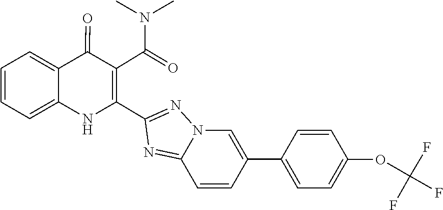 [SMILES: CN(C)C(=O)c1c(-c2nc3ccc(-c4ccc(OC(F)(F)F)cc4)cn3n2)[nH]c2ccccc2c1=O] | 31 | 332.8° C. | 9.5 | 97.5 | 493.1 | 494 | | C |
| 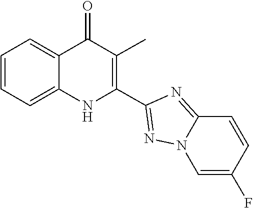 [SMILES: Cc1c(-c2nc3ccc(F)cn3n2)[nH]c2ccccc2c1=O] | 33 | 295.3° C. | 10.5 | 99.8 | 420.1 | 421 | | C |
| 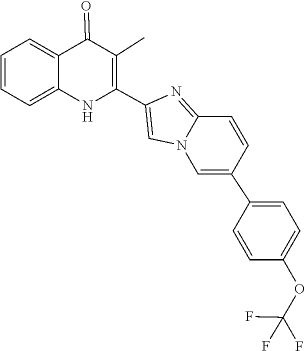 [SMILES: Cc1c(-c2cn3cc(-c4ccc(OC(F)(F)F)cc4)ccc3n2)[nH]c2ccccc2c1=O] | 35 | 235.9° C. | 10.6 | 99.6 | 435.1 | 436 | | C |
| 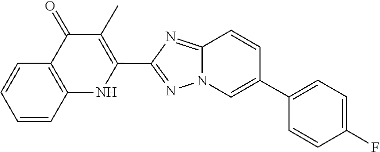 [SMILES: Cc1c(-c2nc3ccc(-c4ccc(F)cc4)cn3n2)[nH]c2ccccc2c1=O] | 36 | 325.6° C. | 9.7 | 97 | 370.1 | 371 | | C |
| 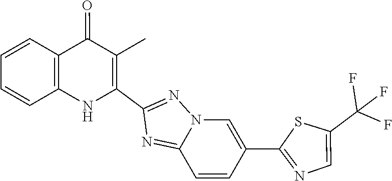 [SMILES: Cc1c(-c2nc3ccc(-c4ncc(C(F)(F)F)s4)cn3n2)[nH]c2ccccc2c1=O] | 37 | 331.3° C. | 9.4 | 97.3 | 428.1 | 429 | | C |
| 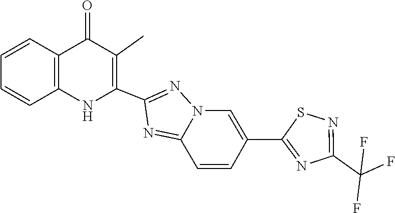 [SMILES: Cc1c(-c2nc3ccc(-c4nc(C(F)(F)F)ns4)cn3n2)[nH]c2ccccc2c1=O] | 38 | see curve | 9.8 | 98.6 | 428.1 | 429 | | C |
| 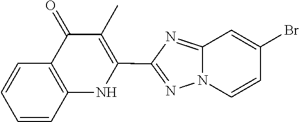 [SMILES: Cc1c(-c2nc3cc(Br)ccn3n2)[nH]c2ccccc2c1=O] | 39 | 342.0° C. | 9.7 | 97.6 | 422 | 423 | | C |
| 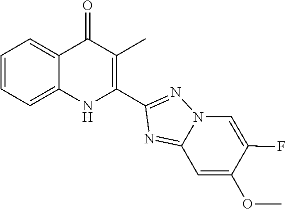 [SMILES: COc1cc2nc(-c3[nH]c4ccccc4c(=O)c3C)nn2cc1F] | 40 | 236.1° C. | 8.5 | 97.7 | 319.1 | 320 | | C |
| 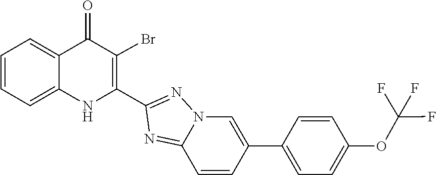 [SMILES: O=c1c(Br)c(-c2nc3ccc(-c4ccc(OC(F)(F)F)cc4)cn3n2)[nH]c2ccccc12] | 41 | 219.5° C. | 9.2 | 98.1 | 388.1 | 389 | | C |
| 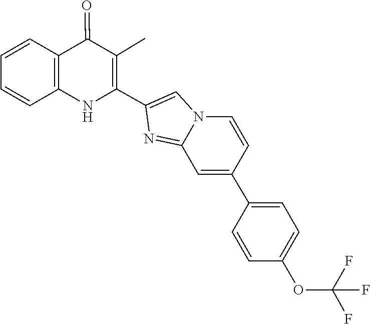 [SMILES: Cc1c(-c2cn3ccc(-c4ccc(OC(F)(F)F)cc4)cc3n2)[nH]c2ccccc2c1=O] | 42 | 263° C. | 10.6 | 99.1 | 435.1 | 436 | | C |
| 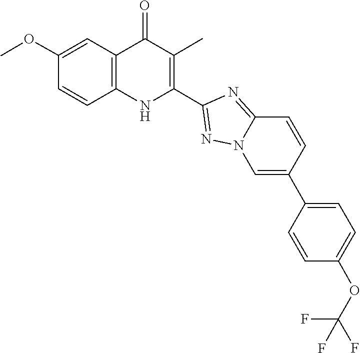 [SMILES: COc1ccc2[nH]c(-c3nc4ccc(-c5ccc(OC(F)(F)F)cc5)cn4n3)c(C)c(=O)c2c1] | 43 | 299.1° C. | 10.7 | 99.8 | 466.1 | 467 | | C |
| 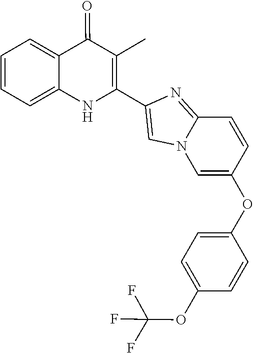 [SMILES: Cc1c(-c2cn3cc(Oc4ccc(OC(F)(F)F)cc4)ccc3n2)[nH]c2ccccc2c1=O] | 44 | see curve | 10.4 | 98.5 | 449.1 | 450 | | C |
| 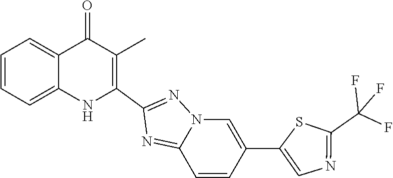 [SMILES: Cc1c(-c2nc3ccc(-c4cnc(C(F)(F)F)s4)cn3n2)[nH]c2ccccc2c1=O] | 45 | 221.3° C. | 7 | 99.5 | 347.1 | 348 | | C |
| 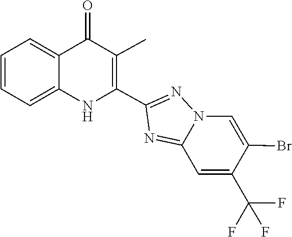 [SMILES: Cc1c(-c2nc3cc(C(F)(F)F)c(Br)cn3n2)[nH]c2ccccc2c1=O] | 46 | 273.5° C. | 8.2 | 97.5 | 414 | 415 | | C |
| 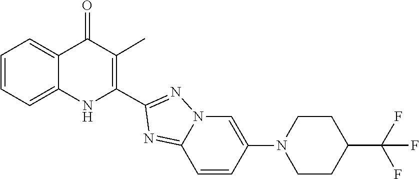 [SMILES: Cc1c(-c2nc3ccc(N4CCC(C(F)(F)F)CC4)cn3n2)[nH]c2ccccc2c1=O] | 47 | 284.5° C. | 9.6 | 100 | 352.1 | 353 | | C |
| 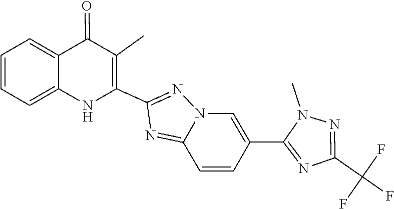 [SMILES: Cc1c(-c2nc3ccc(-c4nc(C(F)(F)F)nn4C)cn3n2)[nH]c2ccccc2c1=O] | 49 | 238.8° C. | 8.7 | 99.2 | 455.1 | 456 | | C |
| 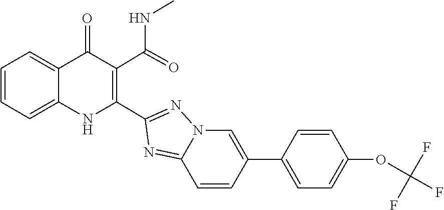 [SMILES: CNC(=O)c1c(-c2nc3ccc(-c4ccc(OC(F)(F)F)cc4)cn3n2)[nH]c2ccccc2c1=O] | 50 | see curve | 11 | 98.1 | 500.1 | 501 | | C |
| 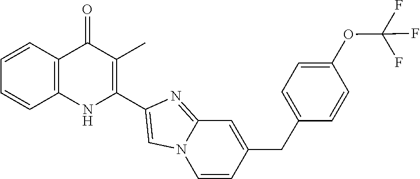 [SMILES: Cc1c(-c2cn3ccc(Cc4ccc(OC(F)(F)F)cc4)cc3n2)[nH]c2ccccc2c1=O] | 52 | 257.8° C. | 11.2 | 99.3 | 434.1 | 435 | | C |
| 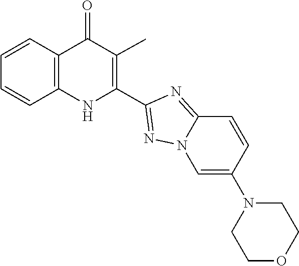 [SMILES: Cc1c(-c2nc3ccc(N4CCOCC4)cn3n2)[nH]c2ccccc2c1=O] | 54 | see curve | 7.7 | 98.4 | 361.1 | 362 | | C |
| 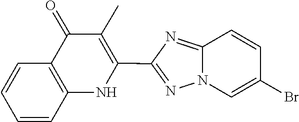 [SMILES: Cc1c(-c2nc3ccc(Br)cn3n2)[nH]c2ccccc2c1=O] | 55 | 233.1° C. | 8.5 | 100 | 354 | 335 | | C |
| 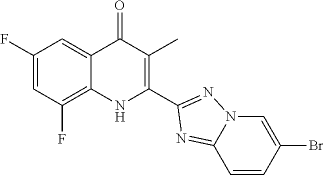 [SMILES: Cc1c(-c2nc3ccc(Br)cn3n2)[nH]c2c(F)cc(F)cc2c1=O] | 57 | 283.3° C. | 11.9 | 99.7 | 472 | 473 | | C |
| 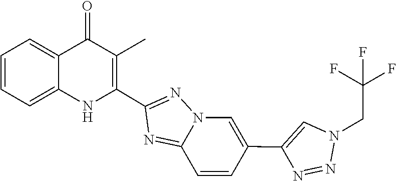 [SMILES: Cc1c(-c2nc3ccc(-c4cn(CC(F)(F)F)nn4)cn3n2)[nH]c2ccccc2c1=O] | 58 | 285.8° C. | 9 | 99.4 | 425.1 | 426 | | C |
| 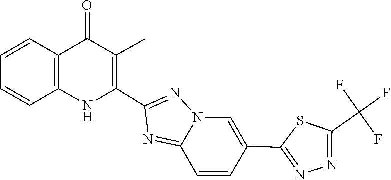 [SMILES: Cc1c(-c2nc3ccc(-c4nnc(C(F)(F)F)s4)cn3n2)[nH]c2ccccc2c1=O] | 59 | 282.8° C. | 10.6 | 100 | 426.1 | 427 | | C |
| 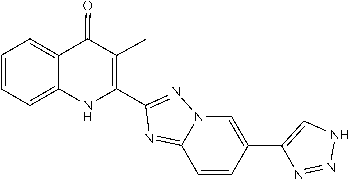 [SMILES: Cc1c(-c2nc3ccc(-c4c[nH]nn4)cn3n2)[nH]c2ccccc2c1=O] | 60 | see curve | 7.3 | 95.2 | 343.1 | 344.1 | | C |
| 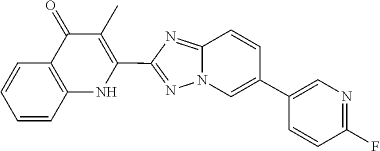 [SMILES: Cc1c(-c2nc3ccc(-c4ccc(F)nc4)cn3n2)[nH]c2ccccc2c1=O] | 61 | 223.7° C. | 10.6 | 100 | 436.1 | 437 | | C |
| 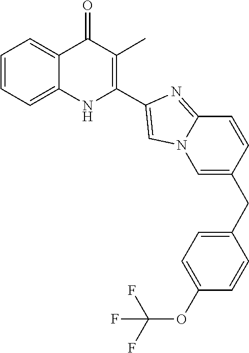 [SMILES: Cc1c(-c2cn3cc(Cc4ccc(OC(F)(F)F)cc4)ccc3n2)[nH]c2ccccc2c1=O] | 63 | 233.1° C. | 10.6 | 99.3 | 451.1 | 452 | | C |
| 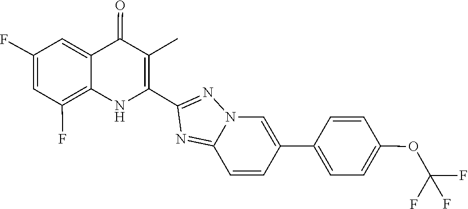 [SMILES: Cc1c(-c2nc3ccc(-c4ccc(OC(F)(F)F)cc4)cn3n2)[nH]c2c(F)cc(F)cc2c1=O] | 64 | 337.1° C. | 9.3 | 99.1 | 479.1 | 480 | | C |
| 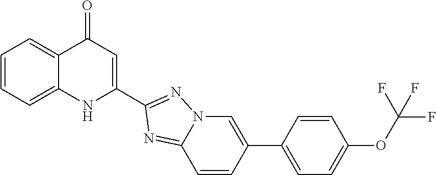 [SMILES: O=c1cc(-c2nc3ccc(-c4ccc(OC(F)(F)F)cc4)cn3n2)[nH]c2ccccc12] | 65 | see curve | 6.9 | 99.1 | 333.1 | 334 | | C |
| 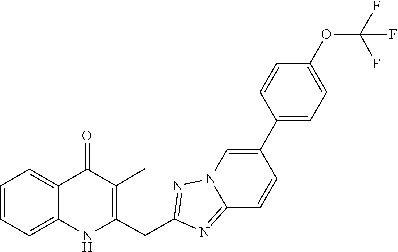 [SMILES: Cc1c(Cc2nc3ccc(-c4ccc(OC(F)(F)F)cc4)cn3n2)[nH]c2ccccc2c1=O] | 67 | see curve | 10.21 | 98.8 | 422.1 | 423 | | C |
| 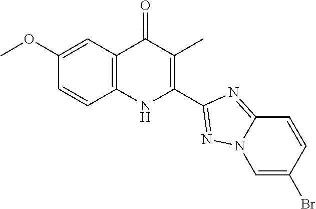 [SMILES: COc1ccc2[nH]c(-c3nc4ccc(Br)cn4n3)c(C)c(=O)c2c1] | 68 | 269.1° C. | 10.9 | 99.1 | 454.1 | 455 | | C |
| 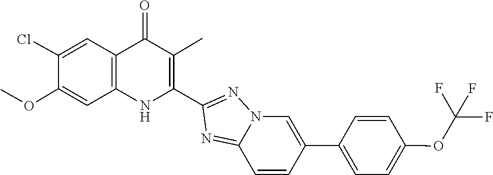 [SMILES: COc1cc2[nH]c(-c3nc4ccc(-c5ccc(OC(F)(F)F)cc5)cn4n3)c(C)c(=O)c2cc1Cl] | 69 | 278.4° C. | 11.4 | 98.1 | 500 | 501 | | C |
| 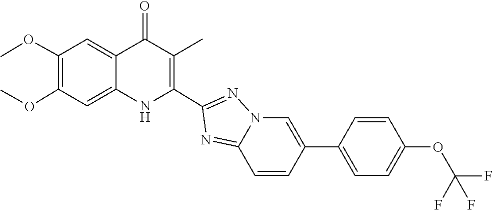 [SMILES: COc1cc2[nH]c(-c3nc4ccc(-c5ccc(OC(F)(F)F)cc5)cn4n3)c(C)c(=O)c2cc1OC] | 70 | 258.9° C. | 10.1 | 100 | 442.1 | 443 | | C |
| 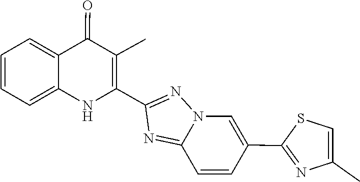 [SMILES: Cc1csc(-c2ccc3nc(-c4[nH]c5ccccc5c(=O)c4C)nn3c2)n1] | 71 | 344.6° C. | 7.4 | 99.2 | 357.1 | 358 | | C |
| 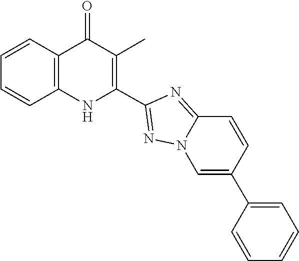 [SMILES: Cc1c(-c2nc3ccc(-c4ccccc4)cn3n2)[nH]c2ccccc2c1=O] | 72 | 257.4° C. | 11 | 99.6 | 435.1 | 436 | | C |
| 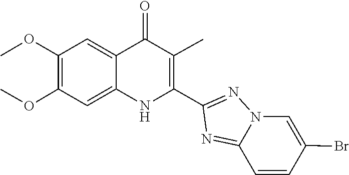 [SMILES: COc1cc2[nH]c(-c3nc4ccc(Br)cn4n3)c(C)c(=O)c2cc1OC] | 73 | 219.9° C. | 10.6 | 97.1 | 436.1 | 437 | | C |
| 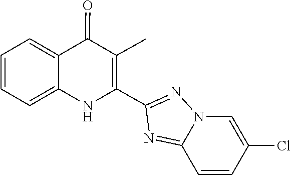 [SMILES: Cc1c(-c2nc3ccc(Cl)cn3n2)[nH]c2ccccc2c1=O] | 74 | 243.0° C. | 8.3 | 99.7 | 310 | 311 | | C |
| 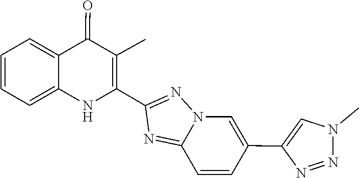 [SMILES: Cc1c(-c2nc3ccc(-c4cn(C)nn4)cn3n2)[nH]c2ccccc2c1=O] | 76 | 302.7° C. | 8.9 | 99.2 | 373.1 | 374 | | C |
| 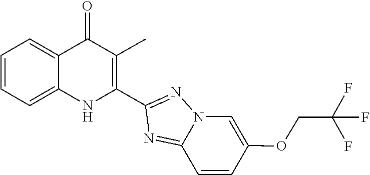 [SMILES: Cc1c(-c2nc3ccc(OCC(F)(F)F)cn3n2)[nH]c2ccccc2c1=O] | 77 | 276.9° C. | 10.61 | 99.1 | 500 | 501 | | C |
| 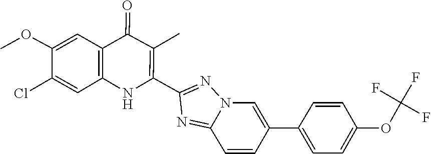 [SMILES: COc1cc2c(=O)c(C)c(-c3nc4ccc(-c5ccc(OC(F)(F)F)cc5)cn4n3)[nH]c2cc1Cl] | 78 | see curve | 11.5 | 96.68 | 500 | 501 | | C |
| 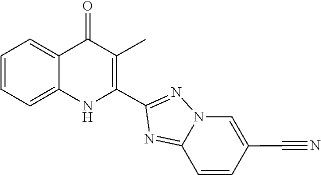 [SMILES: Cc1c(-c2nc3ccc(C#N)cn3n2)[nH]c2ccccc2c1=O] | 79 | 274.7° C. | 9.8 | 99.2 | 427 | 428 | | C |
| 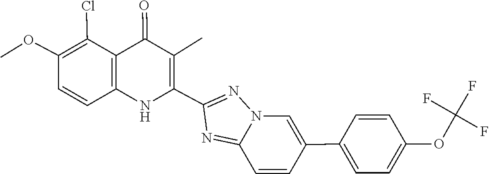 [SMILES: COc1ccc2[nH]c(-c3nc4ccc(-c5ccc(OC(F)(F)F)cc5)cn4n3)c(C)c(=O)c2c1Cl] | 80 | 287.1° C. | 9 | 99.7 | 374.1 | 375 | | C |
| 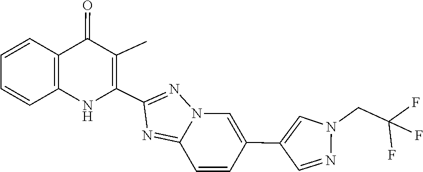 [SMILES: Cc1c(-c2nc3ccc(-c4cnn(CC(F)(F)F)c4)cn3n2)[nH]c2ccccc2c1=O] | 81 | 246.1° C. | 9.6 | 97.7 | 450.1 | 451 | | C |
| 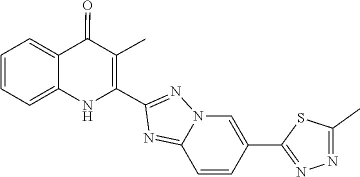 [SMILES: Cc1nnc(-c2ccc3nc(-c4[nH]c5ccccc5c(=O)c4C)nn3c2)s1] | 82 | 257.7° C. | 10 | 99.8 | 386.1 | 387 | | C |
| 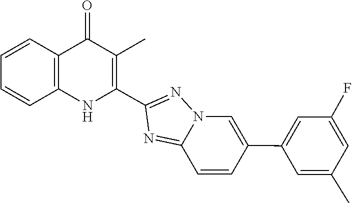 [SMILES: Cc1cc(F)cc(-c2ccc3nc(-c4[nH]c5ccccc5c(=O)c4C)nn3c2)c1] | 86 | 293.33° C. (DSC: 25° C. to 300° C./ 20° C. min/ 42 μl Al) | 2.92 | 100 | 384.1 | 385.2 | 383.1 | A |
| 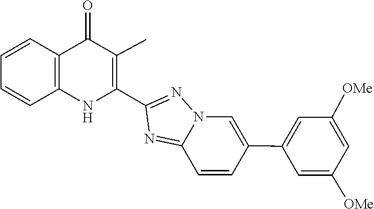 [SMILES: COc1cc(OC)cc(-c2ccc3nc(-c4[nH]c5ccccc5c(=O)c4C)nn3c2)c1] | 87 | 255.55° C. (DSC: 25° C. to 300° C./ 20° C. min/ 40 μl Al) | 2.79 | 100 | 412.1 | 413.2 | 411.1 | A |
| 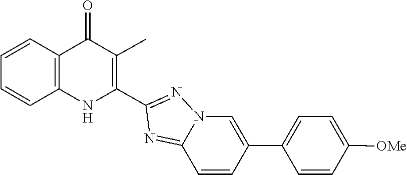 [SMILES: COc1ccc(-c2ccc3nc(-c4[nH]c5ccccc5c(=O)c4C)nn3c2)cc1] | 90 | 265.42° C. (DSC: 25° C. to 300° C./ 20° C. min/ 40 μl Al) | 2.75 | 100 | 382.1 | 383.2 | 381.1 | A |
| 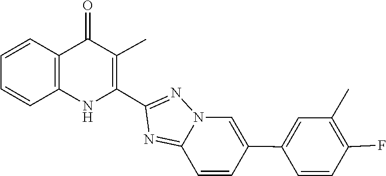 [SMILES: Cc1cc(-c2ccc3nc(-c4[nH]c5ccccc5c(=O)c4C)nn3c2)ccc1F] | 110 | 272.52° C. (DSC: 25° C. to 300° C./ 20° C. min/ 40 μl Al) | 2.97 | 99.1 | 384.1 | 385.2 | 383.1 | A |
| 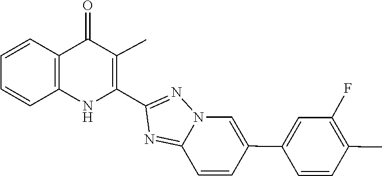 [SMILES: Cc1ccc(-c2ccc3nc(-c4[nH]c5ccccc5c(=O)c4C)nn3c2)cc1F] | 124 | 298.10° C. (DSC: 25° C. to 300° C./ 20° C. min/ 40 μl Al) | 1.41 | 95 | 384.1 | 385.2 | 383.3 | A |
| 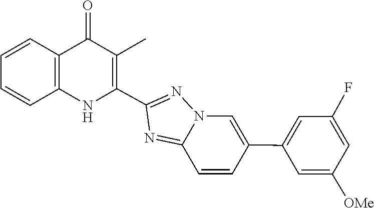 [SMILES: COc1cc(F)cc(-c2ccc3nc(-c4[nH]c5ccccc5c(=O)c4C)nn3c2)c1] | 125 | 250.19° C. (DSC: 25° C. to 300° C./ 20° C. min/ 40 μl Al) | 2.86 | 100 | 400.1 | 401.2 | 399.1 | A |
| 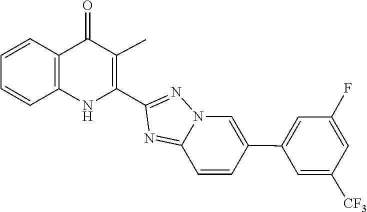 [SMILES: Cc1c(-c2nc3ccc(-c4cc(F)cc(C(F)(F)F)c4)cn3n2)[nH]c2ccccc2c1=O] | 126 | 296.18° C. (DSC: 25° C. to 300° C./ 20° C. min/ 40 μl Al) | 3.05 | 99.38 | 438.1 | 439.2 | 437.1 | A |
| 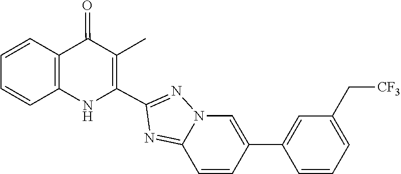 [SMILES: Cc1c(-c2nc3ccc(-c4cccc(CC(F)(F)F)c4)cn3n2)[nH]c2ccccc2c1=O] | 127 | 261.46° C. (DSC: 25° C. to 300° C./ 20° C. min/ 40 μl Al) | 2.94 | 99 | 434.1 | 435.3 | 433.2 | A |
| 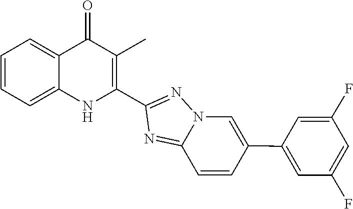 [SMILES: Cc1c(-c2nc3ccc(-c4cc(F)cc(F)c4)cn3n2)[nH]c2ccccc2c1=O] | 88 | | 2.59 | 100 | 388.1 | 389.1 | 387.3 | E |
| 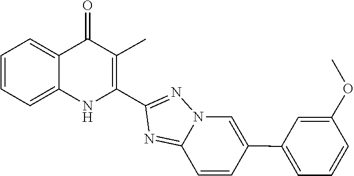 [SMILES: COc1cccc(-c2ccc3nc(-c4[nH]c5ccccc5c(=O)c4C)nn3c2)c1] | 89 | | 2.53 | 100 | 382.1 | 383.1 | 381.3 | E |
| 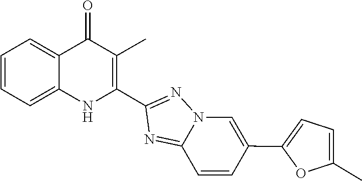 [SMILES: Cc1ccc(-c2ccc3nc(-c4[nH]c5ccccc5c(=O)c4C)nn3c2)o1] | 91 | | 2.54 | 77.63 | 356.1 | 357.1 | 355.2 | E |
| 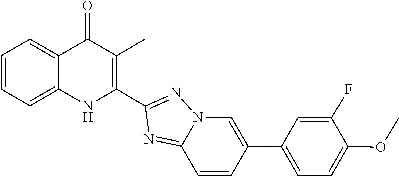 [SMILES: COc1ccc(-c2ccc3nc(-c4[nH]c5ccccc5c(=O)c4C)nn3c2)cc1F] | 98 | | 2.49 | 100 | 400.4 | 401.2 | 399.3 | E |
| 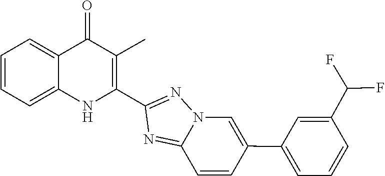 [SMILES: Cc1c(-c2nc3ccc(-c4cccc(C(F)F)c4)cn3n2)[nH]c2ccccc2c1=O] | 107 | | 2.53 | 100 | 402.4 | 403.2 | 401.3 | E |
| 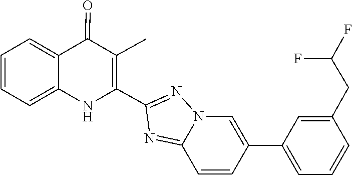 [SMILES: Cc1c(-c2nc3ccc(-c4cccc(CC(F)F)c4)cn3n2)[nH]c2ccccc2c1=O] | 112 | | 2.59 | 100 | 416.4 | 417.2 | 415.3 | E |
| 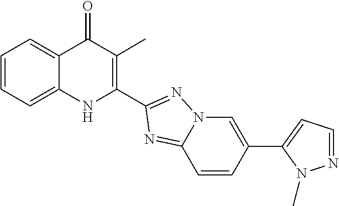 [SMILES: Cc1c(-c2nc3ccc(-c4ccnn4C)cn3n2)[nH]c2ccccc2c1=O] | 116 | | 1.88 | 100 | 356.4 | 357.2 | 355.2 | E |
| 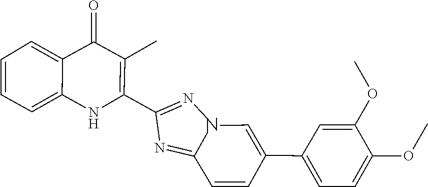 [SMILES: C/C=C(\C=C/C1=NC(c2[nH]c3ccccc3c(=O)c2C)=NC(CC)C1)c1ccc(OC)c(OC)c1] | 120 | | 2.34 | 100 | 412.4 | 413.2 | 411.3 | E |
| 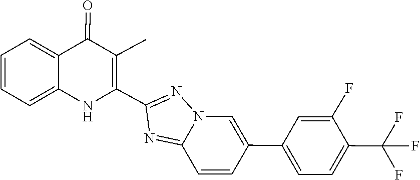 [SMILES: Cc1c(-c2nc3ccc(-c4ccc(C(F)(F)F)c(F)c4)cn3n2)[nH]c2ccccc2c1=O] | 123 | | 2.76 | 1000 | 438.4 | 439.1 | 437.3 | E |

TABLE
LCMS methods used for final products (Flow expressed in mL/min; column
temperature (T) in ° C.; Run time in minutes).

| | | | | | Flow | |
|---|---|---|---|---|---|---|
| | | | | | Column | Run |
| Method | Instrument | Column | Mobile phase | gradient | T | time |
| A | Waters: | Waters: BEH | A: 95% | 84.2% A to | 0.343 | 6.1 |
| | Acquity ® | C18 (1.7 μm, | CH3COONH4 | 10.5% A in | 40 | |
| | H-Class-DAD | 2.1 × 100 mm) | 7 mM/5% | 2.18 min, | | |
| | and SQD2TM | | CH3CN, | held for 1.96 | | |
| | | | B: CH3CN | min, back to | | |
| | | | | 84.2% A in | | |
| | | | | 0.73 min, | | |
| | | | | held for 0.73 | | |
| | | | | min. | | |
| B | Thermoscientific | Agilent: | A: HCOOH | 98% A for 3 | 1 | 28 |
| | Ultimate 3000 | Eclipse XDB | 0.1% in water/ | min, to 0% | 30 | |
| | DAD and | C18 | B: HCOOH | A in 12 min, | | |
| | Brucker HCT | (5 μm, | 0.05% in | held for 5 | | |
| | ultra | 4.6 × 150 mm) | CH3CN | min, back to | | |
| | | | | 98% A in 2 | | |
| | | | | min, held for | | |
| | | | | 6 min | | |
| C | Thermoscientific | Agilent: | A: HCOOH | 98% A for 2 | 1 | 18.4 |
| | Ultimate 3000 | Poroshell | 0.1% in water/ | min, to 0% | 30 | |
| | DAD and | EC-C18 | B: HCOOH | A in 10 min, | | |
| | Brucker HCT | (4 μm, | 0.05% in | held for 3.4 | | |
| | ultra | 4.6 × 100 mm) | CH3CN | min, back to | | |
| | | | | 98% A in | | |
| | | | | 1.3 min, | | |
| | | | | held for 1.7 | | |
| | | | | min | | |
| D | Thermoscientific | Agilent: | A: HCOOH | 50% A for 2 | 1 | 18.4 |
| | Ultimate 3000 | Poroshell | 0.1% in water/ | min, to 0% | 30 | |
| | DAD and | EC-C18 | B: HCOOH | A in 10 min, | | |
| | Brucker HCT | (4 μm, | 0.05% in | held for 3.4 | | |
| | ultra | 4.6 × 100 mm) | CH3CN | min, back to | | |
| | | | | 50% A in | | |
| | | | | 1.3 min, | | |
| | | | | held for | | |
| | | | | 1.7 min | | |
| E | Waters: Acquity | Waters: BEH | A: 95% | From 85% A | 0.35 | 6.1 |
| | UPLC ® | C18 (1.7 μm, | CH3COONH4 | to 10% A in | 40 | |
| | H-Class-DAD | 2.1 × 100 mm) | 7 mM/5% | 2.1 min, held | | |
| | and QDa | | CH3CN, | for 2 min, | | |
| | | | B: CH3CN | back to 85% | | |
| | | | | A in 0.8 min, | | |
| | | | | held for | | |
| | | | | 0.7 min. | | |

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl . . . ), the reported value is the one obtained for the lowest isotope mass. All results were obtained with experimental uncertainties that are commonly associated with the method used.

Hereinafter, “SQD” means Single Quadrupole Detector, “RT” room temperature, “BEH” bridged ethylsiloxane/silica hybrid, “HSS” High Strength Silica, “DAD” Diode Array Detector.

Reactions were in general carried out in anhydrous solvents under argon atmosphere if no other gas atmosphere was required.

NMR was carried out on a Bruker 400 MHz spectrometer or 500 MHz spectrometer.

Melting points were determined by DSC on a Mettler-Toledo DSC1 instrument (using aluminum standard 40 μL pans with air as purge gas and a thermal gradient between −10° C. and 350° C.) or on a melting point apparatus Buchi M-560, both applying indicated heating rates.

For flash chromatography, in general the following stationary phases were used: Interchim Silica gel IR-50SI (irregular, 50 μm), Interchim silica gel PF-15SIHP (spherical, 15 μm), Interchim C18-reversed silica gel IR-50C18 (irregular, 50 μm) or Buchi FlashPure silica gel (irregular, 50 μm).

### PHARMACOLOGICAL EXAMPLES

In the tests described below, individual compounds of the invention/examples (or combinations containing such compounds, for instance cytochrome bd inhibitors of the invention/examples in combination with one or more other inhibitor(s) of a (different) target of the electron transport chain of mycobacteria, as described herein) may be tested. For instance, in Tests 1 to 4, combinations may be tested (e.g. combinations of test cytochrome bd compounds with known cytochrome bc inhibitors, such as Q203 and Compound X). Where a control cytochrome bd compound is employed, then CK-2-63 is employed.

The compound Q203 (cytochrome bc1 inhibitor) may be prepared in accordance with the procedures in J. Medicinal Chemistry, 2014, 57 (12), pp 5293-5305, as well as, in WO 2011/113606 (see Compound 289 “6-chloro-2-ethyl-N-(4-(4-(4-(trifluoromethoxy)phenyl)piperidin-1-yl)benzyl)imidazo[1,2-a]pyridine-3-carboxamide”).

Compound X is 6-chloro-2-ethyl -N-({4-[2-(trifluoromethanesulfonyl)-2-azaspiro[3.3]heptan-6-yl]phenyl}methyl)imidazo[1,2-a]pyridine-3-carboxamide, which is described as Compound 154 of WO 2017/001660 and may be prepared according to the procedures described therein.

CK-2-63 may be prepared in accordance with the procedures disclosed in WO 2017/103615 (see experimental and the disclosures therein, referring to WO 2012/2069856, where an experimental procedure is provided for “3-methyl-2-(4-(4-(trifluoromethoxy)phenoxy)phenyl)quinolin-4(1H)-one”).

MIC Determination Against M. tuberculosis: Test 1

Test compounds and reference compounds were dissolved in DMSO and 1 μl of solution was spotted per well in 96 well plates at 200× the final concentration. Column 1 and column 12 were left compound-free, and from column 2 to 11 compound concentration was diluted 3-fold. Frozen stocks of Mycobacterium tuberculosis strain EH4.0 expressing green-fluorescent protein (GFP) were previously prepared and titrated. To prepare the inoculum, 1 vial of frozen bacterial stock was thawed to room temperature and diluted to 5×10 exp5 colony forming units per ml in 7H9 broth. 200 μl of inoculum, which corresponds to 1×10 exp5 colony forming units, were transferred per well to the whole plate, except column 12. 200 μl 7H9 broth were transferred to wells of column 12. Plates were incubated at 37° C. in plastic bags to prevent evaporation. After 7 days, fluorescence was measured on a Gemini EM Microplate Reader with 485 excitation and 538 nm emission wavelengths and IC₅₀ and/or pIC₅₀ values (or the like, e.g. IC₅₀, IC₉₀, pIC₉₀, etc) were (or may be) calculated.

MIC Determination Against M. tuberculosis: Test 2

Appropriate solutions of experimental and reference compounds were made in 96 well plates with 7H9 medium. Samples of Mycobacterium tuberculosis strain H37Rv were taken from cultures in logarithmic growth phase. These were first diluted to obtain an optical density of 0.3 at 600 nm wavelength and then diluted 1/100, resulting in an inoculum of approximately 5×10 exp5 colony forming units per ml. 100 μl of inoculum, which corresponds to 5×10 exp4 colony forming units, were transferred per well to the whole plate, except column 12. Plates were incubated at 37° C. in plastic bags to prevent evaporation. After 7 days, resazurin was added to all wells. Two days later, fluorescence was measured on a Gemini EM Microplate Reader with 543 excitation and 590 nm emission wavelengths and MIC₅₀ and/or pIC₅₀ values (or the like, e.g. IC₅₀, IC₉₀, pIC₉₀, etc) were (or may be) calculated.

Time Kill Kinetics Assays: Test 3

Bactericidal or bacteriostatic activity of the compounds can be determined in a time kill kinetic assay using the broth dilution method. In this assay, the starting inoculum of M. tuberculosis (strain H37Rv and H37Ra) is 10⁶ CFU/ml in Middlebrook (1×) 7H9 broth. The test compounds (cyt bd inhibitors) are tested in combination with a cyt bc inhibitor (for example Q203 or Compound X) at the concentration ranging from 10-30 μM to 0.9-0.3 μM respectively. Tubes receiving no antibacterial agent constitute the culture growth control. The tubes containing the microorganism and the test compounds are incubated at 37° C. After 0, 1, 4, 7, 14 and 21 days of incubation samples are removed for determination of viable counts by serial dilution (10⁰ to 10⁻⁶) in Middlebrook 7H9 medium and plating (100 μl) on Middlebrook 7H11 agar. The plates are incubated at 37° C. for 21 days and the number of colonies are determined. Killing curves can be constructed by plotting the log₁₀ CFU per ml versus time. A bactericidal effect of a cytochrome be and cytochrome bd inhibitor (either alone or in combination) is commonly defined as 2-log₁₀ decrease (decrease in CFU per ml) compared to Day 0. The potential carryover effect of the drugs is limited by using 0.4% charcoal in the agar plates, and by serial dilutions and counting the colonies at highest dilution possible used for plating.

Phenotypic Assay to Determine the O₂ Consumption Rate of Mycobacterium tuberculosis: Test 4

The aim of this assay is to evaluate the O₂ consumption rate of Mycobacterium tuberculosis (Mtb) bacilli after inhibition of cyt bc1 and cyt bd, using extracellular flux technology. Inhibition of cyt bc1 (e.g. using known inhibitors such as Q203 or Compound X) forces the bacillus to use the less energetically efficient terminal oxidase cyt bd. The inhibition of cyt bd will cause a significant decrease O₂ consumption. A sustained decrease of O₂ consumption under membrane potential disrupting conditions, via the addition of the uncoupler CCCP, will show to the efficacy of the cyt bd inhibitor. The oxygen consumption rate (OCR) of Mtb (stain H37Ra) bacilli adhered to the bottom of a Cell-Tak (BD Biosciences) coated XF cell culture microplate (Agilent), at 5×10⁶ bacilli per well, was measured using the Agilent Seahorse XFe96. Prior to the assay Mtb bacilli are cultured for two days to an OD₆₀₀ ˜0.7-0.9 in liquid medium, using 7H9 supplemented with 10% and 0.02% Tyloxapol. The assay media used is unbuffered 7H9 only supplemented with 0.2% glucose. For this assay the Compound X (final concentration of 0.9 μM, Compound X), is used to inhibit cyt bc1 and the cyt bd inhibitor, CK-2-63 (final concentration of 10 μM), is used as a positive control. The uncoupler CCCP is used at a final concentration of 1 μM.

In general, four basal OCR measurements are taken before the automatic addition of Compound X, through drug port A of the sensor cartridge, after which seven more OCR measurements are taken to allow enough time for the inhibition of cyt bc1. Next the cyt bd test compounds (final concentration of 10 μM), as well as the positive and negative controls (assay media with a final DMSO concentration of 0.4%), are added (drug port B) followed by seven OCR measurements. Finally, CCCP is added followed by three OCR measurements, this is done twice (drug ports C and D). For the control's measurements are performed in eight replicate wells and for the assay compounds six replicate wells per condition. Compounds are scored for their sustained inhibition of cyt bd in relation to the positive and negative controls.

Further Phenotypic Assay: Using a Cytochrome bc Knock-Out TB Strain and MIC Determination Against M. tuberculosis: Test 5

Appropriate solutions of experimental and reference compounds were made in 384 well plates with 7H9 medium. Samples of Mycobacterium tuberculosis strain H37Rv ΔctaE-ΔqcrCAB (Nat Commun 10, 4970, 2019, https://doi.org/10.1038/s41467-019-12956-2) were taken from cultures in logarithmic growth phase. These were first diluted to obtain an optical density of 0.4 at 600 nm wavelength and then diluted 1/150, resulting in an inoculum of approximately 5×10 exp5 colony forming units per ml. 30 μl of inoculum, which corresponds to 5×10 exp5 colony forming units, were transferred per well to the whole plate, except columns 23-24. Plates were incubated at 37° C., in an extra humidified incubator, in plastic bags to prevent evaporation. After 10 days, optical density at 620 nm wavelength was measured on an EnVision 2105 Multimode Plate Reader with a Photometric 620/8 excitation filter, and MIC₅₀ and/or pIC₅₀ values (or the like, e.g. IC₅₀, IC₉₀, pIC₉₀, etc) were (or may be) calculated.

Pharmacological Results

Biological Data—Example A

Compounds of the invention/examples (or combinations, e.g. compounds of the invention/examples in combination with one or more other inhibitors of a target of the electron transport chain), for example when tested in any of Tests 1 to 3, may display activity.

Biological Data—Example B

Compounds of the examples were tested in Test 4 described above (in section “Pharmacological Examples”; O₂ consumption rate testing), together with Compound X—a known cytochrome bc inhibitor—as described above, and the following results were obtained:

| | (i) % OCR after cyt | (ii) % OCR after |
|---|---|---|
| Example | bd inhibitor | cccp |
| 1 | 26.9 | 35.1 |
| 2 | 24.8 | 38.3 |
| 3 | 28.75 | 38.75 |
| 4 | 32.75 | 40.05 |
| 5 | 43.48 | 49.26 |
| 7 | 36.5 | 54.6 |
| 6 | 39.7 | 58.4 |
| 8 | 82.67 | 59.43 |
| 9 | 58.71 | 62.22 |
| 10 | 43.4 | 63.9 |
| 11 | 62.79 | 69.17 |
| 12 | 62.09 | 79.37 |
| 13 | 61.74 | 83.9 |
| 15 | 91.58 | 94.75 |
| 16 | 80.34 | 95.29 |
| 17 | 98.64 | 97.68 |
| 18 | 76.01 | 98.7 |
| 19 | 99.4 | 100.5 |
| 20 | 78.5 | 102.86 |
| 21 | 81.6 | 103.4 |
| 22 | 82.7 | 109.8 |
| 23 | 97.51 | 110.11 |
| 24 | 99.13 | 113.29 |
| 25 | 65.2 | 114.3 |
| 26 | 71 | 116.7 |
| 27 | 111.36 | 117.59 |
| 28 | 123.3 | 117.7 |
| 29 | 64.6 | 118.5 |
| 30 | 93.8 | 120.2 |
| 31 | 68.97 | 120.39 |
| 33 | 61.2 | 123.6 |
| 35 | 102.9 | 129.2 |
| 36 | 88.6 | 130.6 |
| 37 | 93.8 | 132.68 |
| 38 | 95.63 | 132.88 |
| 39 | 110.6 | 133.8 |
| 40 | 95.33 | 134.33 |
| 41 | 84.09 | 137.15 |
| 42 | 80.1 | 137.9 |
| 43 | 109.2 | 138 |
| 44 | 76 | 138.6 |
| 45 | 94.41 | 138.84 |
| 46 | 91.4 | 140.4 |
| 47 | 99.34 | 141.23 |
| 49 | 101.3 | 143.58 |
| 50 | 114.33 | 144.07 |
| 52 | 82.2 | 148.1 |
| 55 | 109.2 | 148.3 |
| 57 | 102 | 149.2 |
| 58 | 139.74 | 153.33 |
| 59 | 107.31 | 154.34 |
| 60 | 114.505 | 155.065 |
| 61 | 108 | 155.2 |
| 63 | 85.8 | 157.9 |
| 64 | 109.65 | 158.95 |
| 65 | 99.86 | 158.97 |
| 67 | 99.28 | 161.97 |
| 68 | 129.7 | 164.4 |
| 69 | 150.98 | 165.1 |
| 70 | 102.2 | 165.65 |
| 71 | 95.71 | 167.55 |
| 72 | 99.84 | 169.69 |
| 73 | 105.2 | 171.2 |
| 74 | 86.4 | 173.3 |
| 76 | 111.72 | 175.05 |
| 77 | 110.77 | 175.26 |
| 78 | 135.74 | 178.3 |
| 79 | 112 | 180.5 |
| 80 | 122.15 | 180.98 |
| 81 | 98.58 | 182.7 |
| 82 | 96.77 | 186.45 |
| 86 | 32,57 | 39,44 |
| 87 | 33,23 | 38,98 |
| 90 | 25,77 | 30,69 |
| 110 | 40,37 | 51,51 |
| 124 | 31,72 | 37,02 |
| 125 | 45,66 | 64,44 |
| 126 | 43,71 | 51,74 |
| 127 | 49,62 | 65,3 |
| 88 | 63,905 | 76 |
| 89 | 55,82 | 62,29 |
| 91 | 49,11 | 56,23 |
| 98 | 54,17 | 73,86 |
| 107 | 58,92 | 76,98 |
| 112 | 31,36 | 38,59 |
| 116 | 72,01 | 85,87 |
| 120 | 59,83 | 89,24 |
| 123 | 50,75 | 60,795 |

Biological Data—Example C

Compounds of the examples are/were tested in Test 3 (the kill kinetics) described above, obtaining results expressed as a log reduction in CFUs per ml as compared to Day 0. The following results were obtained.

| | | | Log Day 21- |
|---|---|---|---|
| | Log Day 0 | Log Day 21 | Log Day 0 |
| Control | 6.66 | 9.16 | +2.50 |
| Compound X (0.17 μg/ml) | 6.66 | 5.93 | −0.73 |
| Compound 6 (12 μg/ml) | 6.66 | 9.06 | +2.40 |
| Compound 7 (12 μg/ml) | 6.66 | 9.13 | +2.47 |
| Compound X (0.17 μg/ml) + | 6.66 | 1.40 | −5.26 |
| Compound 6 (12 μg/ml) | | | |
| Compound X (0.17 μg/ml) + | 6.66 | 2.27 | −4.39 |
| compound 6 (1.2 μg/ml) | | | |
| Compound X (0.17 μg/ml) + | 6.66 | 5.76 | −0.89 |
| compound 6 (0.12 μg/ml) | | | |
| Compound X (0.17 μg/ml) + | 6.66 | 1.40 | −5.26 |
| Compound 7 (12 μg/ml) | | | |
| Compound X (0.17 μg/ml) + | 6.66 | 1.30 | −5.36 |
| Compound 7 (1.2 μg/ml) | | | |
| Compound X (0.17 μg/ml) + | 6.66 | 5.39 | −1.27 |
| Compound 7 (0.12 μg/ml) | | | |

| | Log Day 0 | Log Day 21 | Log Day 21-Log Day 0 |
|---|---|---|---|
| Control | 5.56 | 8.73 | +3.17 |
| Q203 (0.168 μg/ml) | 5.56 | 2.59 | −2.97 |
| Compound 6 (12 μg/ml) | 5.56 | 8.64 | +3.08 |
| Compound 2 (12 μg/ml) | 5.56 | 8.69 | +3.13 |
| Q203 (0.168 μg/ml) + | 5.56 | 1.00 | −4.56 |
| compound 6 (12 μg/ml) | | | |
| Q203 (0.168 μg/ml) + | 5.56 | 1.00 | −4.56 |
| compound 2 (12 μg/ml) | | | |

Biological Data—Example D

Compounds of the examples were re-tested in Test 5 described above, and the following results were obtained:

| Compound | |
|---|---|
| number | pIC₅₀ |
| 35 | 5.622 |
| 23 | <4.000 |
| 42 | 4.513 |
| 44 | 5.011 |
| 63 | 4.118 |
| 52 | <4.000 |
| 29 | 4.017 |
| 22 | 5.426 |
| 25 | 5.510 |
| 6 | 6.185 |
| 2 | 5.876 |
| 55 | 4.443 |
| 29 | 5.023 |
| 4 | 5.904 |
| 33 | <4.000 |
| 39 | <4.000 |
| 46 | <4.301 |
| 73 | <4.000 |
| 30 | 5.535 |
| 28 | 4.998 |
| 1 | 5.787 |
| 21 | 4.945 |
| 70 | <4.301 |
| 57 | <4.301 |
| 12 | 6.053 |
| 68 | <4.000 |
| 43 | <4.000 |
| 74 | <4.000 |
| 7 | 5.851 |
| 26 | 5.114 |
| 18 | 5.726 |
| 79 | <4.000 |
| 5 | 5.896 |
| 54 | 5.226 |
| 72 | 6.584 |
| 20 | 5.663 |
| 40 | <4.000 |
| 65 | <5.000 |
| 41 | 4.470 |
| 77 | 5.734 |
| 80 | <4.602 |
| 67 | 4.989 |
| 81 | 4.778 |
| 45 | 5.225 |
| 11 | 5.585 |
| 38 | 5.312 |
| 76 | <5.301 |
| 71 | <5.000 |
| 82 | <5.301 |
| 31 | <4.301 |
| 49 | <4.301 |
| 58 | <4.602 |
| 17 | <5.301 |
| 15 | 4.244 |
| 27 | <4.301 |
| 9 | 5.884 |
| 8 | 5.432 |
| 24 | 5.530 |
| 16 | 5.932 |
| 60 | <4.301 |
| 13 | 5.385 |
| 86 | 5.881 |
| 87 | 6.261 |
| 88 | 5.272 |
| 89 | 6.241 |
| 90 | 5.828 |
| 91 | 5.760 |
| 98 | 5.704 |
| 107 | 5.479 |
| 110 | 5.423 |
| 112 | 5.732 |
| 116 | 5.338 |
| 120 | 6.157 |
| 123 | 5.383 |
| 124 | 5.771 |
| 125 | 6.178 |
| 126 | 4.965 |
| 127 | 5.491 |

Further Data

The compounds of the invention/examples may have advantages associated with in vitro potency, kill kinetics (i.e. bactericidal effect) in vitro, PK properties, food effect, safety/toxicity (including liver toxicity, coagulation, 5-LO oxygenase), metabolic stability, Ames II negativity, MNT negativity, aqueous based solubility (and ability to formulate) and/or cardiovascular effect e.g. on animals (e.g. anesthetized guinea pig). The data below that was generated/calculated may be obtained using standard methods/assays, for instance that are available in the literature or which may be performed by a supplier (e.g. Microsomal Stability Assay—Cyprotex, Mitochondrial toxicity (Glu/Gal) assay—Cyprotex, as well as literature CYP cocktail inhibition assays).

Mitotoxicity data:

| cpd | | | Δ IC50,glu/ | |
|---|---|---|---|---|
| number | IC50, glu | IC50, gal | IC50,gal | Score |
| 1 | [x]100 | [x]19.8 | [x]5.06 | positive |
| 2 | [x]100 | [x]100 | [x]0 | negative |
| 4 | [x]50 | [x]50 | [x]0 | negative |
| 5 | [x]106.39 | [x]45.87 | [x]2.32 | inconclusive |
| 7 | [x]20 | [x]20 | [x]0 | negative |
| 6 | 26.5 | 22.1 | 1.2 | negative |
| 6 (repeat) | 15.6 | 21.9 | 1.4 | negative |
| 8 | [x]163.33 | [x]180 | [x]0.91 | negative |
| 12 | [x]100 | [x]100 | [x]0 | negative |
| 13 | [x]200 | [x] 141.1 | [x]1.42 | negative |
| 15 | [x]44.26 | [x]200 | [x]0.22 | negative |
| 16 | [x]200 | [x]200 | [x]0 | negative |
| 18 | [x]23.35 | [x]15.75 | [x]1.48 | negative |
| 19 | [x]100 | [x]100 | [x]0 | negative |
| 20 | [x] 176.1 | [x]125.31 | [x]1.41 | negative |
| 21 | [x]50 | [x]50 | [x]0 | negative |
| 22 | 139.5 | 9.9 | >13.3 | positive |
| 23 | 200 | 200 | n.a. | negative |
| 24 | [x]20 | [x]20 | [x]0 | negative |
| 25 | 200 | 9.9 | >20.2 | positive |
| 26 | [x]13.31 | [x]9.59 | [x]1.39 | negative |
| 27 | [x]100 | [x]100 | [x]0 | negative |
| 28 | [x]100 | [x]58.7 | [x]1.7 | negative |
| 29 | 79.7 | 53.7 | 1.5 | negative |
| 30 | [x]100 | [x]100 | [x]0 | negative |
| 31 | [x]200 | [x]75.95 | [x]2.63 | inconclusive |
| 33 | [x]100 | [x]100 | [x]0 | negative |
| 39 | [x]100 | [x]100 | [x]0 | negative |
| 40 | [x]200 | [x]200 | [x]0 | negative |
| 41 | [x]53.91 | [x]54.72 | [x]0.99 | negative |
| 42 | 200 | 200 | n.a. | negative |
| 43 | [x]26.3 | [x]10.6 | [x]2.48 | inconclusive |
| 44 | 47.8 | 58.5 | 0.8 | negative |
| 45 | [x]20 | [x]20 | [x]0 | negative |
| 46 | [x]100 | [x]100 | [x]0 | negative |
| 50 | [x]100 | [x]86.53 | [x]1.16 | negative |
| 52 | 133.5 | 108.0 | 1.2 | negative |
| 55 | [x]200;[x]100 | [x]200;[x]100 | [x]0;[x]0 | negative |
| 57 | [x]100 | [x]100 | [x]0 | negative |
| 58 | [x]100 | [x]100 | [x]0 | negative |
| 60 | [x]100 | [x]100 | [x]0 | negative |
| 63 | 200 | 96.8 | >1.7 | inconclusive |
| | | | | (precipitation) |
| 65 | [x]20 | [x]20 | [x]0 | negative |
| 67 | [x]30.03 | [x]0.39 | [x]76.87 | positive |
| 68 | [x]100 | [x]100 | [x]0 | negative |
| 70 | [x]100 | [x]27.1 | [x]3.69 | inconclusive |
| 72 | [x]100 | [x]100 | [x]0 | negative |
| 73 | [x]100 | [x]100 | [x]0 | negative |
| 74 | [x]134.52 | [x]121.15 | [x]l.ll | negative |
| 77 | [x]200 | [x]200 | [x]0 | negative |
| 79 | [x]100 | [x]100 | [x]0 | negative |
| 80 | [x]50 | [x]50 | [x]0 | negative |
| 81 | [x]200 | [x]200 | [x]0 | negative |
| 82 | [x]50 | [x]50 | [x]0 | negative |
| 86 | [x]50 | [x]50 | [x]0 | negative |
| 87 | [x]6.2 | [x]5.16 | [x]1.21 | inconclusive |
| 90 | [x]200 | [x]200 | [x]0 | negative |
| 110 | [x]200 | [x]200 | [x]0 | negative |
| 124 | [x]200 | [x]200 | [x]0 | negative |
| 125 | [x]200 | [x]200 | [x]0 | negative |
| 126 | [x]25 | [x]15.5 | [x]1.61 | inconclusive |
| 127 | [x]18.1 | [x]15.85 | [x]1.14 | negative |

In the table above, “negative” means that in the test, it was found to have low mitotoxicity (and hence no mitotoxicity alerts), “positive” means that there were some mitotoxicity alerts and “inconclusive” means that no accurate conclusion could be drawn, e.g. due to issues with the compound being tested in the assay, e.g. solubility or precipitation issues (e.g. compound may not be soluble enough or may precipitate).

In view of the data above, compounds of the invention/examples may be found to be advantageous as no mitotoxicity alerts were observed (e.g. in the Glu/Gal assay).

The following data were also generated:

Compound 6:

CVS—rCaCh, rNaCh & hERG IC₅₀ (μm)=>10/>10/>10

AMES II b (+/−rat S9)=negative

GSH and CN adducts=negative

PK parameters in mice T_{1/2}(h), CI (mL/min/kg), Fab %=5.6/1.69/64

CTCM Ca²⁺ transient h-cardiomyocytes HTS (μm)=0.1 μm, 0.2 μm, 0.5 μm, 1 μm, 2.5 μm, 5 μm (all no)

| | Cardio tox |
|---|---|
| | rCaCH (plC50), rNaCH (IC50), |
| Compound | hERG(DOF) (IC50) |
| 44 | 3.8, 1.2, 1.5 |
| 63 | >3.0, 2.8, >10 |
| 52 | ≈1.9, 7.4, >10 |
| 29 | 3.3, 2.3, ≈9.1 |
| 22 | >10, 6.9, >10 |
| 25 | >10, >10, >10 |
| 55 | >10, >10, >10 |
| 19 | >10, >10, >10 |
| 4 | >10, 2.3, >10 |
| 33 | >10, >10, >10 |
| 70 | >10, >10, >10 |
| 12 | >10, >10, >3.02 |
| 26 | >10, >10, >10 |
| 18 | >10, 1.51, >10 |
| 5 | >10, >10, >10 |
| 58 | >10, >10, >10 |

Compounds of the invention/examples, may therefore have the advantage that:
No in vitro cardiotoxicity is observed (for example either due to the CVS results or due to the Glu/Gal assay results, for instance low mitotoxicity (<3 in the Glu/Gal assay indicates no mitotoxicity alerts); and/or
No reactive metabolite formation is observed (e.g. GSH);

for instance as compared to other compounds, for instance prior art compounds.

1. A compound of formula (I): 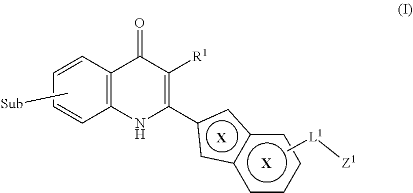
wherein:
R¹ is represents is C₁₋₆ alkyl, —Br, hydrogen or —C(O)N(R^{q1})R^{q2};
R^{q1} and R^{q2} are, independently, hydrogen or C₁₋₆ alkyl, or are linked together to form a 3-6 membered carbocyclic ring optionally substituted by one or more C₁₋₃ alkyl substituents;
Sub are one or more optional substituents that are halo, —CN, C₁₋₆ alkyl, or —O—C₁₋₆ alkyl (wherein the latter two alkyl moieties are optionally substituted by one or more fluoro atoms);
the two “X” rings together area 9-membered bicyclic heteroaryl ring that contains between one and four heteroatoms, and optionally substituted by one or more substituents that are halo and C₁₋₆ alkyl (itself optionally substituted by one or more fluoro atoms);
L¹ is an optional linker group;
R^{x1} and R^{x2} are, independently, hydrogen or C₁₋₃ alkyl;
Z¹ is one of (i) to (vi); 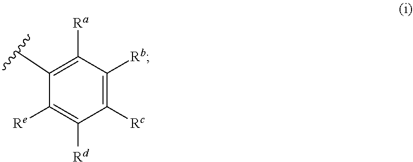 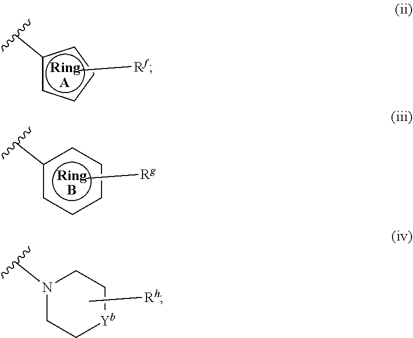
(v) perfluoro C₁₋₃ alkyl; or
(vi) —F, —Br, —Cl or —CN;
ring A is a 5-membered aromatic ring containing at least one heteroatom m), and is optionally substituted by one or more substituents that are, independently, R^{f};
ring B is a 6-membered aromatic ring containing at least one heteroatom and is optionally substituted by one or more substituents that are, independently, R^{g};
Y^{b} is —CH₂ or NH, and R^{h} is one or more substituents on the 6-membered N and Y^{b}-containing ring (which R^{h} substituents may also be present on Y^{b});
R^{a}, R^{b}, R^{c}, R^{d} and R^{e} are, independently, hydrogen or a substituent that is B¹;
each R^{f}, each R^{g} and each R^{h} (which are optional substituents), when present, are, independently, a substituent is B¹;
each B¹ is independently, a substituent that is:
(i) halo;
(ii) —R^{d1};
(iii) —OR^{e1};
(iv) —C(O)N(R^{e2})R^{e3}
(v) —SF₅;
(vi) —N(R^{e4})S(O)₂R^{e5};
R^{d1} is C₁₋₆ alkyl optionally substituted by one or more halo atoms;
R^{e1}, R^{e2}, R^{e3}, R^{e4} and R^{e5} are, independently, hydrogen or C₁₋₆ alkyl optionally substituted by one or more fluoro atoms,
or a pharmaceutically-acceptable salt thereof.

2. The compound of claim 1, wherein R¹ is C₁₋₃ alkyl.

3. The compound of in claim 1, wherein the “X” rings:
contain at least one nitrogen atom; and/or
contains one, two, three or four heteroatoms in total.

4. The compound of claim 1, wherein the “X” rings together are of formulae (IB): 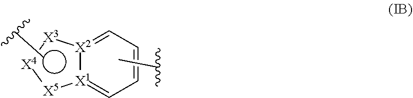
wherein:
one of X¹ and X² is N and the other is C;
X³, X⁴ and X⁵ are C, CH, or a heteroatom; and/or
none, one or two of X³, X⁴ and X⁵ are a heteroatom and the other is C (or CH) or CH.

5. The compound of claim 1, wherein:
L¹ is a direct bond, —O—, —C(R^{x1})(R^{x2})— or —OCH₂—;
R^{x1} and R^{x2} are, independently, hydrogen.

6. The compound of claim 1, wherein:
none, one or two of R^{a}, R^{b}, R^{c}, R^{d} and R^{e} is B¹ and the others are hydrogen; and/or
one of R^{b}, R^{c} and R^{d} is B¹ and the others are hydrogen.

7. The compound of claim 1, wherein B¹ a substituent that is:
(i) fluoro;
(ii) —OR^{e1};
(iii) C₁₋₃ alkyl substituted by one or more fluoro atom;
(iv) —C(O)N(R^{e2})R^{e3};
(v) —N(R^{e4})S(O)₂R^{e5}; or
(vi) —SF₅.

8. The compound of claim 1, wherein:
R^{e2} and R^{e4} independently are hydrogen; and
R^{e1}, R^{e3} and R^{e5} are, independently C₁₋₃ alkyl optionally substituted by one or more fluoro atoms.

9. (canceled)

10. A pharmaceutical composition comprising a pharmaceutically acceptable carrier and, as active ingredient, a therapeutically effective amount of the compound of claim 1.

11. (canceled)

12. (canceled)

13. A method of treatment of tuberculosis, comprising administration of a therapeutically effective amount of the compound of claim 1.

14. A combination of (a) the compound of claim 1, and (b) one or more other anti-tuberculosis agent.

15. A product containing (a) a compound of claim 1, and (b) one or more other anti-tuberculosis agent, as a combined preparation for simultaneous, separate or sequential treatment of a bacterial infection.

16. (canceled)

17. (canceled)

18. A method of treatment of tuberculosis, comprising administration of a therapeutically effective amount of a combination of claim 14.

19. A method of enhancing activity of another anti-tuberculosis agent, comprising administering the compound of claim 1 in combination with the another anti-tuberculosis agent.

20. A process for the preparation of a compound of formula (I) of claim 1, comprising:
(i) conversion of a compound of formula (II): 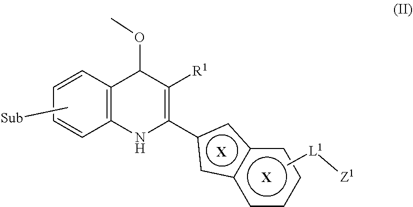
by reaction with BBr₃ or NaSCH₃; or
(ii) reaction of a compound of formula (III): 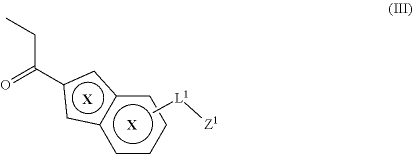
with a compound of formula (IV):

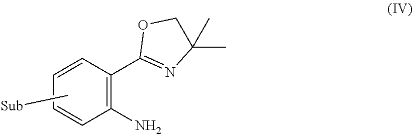

21. The compound of claim 1, wherein:
the 9-membered bicyclic heteroaryl ring consists of a 5-membered aromatic ring fused to another 6-membered aromatic ring; and/or
the 9-membered bicyclic heteroaryl ring contains between one and four nitrogen, oxygen, or sulfur; and/or
L¹ is a direct bond, —O—, —OCH₂—, —C(R^{x1})(R^{x2})— or —C(O)—N(H)—CH₂—;
Z¹ is CF₃;
Ring A contains at least one nitrogen atom;
Ring B contains at least one nitrogen atom;
R^{d1} is substituted by one or more fluoro atoms.

22. The compound of claim 2, wherein R¹ is methyl.

23. The compound of claim 3, wherein the “X” rings contain at least one nitrogen atom at the ring junction.

24. The compound of claim 4, wherein the heteroatom of X³, X⁴, and X⁵ is N, O, and/or S.

25. The compound of claim 6, wherein:
one or two of R^{a}, R^{b}, R^{c}, R^{d} and R^{e} is B¹ and the others are hydrogen; or
one of R^{a}, R^{b}, R^{c}, R^{d} and R^{e} is B¹ and the others are hydrogen; and/or
R^{c} is B¹ and R^{b} and R^{d} are hydrogen.

26. The compound of claim 8, wherein R^{e1}, R^{e3}, and R^{e5} are methyl.

27. The combination of claim 14, wherein the one or more other anti-tuberculosis agent is an inhibitor of the electron transport chain of mycobacteria.

28. The combination of claim 27, wherein the inhibitor of the electron transport chain of mycobacteria is a cytochrome bc inhibitor, an ATP synthase inhibitor, a NDH2 inhibitor and/or an inhibitor of the menaquinone synthesis pathway.

29. The combination of claim 28, wherein the inhibitor of the menaquinone synthesis pathway is a MenG inhibitor.

30. The product of claim 15, wherein the one or more other anti-tuberculosis agent is an inhibitor of the electron transport chain of mycobacteria.

31. The combination of claim 30, wherein the inhibitor of the electron transport chain of mycobacteria is a cytochrome bc inhibitor, an ATP synthase inhibitor, a NDH2 inhibitor and/or an inhibitor of the menaquinone synthesis pathway.

32. The combination of claim 31, wherein the inhibitor of the menaquinone synthesis pathway is a MenG inhibitor.

33. The compound of claim 1, wherein the “X” rings together form: [chemical structure]
